SEQUENCE LISTING (100 sequences)

SEQ ID NO: 1 - DNA, 85 bp, Artificial Sequence
  Synthetic
  modified_base (1)..(2): Biotin
  misc_feature (29)..(68): n is a, c, g, or t
aattttttttcccgtgttgctgtctgccnnnnnnnnnnnnnnnnnnnnnnnnnnnnnnnnnnnnnnnngcgggagcagggtagag

SEQ ID NO: 2 - DNA, 25 bp, Artificial Sequence
  Synthetic
atatatatcgccctcgtcccatctc

SEQ ID NO: 3 - DNA, 28 bp, Artificial Sequence
  Synthetic
  modified_base (1)..(2): Biotin
aattttttttcccgtgttgctgtctgcc

SEQ ID NO: 4 - DNA, 50 bp, Artificial Sequence
  Synthetic
  misc_feature (6)..(6): n is 5-(N-3-phenylpropylcarboxyamide) uracil
  misc_feature (8)..(8): n is 5-(N-3-phenylpropylcarboxyamide) uracil
  misc_feature (10)..(10): n is 5-(N-3-phenylpropylcarboxyamide) uracil
  misc_feature (13)..(13): n is 5-(N-3-phenylpropylcarboxyamide) uracil
  misc_feature (15)..(15): n is 5-(N-3-phenylpropylcarboxyamide) uracil
  misc_feature (17)..(20): n is 5-(N-3-phenylpropylcarboxyamide) uracil
  misc_feature (23)..(23): n is 5-(N-3-phenylpropylcarboxyamide) uracil
  misc_feature (25)..(25): n is 5-(N-3-phenylpropylcarboxyamide) uracil
  misc_feature (29)..(31): n is 5-(N-3-phenylpropylcarboxyamide) uracil
  misc_feature (34)..(34): n is 5-(N-3-phenylpropylcarboxyamide) uracil
  misc_feature (37)..(37): n is 5-(N-3-phenylpropylcarboxyamide) uracil
atctcngngncangngnnnnaangncagnnnaangangagggacgggcag

SEQ ID NO: 5 - DNA, 47 bp, Artificial Sequence
  Synthetic
  misc_feature (3)..(3): n is 5-(N-3-phenylpropylcarboxyamide) uracil
  misc_feature (5)..(5): n is 5-(N-3-phenylpropylcarboxyamide) uracil
  misc_feature (7)..(7): n is 5-(N-3-phenylpropylcarboxyamide) uracil
  misc_feature (10)..(10): n is 5-(N-3-phenylpropylcarboxyamide) uracil
  misc_feature (12)..(12): n is 5-(N-3-phenylpropylcarboxyamide) uracil
  misc_feature (14)..(17): n is 5-(N-3-phenylpropylcarboxyamide) uracil
  misc_feature (20)..(20): n is 5-(N-3-phenylpropylcarboxyamide) uracil
  misc_feature (22)..(22): n is 5-(N-3-phenylpropylcarboxyamide) uracil
  misc_feature (26)..(28): n is 5-(N-3-phenylpropylcarboxyamide) uracil
  misc_feature (31)..(31): n is 5-(N-3-phenylpropylcarboxyamide) uracil
  misc_feature (34)..(34): n is 5-(N-3-phenylpropylcarboxyamide) uracil
tcngngncangngnnnnaangncagnnnaangangagggacgggcag

SEQ ID NO: 6 - DNA, 44 bp, Artificial Sequence
  Synthetic
  misc_feature (2)..(2): n is 5-(N-3-phenylpropylcarboxyamide) uracil
  misc_feature (4)..(4): n is 5-(N-3-phenylpropylcarboxyamide) uracil
  misc_feature (7)..(7): n is 5-(N-3-phenylpropylcarboxyamide) uracil
  misc_feature (9)..(9): n is 5-(N-3-phenylpropylcarboxyamide) uracil
  misc_feature (11)..(14): n is 5-(N-3-phenylpropylcarboxyamide) uracil
  misc_feature (17)..(17): n is 5-(N-3-phenylpropylcarboxyamide) uracil
  misc_feature (19)..(19): n is 5-(N-3-phenylpropylcarboxyamide) uracil
  misc_feature (23)..(25): n is 5-(N-3-phenylpropylcarboxyamide) uracil
  misc_feature (28)..(28): n is 5-(N-3-phenylpropylcarboxyamide) uracil
  misc_feature (31)..(31): n is 5-(N-3-phenylpropylcarboxyamide) uracil
gngncangngnnnnaangncagnnnaangangagggacgggcag

SEQ ID NO: 7 - DNA, 47 bp, Artificial Sequence
  Synthetic
  misc_feature (6)..(6): n is 5-(N-3-phenylpropylcarboxyamide) uracil
  misc_feature (8)..(8): n is 5-(N-3-phenylpropylcarboxyamide) uracil
  misc_feature (10)..(10): n is 5-(N-3-phenylpropylcarboxyamide) uracil
  misc_feature (13)..(13): n is 5-(N-3-phenylpropylcarboxyamide) uracil
  misc_feature (15)..(15): n is 5-(N-3-phenylpropylcarboxyamide) uracil
  misc_feature (17)..(20): n is 5-(N-3-phenylpropylcarboxyamide) uracil
  misc_feature (23)..(23): n is 5-(N-3-phenylpropylcarboxyamide) uracil
  misc_feature (25)..(25): n is 5-(N-3-phenylpropylcarboxyamide) uracil
  misc_feature (29)..(31): n is 5-(N-3-phenylpropylcarboxyamide) uracil
  misc_feature (34)..(34): n is 5-(N-3-phenylpropylcarboxyamide) uracil
  misc_feature (37)..(37): n is 5-(N-3-phenylpropylcarboxyamide) uracil
atctcngngncangngnnnnaangncagnnnaangangagggacggg

SEQ ID NO: 8 - DNA, 44 bp, Artificial Sequence
  Synthetic
  misc_feature (6)..(6): n is 5-(N-3-phenylpropylcarboxyamide) uracil
  misc_feature (8)..(8): n is 5-(N-3-phenylpropylcarboxyamide) uracil
  misc_feature (10)..(10): n is 5-(N-3-phenylpropylcarboxyamide) uracil
  misc_feature (13)..(13): n is 5-(N-3-phenylpropylcarboxyamide) uracil
  misc_feature (15)..(15): n is 5-(N-3-phenylpropylcarboxyamide) uracil
  misc_feature (17)..(20): n is 5-(N-3-phenylpropylcarboxyamide) uracil
  misc_feature (23)..(23): n is 5-(N-3-phenylpropylcarboxyamide) uracil
  misc_feature (25)..(25): n is 5-(N-3-phenylpropylcarboxyamide) uracil
  misc_feature (29)..(31): n is 5-(N-3-phenylpropylcarboxyamide) uracil
  misc_feature (34)..(34): n is 5-(N-3-phenylpropylcarboxyamide) uracil
  misc_feature (37)..(37): n is 5-(N-3-phenylpropylcarboxyamide) uracil
atctcngngncangngnnnnaangncagnnnaangangagggac

SEQ ID NO: 9 - DNA, 41 bp, Artificial Sequence
  Synthetic
  misc_feature (6)..(6): n is 5-(N-3-phenylpropylcarboxyamide) uracil
  misc_feature (8)..(8): n is 5-(N-3-phenylpropylcarboxyamide) uracil
  misc_feature (10)..(10): n is 5-(N-3-phenylpropylcarboxyamide) uracil
  misc_feature (13)..(13): n is 5-(N-3-phenylpropylcarboxyamide) uracil
  misc_feature (15)..(15): n is 5-(N-3-phenylpropylcarboxyamide) uracil
  misc_feature (17)..(20): n is 5-(N-3-phenylpropylcarboxyamide) uracil
  misc_feature (23)..(23): n is 5-(N-3-phenylpropylcarboxyamide) uracil
  misc_feature (25)..(25): n is 5-(N-3-phenylpropylcarboxyamide) uracil
  misc_feature (29)..(31): n is 5-(N-3-phenylpropylcarboxyamide) uracil
  misc_feature (34)..(34): n is 5-(N-3-phenylpropylcarboxyamide) uracil
  misc_feature (37)..(37): n is 5-(N-3-phenylpropylcarboxyamide) uracil
atctcngngncangngnnnnaangncagnnnaangangagg

SEQ ID NO: 10 - DNA, 38 bp, Artificial Sequence
  Synthetic
  misc_feature (6)..(6): n is 5-(N-3-phenylpropylcarboxyamide) uracil
  misc_feature (8)..(8): n is 5-(N-3-phenylpropylcarboxyamide) uracil
  misc_feature (10)..(10): n is 5-(N-3-phenylpropylcarboxyamide) uracil
  misc_feature (13)..(13): n is 5-(N-3-phenylpropylcarboxyamide) uracil
  misc_feature (15)..(15): n is 5-(N-3-phenylpropylcarboxyamide) uracil
  misc_feature (17)..(20): n is 5-(N-3-phenylpropylcarboxyamide) uracil
  misc_feature (23)..(23): n is 5-(N-3-phenylpropylcarboxyamide) uracil
  misc_feature (25)..(25): n is 5-(N-3-phenylpropylcarboxyamide) uracil
  misc_feature (29)..(31): n is 5-(N-3-phenylpropylcarboxyamide) uracil
  misc_feature (34)..(34): n is 5-(N-3-phenylpropylcarboxyamide) uracil
  misc_feature (37)..(37): n is 5-(N-3-phenylpropylcarboxyamide) uracil
atctcngngncangngnnnnaangncagnnnaangang

SEQ ID NO: 11 - DNA, 35 bp, Artificial Sequence
  Synthetic
  misc_feature (6)..(6): n is 5-(N-3-phenylpropylcarboxyamide) uracil
  misc_feature (8)..(8): n is 5-(N-3-phenylpropylcarboxyamide) uracil
  misc_feature (10)..(10): n is 5-(N-3-phenylpropylcarboxyamide) uracil
  misc_feature (13)..(13): n is 5-(N-3-phenylpropylcarboxyamide) uracil
  misc_feature (15)..(15): n is 5-(N-3-phenylpropylcarboxyamide) uracil
  misc_feature (17)..(20): n is 5-(N-3-phenylpropylcarboxyamide) uracil
  misc_feature (23)..(23): n is 5-(N-3-phenylpropylcarboxyamide) uracil
  misc_feature (25)..(25): n is 5-(N-3-phenylpropylcarboxyamide) uracil
  misc_feature (29)..(31): n is 5-(N-3-phenylpropylcarboxyamide) uracil
  misc_feature (34)..(34): n is 5-(N-3-phenylpropylcarboxyamide) uracil
atctcngngncangngnnnnaangncagnnnaang

SEQ ID NO: 12 - DNA, 38 bp, Artificial Sequence
  Synthetic
  misc_feature (3)..(3): n is 5-(N-3-phenylpropylcarboxyamide) uracil
  misc_feature (5)..(5): n is 5-(N-3-phenylpropylcarboxyamide) uracil
  misc_feature (7)..(7): n is 5-(N-3-phenylpropylcarboxyamide) uracil
  misc_feature (10)..(10): n is 5-(N-3-phenylpropylcarboxyamide) uracil
  misc_feature (12)..(12): n is 5-(N-3-phenylpropylcarboxyamide) uracil
  misc_feature (14)..(17): n is 5-(N-3-phenylpropylcarboxyamide) uracil
  misc_feature (20)..(20): n is 5-(N-3-phenylpropylcarboxyamide) uracil
  misc_feature (22)..(22): n is 5-(N-3-phenylpropylcarboxyamide) uracil
  misc_feature (26)..(28): n is 5-(N-3-phenylpropylcarboxyamide) uracil
  misc_feature (31)..(31): n is 5-(N-3-phenylpropylcarboxyamide) uracil
  misc_feature (34)..(34): n is 5-(N-3-phenylpropylcarboxyamide) uracil
tcngngncangngnnnnaangncagnnnaangangagg

SEQ ID NO: 13 - DNA, 37 bp, Artificial Sequence
  Synthetic
  misc_feature (2)..(2): n is 5-(N-3-phenylpropylcarboxyamide) uracil
  misc_feature (4)..(4): n is 5-(N-3-phenylpropylcarboxyamide) uracil
  misc_feature (6)..(6): n is 5-(N-3-phenylpropylcarboxyamide) uracil
  misc_feature (9)..(9): n is 5-(N-3-phenylpropylcarboxyamide) uracil
  misc_feature (11)..(11): n is 5-(N-3-phenylpropylcarboxyamide) uracil
  misc_feature (13)..(16): n is 5-(N-3-phenylpropylcarboxyamide) uracil
  misc_feature (19)..(19): n is 5-(N-3-phenylpropylcarboxyamide) uracil
  misc_feature (21)..(21): n is 5-(N-3-phenylpropylcarboxyamide) uracil
  misc_feature (25)..(27): n is 5-(N-3-phenylpropylcarboxyamide) uracil
  misc_feature (30)..(30): n is 5-(N-3-phenylpropylcarboxyamide) uracil
  misc_feature (33)..(33): n is 5-(N-3-phenylpropylcarboxyamide) uracil
cngngncangngnnnnaangncagnnnaangangagg

SEQ ID NO: 14 - DNA, 36 bp, Artificial Sequence
  Synthetic
  misc_feature (1)..(1): n is 5-(N-3-phenylpropylcarboxyamide) uracil
  misc_feature (3)..(3): n is 5-(N-3-phenylpropylcarboxyamide) uracil
  misc_feature (5)..(5): n is 5-(N-3-phenylpropylcarboxyamide) uracil
  misc_feature (8)..(8): n is 5-(N-3-phenylpropylcarboxyamide) uracil
  misc_feature (10)..(10): n is 5-(N-3-phenylpropylcarboxyamide) uracil
  misc_feature (12)..(15): n is 5-(N-3-phenylpropylcarboxyamide) uracil
  misc_feature (18)..(18): n is 5-(N-3-phenylpropylcarboxyamide) uracil
  misc_feature (20)..(20): n is 5-(N-3-phenylpropylcarboxyamide) uracil
  misc_feature (24)..(26): n is 5-(N-3-phenylpropylcarboxyamide) uracil
  misc_feature (29)..(29): n is 5-(N-3-phenylpropylcarboxyamide) uracil
  misc_feature (32)..(32): n is 5-(N-3-phenylpropylcarboxyamide) uracil
ngngncangngnnnnaangncagnnnaangangagg

SEQ ID NO: 15 - DNA, 37 bp, Artificial Sequence
  Synthetic
  misc_feature (3)..(3): n is 5-(N-3-phenylpropylcarboxyamide) uracil
  misc_feature (5)..(5): n is 5-(N-3-phenylpropylcarboxyamide) uracil
  misc_feature (7)..(7): n is 5-(N-3-phenylpropylcarboxyamide) uracil
  misc_feature (10)..(10): n is 5-(N-3-phenylpropylcarboxyamide) uracil
  misc_feature (12)..(12): n is 5-(N-3-phenylpropylcarboxyamide) uracil
  misc_feature (14)..(17): n is 5-(N-3-phenylpropylcarboxyamide) uracil
  misc_feature (20)..(20): n is 5-(N-3-phenylpropylcarboxyamide) uracil
  misc_feature (22)..(22): n is 5-(N-3-phenylpropylcarboxyamide) uracil
  misc_feature (26)..(28): n is 5-(N-3-phenylpropylcarboxyamide) uracil
  misc_feature (31)..(31): n is 5-(N-3-phenylpropylcarboxyamide) uracil
  misc_feature (34)..(34): n is 5-(N-3-phenylpropylcarboxyamide) uracil
tcngngncangngnnnnaangncagnnnaangangag

SEQ ID NO: 16 - DNA, 36 bp, Artificial Sequence
  Synthetic
  misc_feature (3)..(3): n is 5-(N-3-phenylpropylcarboxyamide) uracil
  misc_feature (5)..(5): n is 5-(N-3-phenylpropylcarboxyamide) uracil
  misc_feature (7)..(7): n is 5-(N-3-phenylpropylcarboxyamide) uracil
  misc_feature (10)..(10): n is 5-(N-3-phenylpropylcarboxyamide) uracil
  misc_feature (12)..(12): n is 5-(N-3-phenylpropylcarboxyamide) uracil
  misc_feature (14)..(17): n is 5-(N-3-phenylpropylcarboxyamide) uracil
  misc_feature (20)..(20): n is 5-(N-3-phenylpropylcarboxyamide) uracil
  misc_feature (22)..(22): n is 5-(N-3-phenylpropylcarboxyamide) uracil
  misc_feature (26)..(28): n is 5-(N-3-phenylpropylcarboxyamide) uracil
  misc_feature (31)..(31): n is 5-(N-3-phenylpropylcarboxyamide) uracil
  misc_feature (34)..(34): n is 5-(N-3-phenylpropylcarboxyamide) uracil
tcngngncangngnnnnaangncagnnnaanganga

SEQ ID NO: 17 - DNA, 50 bp, Artificial Sequence
  Synthetic
  misc_feature (15)..(15): n is 5-(N-3-phenylpropylcarboxyamide) uracil
  misc_feature (17)..(17): n is 5-(N-3-phenylpropylcarboxyamide) uracil
  misc_feature (19)..(19): n is 5-(N-3-phenylpropylcarboxyamide) uracil
  misc_feature (22)..(23): n is 5-(N-3-phenylpropylcarboxyamide) uracil
  misc_feature (26)..(26): n is 5-(N-3-phenylpropylcarboxyamide) uracil
  misc_feature (28)..(28): n is 5-(N-3-phenylpropylcarboxyamide) uracil
  misc_feature (30)..(31): n is 5-(N-3-phenylpropylcarboxyamide) uracil
  misc_feature (33)..(33): n is 5-(N-3-phenylpropylcarboxyamide) uracil
  misc_feature (41)..(44): n is 5-(N-3-phenylpropylcarboxyamide) uracil
atctccgggaacacngncngannaangncnngnaagcgggnnnncggcag

SEQ ID NO: 18 - DNA, 50 bp, Artificial Sequence
  Synthetic
  misc_feature (6)..(6): n is 5-(N-3-phenylpropylcarboxyamide) uracil
  misc_feature (8)..(8): n is 5-(N-3-phenylpropylcarboxyamide) uracil
  misc_feature (10)..(10): n is 5-(N-3-phenylpropylcarboxyamide) uracil
  misc_feature (13)..(13): n is 5-(N-3-phenylpropylcarboxyamide) uracil
  misc_feature (15)..(15): n is 5-(N-3-phenylpropylcarboxyamide) uracil
  misc_feature (17)..(19): n is 5-(N-3-phenylpropylcarboxyamide) uracil
  misc_feature (22)..(22): n is 5-(N-3-phenylpropylcarboxyamide) uracil
  misc_feature (24)..(24): n is 5-(N-3-phenylpropylcarboxyamide) uracil
  misc_feature (30)..(31): n is 5-(N-3-phenylpropylcarboxyamide) uracil
  misc_feature (35)..(35): n is 5-(N-3-phenylpropylcarboxyamide) uracil
  misc_feature (38)..(38): n is 5-(N-3-phenylpropylcarboxyamide) uracil
  misc_feature (44)..(44): n is 5-(N-3-phenylpropylcarboxyamide) uracil
atctcngngncangngnnnaangncaaggnncaangangaggangggcag

SEQ ID NO: 19 - DNA, 50 bp, Artificial Sequence
  Synthetic
  misc_feature (6)..(8): n is 5-(N-3-phenylpropylcarboxyamide) uracil
  misc_feature (10)..(10): n is 5-(N-3-phenylpropylcarboxyamide) uracil
  misc_feature (12)..(12): n is 5-(N-3-phenylpropylcarboxyamide) uracil
  misc_feature (15)..(17): n is 5-(N-3-phenylpropylcarboxyamide) uracil
  misc_feature (19)..(19): n is 5-(N-3-phenylpropylcarboxyamide) uracil
  misc_feature (21)..(24): n is 5-(N-3-phenylpropylcarboxyamide) uracil
  misc_feature (26)..(26): n is 5-(N-3-phenylpropylcarboxyamide) uracil
  misc_feature (28)..(29): n is 5-(N-3-phenylpropylcarboxyamide) uracil
  misc_feature (31)..(31): n is 5-(N-3-phenylpropylcarboxyamide) uracil
  misc_feature (33)..(33): n is 5-(N-3-phenylpropylcarboxyamide) uracil
  misc_feature (35)..(35): n is 5-(N-3-phenylpropylcarboxyamide) uracil
  misc_feature (38)..(40): n is 5-(N-3-phenylpropylcarboxyamide) uracil
  misc_feature (42)..(42): n is 5-(N-3-phenylpropylcarboxyamide) uracil
  misc_feature (45)..(45): n is 5-(N-3-phenylpropylcarboxyamide) uracil
atctcnnngngnccnnngngnnnngngnncncngnccnnngnacnggcag

SEQ ID NO: 20 - DNA, 50 bp, Artificial Sequence
  Synthetic
  misc_feature (7)..(9): n is 5-(N-3-phenylpropylcarboxyamide) uracil
  misc_feature (11)..(11): n is 5-(N-3-phenylpropylcarboxyamide) uracil
  misc_feature (13)..(13): n is 5-(N-3-phenylpropylcarboxyamide) uracil
  misc_feature (15)..(15): n is 5-(N-3-phenylpropylcarboxyamide) uracil
  misc_feature (17)..(17): n is 5-(N-3-phenylpropylcarboxyamide) uracil
  misc_feature (19)..(20): n is 5-(N-3-phenylpropylcarboxyamide) uracil
  misc_feature (22)..(22): n is 5-(N-3-phenylpropylcarboxyamide) uracil
  misc_feature (26)..(26): n is 5-(N-3-phenylpropylcarboxyamide) uracil
  misc_feature (28)..(28): n is 5-(N-3-phenylpropylcarboxyamide) uracil
  misc_feature (30)..(31): n is 5-(N-3-phenylpropylcarboxyamide) uracil
  misc_feature (34)..(35): n is 5-(N-3-phenylpropylcarboxyamide) uracil
  misc_feature (38)..(39): n is 5-(N-3-phenylpropylcarboxyamide) uracil
atctccnnngncnangnanngngggngngnnccnngcnngaaacaggcag

SEQ ID NO: 21 - DNA, 50 bp, Artificial Sequence
  Synthetic
  misc_feature (13)..(13): n is 5-(N-3-phenylpropylcarboxyamide) uracil
  misc_feature (15)..(15): n is 5-(N-3-phenylpropylcarboxyamide) uracil
  misc_feature (18)..(19): n is 5-(N-3-phenylpropylcarboxyamide) uracil
  misc_feature (21)..(21): n is 5-(N-3-phenylpropylcarboxyamide) uracil
  misc_feature (23)..(23): n is 5-(N-3-phenylpropylcarboxyamide) uracil
  misc_feature (25)..(27): n is 5-(N-3-phenylpropylcarboxyamide) uracil
  misc_feature (29)..(30): n is 5-(N-3-phenylpropylcarboxyamide) uracil
  misc_feature (32)..(32): n is 5-(N-3-phenylpropylcarboxyamide) uracil
  misc_feature (34)..(35): n is 5-(N-3-phenylpropylcarboxyamide) uracil
  misc_feature (37)..(37): n is 5-(N-3-phenylpropylcarboxyamide) uracil
  misc_feature (43)..(43): n is 5-(N-3-phenylpropylcarboxyamide) uracil
atctcgagggcananagnngngncnnngnncngnngngggacngaggcag

SEQ ID NO: 22 - DNA, 50 bp, Artificial Sequence
  Synthetic
  misc_feature (6)..(8): n is 5-(N-3-phenylpropylcarboxyamide) uracil
  misc_feature (11)..(13): n is 5-(N-3-phenylpropylcarboxyamide) uracil
  misc_feature (15)..(16): n is 5-(N-3-phenylpropylcarboxyamide) uracil
  misc_feature (19)..(19): n is 5-(N-3-phenylpropylcarboxyamide) uracil
  misc_feature (23)..(23): n is 5-(N-3-phenylpropylcarboxyamide) uracil
  misc_feature (26)..(26): n is 5-(N-3-phenylpropylcarboxyamide) uracil
  misc_feature (28)..(28): n is 5-(N-3-phenylpropylcarboxyamide) uracil
  misc_feature (30)..(30): n is 5-(N-3-phenylpropylcarboxyamide) uracil
  misc_feature (32)..(33): n is 5-(N-3-phenylpropylcarboxyamide) uracil
  misc_feature (35)..(35): n is 5-(N-3-phenylpropylcarboxyamide) uracil
  misc_feature (37)..(37): n is 5-(N-3-phenylpropylcarboxyamide) uracil
  misc_feature (42)..(45): n is 5-(N-3-phenylpropylcarboxyamide) uracil
atctcnnnggnnngnnccncacncgngncnanngngnccccnnnnggcag

SEQ ID NO: 23 - DNA, 50 bp, Artificial Sequence
  Synthetic
  misc_feature (6)..(7): n is 5-(N-3-phenylpropylcarboxyamide) uracil
  misc_feature (10)..(10): n is 5-(N-3-phenylpropylcarboxyamide) uracil
  misc_feature (12)..(12): n is 5-(N-3-phenylpropylcarboxyamide) uracil
  misc_feature (14)..(14): n is 5-(N-3-phenylpropylcarboxyamide) uracil
  misc_feature (17)..(17): n is 5-(N-3-phenylpropylcarboxyamide) uracil
  misc_feature (19)..(20): n is 5-(N-3-phenylpropylcarboxyamide) uracil
  misc_feature (22)..(23): n is 5-(N-3-phenylpropylcarboxyamide) uracil
  misc_feature (25)..(27): n is 5-(N-3-phenylpropylcarboxyamide) uracil
  misc_feature (30)..(30): n is 5-(N-3-phenylpropylcarboxyamide) uracil
  misc_feature (32)..(32): n is 5-(N-3-phenylpropylcarboxyamide) uracil
  misc_feature (34)..(35): n is 5-(N-3-phenylpropylcarboxyamide) uracil
  misc_feature (40)..(42): n is 5-(N-3-phenylpropylcarboxyamide) uracil
  misc_feature (44)..(44): n is 5-(N-3-phenylpropylcarboxyamide) uracil
atctcnnacngngncgncnngnnannnacncngnnccgcnnngnaggcag

SEQ ID NO: 24 - DNA, 50 bp, Artificial Sequence
  Synthetic
  misc_feature (6)..(7): n is 5-(N-3-phenylpropylcarboxyamide) uracil
  misc_feature (12)..(12): n is 5-(N-3-phenylpropylcarboxyamide) uracil
  misc_feature (14)..(14): n is 5-(N-3-phenylpropylcarboxyamide) uracil
  misc_feature (18)..(18): n is 5-(N-3-phenylpropylcarboxyamide) uracil
  misc_feature (20)..(21): n is 5-(N-3-phenylpropylcarboxyamide) uracil
  misc_feature (23)..(23): n is 5-(N-3-phenylpropylcarboxyamide) uracil
  misc_feature (25)..(25): n is 5-(N-3-phenylpropylcarboxyamide) uracil
  misc_feature (27)..(28): n is 5-(N-3-phenylpropylcarboxyamide) uracil
  misc_feature (30)..(30): n is 5-(N-3-phenylpropylcarboxyamide) uracil
  misc_feature (32)..(33): n is 5-(N-3-phenylpropylcarboxyamide) uracil
  misc_feature (36)..(36): n is 5-(N-3-phenylpropylcarboxyamide) uracil
  misc_feature (38)..(38): n is 5-(N-3-phenylpropylcarboxyamide) uracil
  misc_feature (40)..(42): n is 5-(N-3-phenylpropylcarboxyamide) uracil
  misc_feature (44)..(45): n is 5-(N-3-phenylpropylcarboxyamide) uracil
atctcnncgacngngacngnncnancnngngnnccngngnnngnnggcag

SEQ ID NO: 25 - DNA, 50 bp, Artificial Sequence
  Synthetic
  misc_feature (7)..(8): n is 5-(N-3-phenylpropylcarboxyamide) uracil
  misc_feature (10)..(10): n is 5-(N-3-phenylpropylcarboxyamide) uracil
  misc_feature (13)..(13): n is 5-(N-3-phenylpropylcarboxyamide) uracil
  misc_feature (16)..(16): n is 5-(N-3-phenylpropylcarboxyamide) uracil
  misc_feature (18)..(20): n is 5-(N-3-phenylpropylcarboxyamide) uracil
  misc_feature (22)..(23): n is 5-(N-3-phenylpropylcarboxyamide) uracil
  misc_feature (25)..(25): n is 5-(N-3-phenylpropylcarboxyamide) uracil
  misc_feature (27)..(27): n is 5-(N-3-phenylpropylcarboxyamide) uracil
  misc_feature (30)..(31): n is 5-(N-3-phenylpropylcarboxyamide) uracil
  misc_feature (34)..(35): n is 5-(N-3-phenylpropylcarboxyamide) uracil
  misc_feature (37)..(37): n is 5-(N-3-phenylpropylcarboxyamide) uracil
  misc_feature (39)..(40): n is 5-(N-3-phenylpropylcarboxyamide) uracil
  misc_feature (42)..(42): n is 5-(N-3-phenylpropylcarboxyamide) uracil
  misc_feature (44)..(44): n is 5-(N-3-phenylpropylcarboxyamide) uracil
atctcanngngcnccngnnnanngngngcnngcnngncnngngnaggcag

SEQ ID NO: 26 - DNA, 50 bp, Artificial Sequence
  Synthetic
  misc_feature (6)..(6): n is 5-(N-3-phenylpropylcarboxyamide) uracil
  misc_feature (8)..(8): n is 5-(N-3-phenylpropylcarboxyamide) uracil
  misc_feature (10)..(10): n is 5-(N-3-phenylpropylcarboxyamide) uracil
  misc_feature (15)..(15): n is 5-(N-3-phenylpropylcarboxyamide) uracil
  misc_feature (19)..(20): n is 5-(N-3-phenylpropylcarboxyamide) uracil
  misc_feature (23)..(25): n is 5-(N-3-phenylpropylcarboxyamide) uracil
  misc_feature (27)..(27): n is 5-(N-3-phenylpropylcarboxyamide) uracil
  misc_feature (32)..(32): n is 5-(N-3-phenylpropylcarboxyamide) uracil
  misc_feature (34)..(34): n is 5-(N-3-phenylpropylcarboxyamide) uracil
  misc_feature (37)..(38): n is 5-(N-3-phenylpropylcarboxyamide) uracil
  misc_feature (43)..(45): n is 5-(N-3-phenylpropylcarboxyamide) uracil
atctcncncnaaaangccnngcnnngncgaangngcnnggaannnggcag

SEQ ID NO: 27 - DNA, 50 bp, Artificial Sequence
  Synthetic
  misc_feature (6)..(7): n is 5-(N-3-phenylpropylcarboxyamide) uracil
  misc_feature (9)..(9): n is 5-(N-3-phenylpropylcarboxyamide) uracil
  misc_feature (11)..(11): n is 5-(N-3-phenylpropylcarboxyamide) uracil
  misc_feature (13)..(13): n is 5-(N-3-phenylpropylcarboxyamide) uracil
  misc_feature (15)..(15): n is 5-(N-3-phenylpropylcarboxyamide) uracil
  misc_feature (18)..(18): n is 5-(N-3-phenylpropylcarboxyamide) uracil
  misc_feature (20)..(20): n is 5-(N-3-phenylpropylcarboxyamide) uracil
  misc_feature (22)..(22): n is 5-(N-3-phenylpropylcarboxyamide) uracil
  misc_feature (24)..(24): n is 5-(N-3-phenylpropylcarboxyamide) uracil
  misc_feature (26)..(26): n is 5-(N-3-phenylpropylcarboxyamide) uracil
  misc_feature (29)..(29): n is 5-(N-3-phenylpropylcarboxyamide) uracil
  misc_feature (31)..(33): n is 5-(N-3-phenylpropylcarboxyamide) uracil
  misc_feature (35)..(35): n is 5-(N-3-phenylpropylcarboxyamide) uracil
  misc_feature (37)..(37): n is 5-(N-3-phenylpropylcarboxyamide) uracil
  misc_feature (39)..(39): n is 5-(N-3-phenylpropylcarboxyamide) uracil
  misc_feature (41)..(41): n is 5-(N-3-phenylpropylcarboxyamide) uracil
atctcnngncnangnagngngngngnggncnnngnangngnacagggcag

SEQ ID NO: 28 - DNA, 50 bp, Artificial Sequence
  Synthetic
  misc_feature (14)..(14): n is 5-(N-3-phenylpropylcarboxyamide) uracil
  misc_feature (20)..(20): n is 5-(N-3-phenylpropylcarboxyamide) uracil
  misc_feature (22)..(22): n is 5-(N-3-phenylpropylcarboxyamide) uracil
  misc_feature (24)..(24): n is 5-(N-3-phenylpropylcarboxyamide) uracil
  misc_feature (26)..(28): n is 5-(N-3-phenylpropylcarboxyamide) uracil
  misc_feature (30)..(31): n is 5-(N-3-phenylpropylcarboxyamide) uracil
  misc_feature (33)..(33): n is 5-(N-3-phenylpropylcarboxyamide) uracil
  misc_feature (35)..(36): n is 5-(N-3-phenylpropylcarboxyamide) uracil
  misc_feature (38)..(38): n is 5-(N-3-phenylpropylcarboxyamide) uracil
  misc_feature (43)..(43): n is 5-(N-3-phenylpropylcarboxyamide) uracil
catctccgaggcanaccgangngnannngnncngnngnggacngaggcag

SEQ ID NO: 29 - DNA, 50 bp, Artificial Sequence
  Synthetic
  misc_feature (6)..(6): n is 5-(N-3-phenylpropylcarboxyamide) uracil
  misc_feature (8)..(10): n is 5-(N-3-phenylpropylcarboxyamide) uracil
  misc_feature (12)..(12): n is 5-(N-3-phenylpropylcarboxyamide) uracil
  misc_feature (14)..(14): n is 5-(N-3-phenylpropylcarboxyamide) uracil
  misc_feature (16)..(16): n is 5-(N-3-phenylpropylcarboxyamide) uracil
  misc_feature (18)..(18): n is 5-(N-3-phenylpropylcarboxyamide) uracil
  misc_feature (21)..(22): n is 5-(N-3-phenylpropylcarboxyamide) uracil
  misc_feature (24)..(24): n is 5-(N-3-phenylpropylcarboxyamide) uracil
  misc_feature (26)..(27): n is 5-(N-3-phenylpropylcarboxyamide) uracil
  misc_feature (31)..(31): n is 5-(N-3-phenylpropylcarboxyamide) uracil
  misc_feature (36)..(37): n is 5-(N-3-phenylpropylcarboxyamide) uracil
  misc_feature (39)..(41): n is 5-(N-3-phenylpropylcarboxyamide) uracil
  misc_feature (44)..(44): n is 5-(N-3-phenylpropylcarboxyamide) uracil
atctcnannncncngngncanngngnncgcncggcnngnnngancggcag

SEQ ID NO: 30 - DNA, 50 bp, Artificial Sequence
  Synthetic
  misc_feature (7)..(8): n is 5-(N-3-phenylpropylcarboxyamide) uracil
  misc_feature (10)..(10): n is 5-(N-3-phenylpropylcarboxyamide) uracil
  misc_feature (13)..(13): n is 5-(N-3-phenylpropylcarboxyamide) uracil
  misc_feature (15)..(15): n is 5-(N-3-phenylpropylcarboxyamide) uracil
  misc_feature (18)..(18): n is 5-(N-3-phenylpropylcarboxyamide) uracil
  misc_feature (20)..(20): n is 5-(N-3-phenylpropylcarboxyamide) uracil
  misc_feature (22)..(22): n is 5-(N-3-phenylpropylcarboxyamide) uracil
  misc_feature (24)..(25): n is 5-(N-3-phenylpropylcarboxyamide) uracil
  misc_feature (29)..(30): n is 5-(N-3-phenylpropylcarboxyamide) uracil
  misc_feature (34)..(35): n is 5-(N-3-phenylpropylcarboxyamide) uracil
  misc_feature (37)..(37): n is 5-(N-3-phenylpropylcarboxyamide) uracil
  misc_feature (39)..(41): n is 5-(N-3-phenylpropylcarboxyamide) uracil
  misc_feature (44)..(44): n is 5-(N-3-phenylpropylcarboxyamide) uracil
catctcnngnccncngangngncnngacnncacnngngnnngcnaggcag

SEQ ID NO: 31 - DNA, 50 bp, Artificial Sequence
  Synthetic
  misc_feature (8)..(10): n is 5-(N-3-phenylpropylcarboxyamide) uracil
  misc_feature (12)..(12): n is 5-(N-3-phenylpropylcarboxyamide) uracil
  misc_feature (14)..(15): n is 5-(N-3-phenylpropylcarboxyamide) uracil
  misc_feature (17)..(18): n is 5-(N-3-phenylpropylcarboxyamide) uracil
  misc_feature (24)..(25): n is 5-(N-3-phenylpropylcarboxyamide) uracil
  misc_feature (27)..(27): n is 5-(N-3-phenylpropylcarboxyamide) uracil
  misc_feature (29)..(30): n is 5-(N-3-phenylpropylcarboxyamide) uracil
  misc_feature (34)..(36): n is 5-(N-3-phenylpropylcarboxyamide) uracil
  misc_feature (38)..(38): n is 5-(N-3-phenylpropylcarboxyamide) uracil
  misc_feature (40)..(40): n is 5-(N-3-phenylpropylcarboxyamide) uracil
  misc_feature (43)..(45): n is 5-(N-3-phenylpropylcarboxyamide) uracil
atctcacnnngngnncnncacccnngncnnggcnnngncnccnnnggcag

SEQ ID NO: 32 - DNA, 50 bp, Artificial Sequence
  Synthetic
  misc_feature (7)..(7): n is 5-(N-3-phenylpropylcarboxyamide) uracil
  misc_feature (9)..(9): n is 5-(N-3-phenylpropylcarboxyamide) uracil
  misc_feature (12)..(16): n is 5-(N-3-phenylpropylcarboxyamide) uracil
  misc_feature (18)..(19): n is 5-(N-3-phenylpropylcarboxyamide) uracil
  misc_feature (21)..(21): n is 5-(N-3-phenylpropylcarboxyamide) uracil
  misc_feature (23)..(23): n is 5-(N-3-phenylpropylcarboxyamide) uracil
  misc_feature (25)..(25): n is 5-(N-3-phenylpropylcarboxyamide) uracil
  misc_feature (28)..(28): n is 5-(N-3-phenylpropylcarboxyamide) uracil
  misc_feature (31)..(31): n is 5-(N-3-phenylpropylcarboxyamide) uracil
  misc_feature (36)..(37): n is 5-(N-3-phenylpropylcarboxyamide) uracil
  misc_feature (39)..(39): n is 5-(N-3-phenylpropylcarboxyamide) uracil
  misc_feature (41)..(41): n is 5-(N-3-phenylpropylcarboxyamide) uracil
  misc_feature (43)..(43): n is 5-(N-3-phenylpropylcarboxyamide) uracil
  misc_feature (45)..(45): n is 5-(N-3-phenylpropylcarboxyamide) uracil
catctcnangcnnnnngnncngncnccncgncgacnncngncncnggcag

SEQ ID NO: 33 - DNA, 50 bp, Artificial Sequence
  Synthetic
  misc_feature (7)..(9): n is 5-(N-3-phenylpropylcarboxyamide) uracil
  misc_feature (12)..(12): n is 5-(N-3-phenylpropylcarboxyamide) uracil
  misc_feature (14)..(14): n is 5-(N-3-phenylpropylcarboxyamide) uracil
  misc_feature (16)..(17): n is 5-(N-3-phenylpropylcarboxyamide) uracil
  misc_feature (19)..(20): n is 5-(N-3-phenylpropylcarboxyamide) uracil
  misc_feature (22)..(23): n is 5-(N-3-phenylpropylcarboxyamide) uracil
  misc_feature (25)..(26): n is 5-(N-3-phenylpropylcarboxyamide) uracil
  misc_feature (31)..(32): n is 5-(N-3-phenylpropylcarboxyamide) uracil
  misc_feature (34)..(34): n is 5-(N-3-phenylpropylcarboxyamide) uracil
  misc_feature (37)..(37): n is 5-(N-3-phenylpropylcarboxyamide) uracil
  misc_feature (39)..(39): n is 5-(N-3-phenylpropylcarboxyamide) uracil
  misc_feature (41)..(41): n is 5-(N-3-phenylpropylcarboxyamide) uracil
  misc_feature (45)..(45): n is 5-(N-3-phenylpropylcarboxyamide) uracil
catctcnnncgngngnngnngnngnnacganngncgnangncggnggcag

SEQ ID NO: 34 - DNA, 50 bp, Artificial Sequence
  Synthetic
  misc_feature (10)..(10): n is 5-(N-3-phenylpropylcarboxyamide) uracil
  misc_feature (15)..(16): n is 5-(N-3-phenylpropylcarboxyamide) uracil
  misc_feature (25)..(25): n is 5-(N-3-phenylpropylcarboxyamide) uracil
  misc_feature (27)..(29): n is 5-(N-3-phenylpropylcarboxyamide) uracil
  misc_feature (32)..(35): n is 5-(N-3-phenylpropylcarboxyamide) uracil
  misc_feature (37)..(37): n is 5-(N-3-phenylpropylcarboxyamide) uracil
  misc_feature (39)..(39): n is 5-(N-3-phenylpropylcarboxyamide) uracil
  misc_feature (41)..(42): n is 5-(N-3-phenylpropylcarboxyamide) uracil
atctcagcgnagggnnaacggaacngnnnggnnnnangngnncacggcag

SEQ ID NO: 35 - DNA, 50 bp, Artificial Sequence
  Synthetic
  misc_feature (6)..(8): n is 5-(N-3-phenylpropylcarboxyamide) uracil
  misc_feature (12)..(12): n is 5-(N-3-phenylpropylcarboxyamide) uracil
  misc_feature (15)..(15): n is 5-(N-3-phenylpropylcarboxyamide) uracil
  misc_feature (17)..(18): n is 5-(N-3-phenylpropylcarboxyamide) uracil
  misc_feature (21)..(21): n is 5-(N-3-phenylpropylcarboxyamide) uracil
  misc_feature (24)..(25): n is 5-(N-3-phenylpropylcarboxyamide) uracil
  misc_feature (27)..(27): n is 5-(N-3-phenylpropylcarboxyamide) uracil
  misc_feature (29)..(30): n is 5-(N-3-phenylpropylcarboxyamide) uracil
  misc_feature (32)..(32): n is 5-(N-3-phenylpropylcarboxyamide) uracil
  misc_feature (34)..(36): n is 5-(N-3-phenylpropylcarboxyamide) uracil
  misc_feature (40)..(41): n is 5-(N-3-phenylpropylcarboxyamide) uracil
  misc_feature (44)..(45): n is 5-(N-3-phenylpropylcarboxyamide) uracil
atctcnnnggancangnnacnccnnangnngngnnnagcnngcnnggcag

SEQ ID NO: 36 - DNA, 50 bp, Artificial Sequence
  Synthetic
  misc_feature (7)..(10): n is 5-(N-3-phenylpropylcarboxyamide) uracil
  misc_feature (12)..(12): n is 5-(N-3-phenylpropylcarboxyamide) uracil
  misc_feature (14)..(14): n is 5-(N-3-phenylpropylcarboxyamide) uracil
  misc_feature (21)..(22): n is 5-(N-3-phenylpropylcarboxyamide) uracil
  misc_feature (25)..(25): n is 5-(N-3-phenylpropylcarboxyamide) uracil
  misc_feature (27)..(27): n is 5-(N-3-phenylpropylcarboxyamide) uracil
  misc_feature (29)..(30): n is 5-(N-3-phenylpropylcarboxyamide) uracil
  misc_feature (32)..(32): n is 5-(N-3-phenylpropylcarboxyamide) uracil
  misc_feature (34)..(35): n is 5-(N-3-phenylpropylcarboxyamide) uracil
  misc_feature (38)..(38): n is 5-(N-3-phenylpropylcarboxyamide) uracil
  misc_feature (41)..(41): n is 5-(N-3-phenylpropylcarboxyamide) uracil
  misc_feature (43)..(43): n is 5-(N-3-phenylpropylcarboxyamide) uracil
  misc_feature (45)..(45): n is 5-(N-3-phenylpropylcarboxyamide) uracil
atctccnnnngnanagggganncgnangnngngnnccngangnanggcag

SEQ ID NO: 37 - DNA, 50 bp, Artificial Sequence
  Synthetic
  misc_feature (9)..(11): n is 5-(N-3-phenylpropylcarboxyamide) uracil
  misc_feature (15)..(16): n is 5-(N-3-phenylpropylcarboxyamide) uracil
  misc_feature (18)..(18): n is 5-(N-3-phenylpropylcarboxyamide) uracil
  misc_feature (21)..(22): n is 5-(N-3-phenylpropylcarboxyamide) uracil
  misc_feature (24)..(25): n is 5-(N-3-phenylpropylcarboxyamide) uracil
  misc_feature (27)..(27): n is 5-(N-3-phenylpropylcarboxyamide) uracil
  misc_feature (29)..(30): n is 5-(N-3-phenylpropylcarboxyamide) uracil
  misc_feature (34)..(35): n is 5-(N-3-phenylpropylcarboxyamide) uracil
  misc_feature (37)..(37): n is 5-(N-3-phenylpropylcarboxyamide) uracil
  misc_feature (40)..(40): n is 5-(N-3-phenylpropylcarboxyamide) uracil
cccatctcnnnagcnngnganngnngnannacgnngnggnccaggcagac

SEQ ID NO: 38 - DNA, 50 bp, Artificial Sequence
  Synthetic
  misc_feature (6)..(7): n is 5-(N-3-phenylpropylcarboxyamide) uracil
  misc_feature (9)..(9): n is 5-(N-3-phenylpropylcarboxyamide) uracil
  misc_feature (12)..(13): n is 5-(N-3-phenylpropylcarboxyamide) uracil
  misc_feature (15)..(16): n is 5-(N-3-phenylpropylcarboxyamide) uracil
  misc_feature (18)..(19): n is 5-(N-3-phenylpropylcarboxyamide) uracil
  misc_feature (21)..(21): n is 5-(N-3-phenylpropylcarboxyamide) uracil
  misc_feature (25)..(26): n is 5-(N-3-phenylpropylcarboxyamide) uracil
  misc_feature (28)..(29): n is 5-(N-3-phenylpropylcarboxyamide) uracil
  misc_feature (32)..(32): n is 5-(N-3-phenylpropylcarboxyamide) uracil
  misc_feature (36)..(38): n is 5-(N-3-phenylpropylcarboxyamide) uracil
  misc_feature (40)..(41): n is 5-(N-3-phenylpropylcarboxyamide) uracil
  misc_feature (45)..(45): n is 5-(N-3-phenylpropylcarboxyamide) uracil
tctccnncnccnngnngnncnccanngnncgncgcnnngnnaccncggca

SEQ ID NO: 39 - DNA, 50 bp, Artificial Sequence
  Synthetic
  misc_feature (6)..(10): n is 5-(N-3-phenylpropylcarboxyamide) uracil
  misc_feature (12)..(12): n is 5-(N-3-phenylpropylcarboxyamide) uracil
  misc_feature (15)..(15): n is 5-(N-3-phenylpropylcarboxyamide) uracil
  misc_feature (19)..(19): n is 5-(N-3-phenylpropylcarboxyamide) uracil
  misc_feature (22)..(23): n is 5-(N-3-phenylpropylcarboxyamide) uracil
  misc_feature (27)..(27): n is 5-(N-3-phenylpropylcarboxyamide) uracil
  misc_feature (29)..(29): n is 5-(N-3-phenylpropylcarboxyamide) uracil
  misc_feature (31)..(31): n is 5-(N-3-phenylpropylcarboxyamide) uracil
  misc_feature (33)..(33): n is 5-(N-3-phenylpropylcarboxyamide) uracil
  misc_feature (36)..(36): n is 5-(N-3-phenylpropylcarboxyamide) uracil
  misc_feature (38)..(38): n is 5-(N-3-phenylpropylcarboxyamide) uracil
  misc_feature (40)..(40): n is 5-(N-3-phenylpropylcarboxyamide) uracil
  misc_feature (45)..(45): n is 5-(N-3-phenylpropylcarboxyamide) uracil
atctcnnnnnanccngcgnacnnccgncngngnccnancnccacnggcag

SEQ ID NO: 40 - DNA, 50 bp, Artificial Sequence
  Synthetic
  misc_feature (6)..(7): n is 5-(N-3-phenylpropylcarboxyamide) uracil
  misc_feature (10)..(11): n is 5-(N-3-phenylpropylcarboxyamide) uracil
  misc_feature (18)..(20): n is 5-(N-3-phenylpropylcarboxyamide) uracil
  misc_feature (23)..(23): n is 5-(N-3-phenylpropylcarboxyamide) uracil
  misc_feature (25)..(25): n is 5-(N-3-phenylpropylcarboxyamide) uracil
  misc_feature (27)..(27): n is 5-(N-3-phenylpropylcarboxyamide) uracil
  misc_feature (30)..(31): n is 5-(N-3-phenylpropylcarboxyamide) uracil
  misc_feature (33)..(33): n is 5-(N-3-phenylpropylcarboxyamide) uracil
  misc_feature (37)..(38): n is 5-(N-3-phenylpropylcarboxyamide) uracil
  misc_feature (40)..(41): n is 5-(N-3-phenylpropylcarboxyamide) uracil
  misc_feature (44)..(45): n is 5-(N-3-phenylpropylcarboxyamide) uracil
atctcnngannccgaccnnngangngncgnncnggcnnannccnnggcag

SEQ ID NO: 41 - DNA, 50 bp, Artificial Sequence
  Synthetic
  misc_feature (6)..(6): n is 5-(N-3-phenylpropylcarboxyamide) uracil
  misc_feature (8)..(8): n is 5-(N-3-phenylpropylcarboxyamide) uracil
  misc_feature (15)..(20): n is 5-(N-3-phenylpropylcarboxyamide) uracil
  misc_feature (22)..(22): n is 5-(N-3-phenylpropylcarboxyamide) uracil
  misc_feature (24)..(24): n is 5-(N-3-phenylpropylcarboxyamide) uracil
  misc_feature (27)..(27): n is 5-(N-3-phenylpropylcarboxyamide) uracil
  misc_feature (29)..(29): n is 5-(N-3-phenylpropylcarboxyamide) uracil
  misc_feature (31)..(31): n is 5-(N-3-phenylpropylcarboxyamide) uracil
  misc_feature (33)..(33): n is 5-(N-3-phenylpropylcarboxyamide) uracil
  misc_feature (38)..(38): n is 5-(N-3-phenylpropylcarboxyamide) uracil
  misc_feature (44)..(45): n is 5-(N-3-phenylpropylcarboxyamide) uracil
atctcnanccgcccnnnnnnancnccngngngnccgcngcaaannggcag

SEQ ID NO: 42 - DNA, 50 bp, Artificial Sequence
  Synthetic
  misc_feature (7)..(8): n is 5-(N-3-phenylpropylcarboxyamide) uracil
  misc_feature (10)..(10): n is 5-(N-3-phenylpropylcarboxyamide) uracil
  misc_feature (13)..(13): n is 5-(N-3-phenylpropylcarboxyamide) uracil
  misc_feature (15)..(15): n is 5-(N-3-phenylpropylcarboxyamide) uracil
  misc_feature (17)..(20): n is 5-(N-3-phenylpropylcarboxyamide) uracil
  misc_feature (22)..(22): n is 5-(N-3-phenylpropylcarboxyamide) uracil
  misc_feature (26)..(27): n is 5-(N-3-phenylpropylcarboxyamide) uracil
  misc_feature (30)..(30): n is 5-(N-3-phenylpropylcarboxyamide) uracil
  misc_feature (32)..(33): n is 5-(N-3-phenylpropylcarboxyamide) uracil
  misc_feature (35)..(35): n is 5-(N-3-phenylpropylcarboxyamide) uracil
  misc_feature (38)..(38): n is 5-(N-3-phenylpropylcarboxyamide) uracil
  misc_feature (40)..(41): n is 5-(N-3-phenylpropylcarboxyamide) uracil
catctcnncngcncnannnngncccnngcnanncngcncnnacccggcag

SEQ ID NO: 43 - DNA, 50 bp, Artificial Sequence
  Synthetic
  misc_feature (13)..(13): n is 5-(N-3-phenylpropylcarboxyamide) uracil
  misc_feature (16)..(17): n is 5-(N-3-phenylpropylcarboxyamide) uracil
  misc_feature (19)..(19): n is 5-(N-3-phenylpropylcarboxyamide) uracil
  misc_feature (21)..(21): n is 5-(N-3-phenylpropylcarboxyamide) uracil
  misc_feature (24)..(26): n is 5-(N-3-phenylpropylcarboxyamide) uracil
  misc_feature (29)..(29): n is 5-(N-3-phenylpropylcarboxyamide) uracil
  misc_feature (33)..(33): n is 5-(N-3-phenylpropylcarboxyamide) uracil
  misc_feature (36)..(36): n is 5-(N-3-phenylpropylcarboxyamide) uracil
  misc_feature (38)..(38): n is 5-(N-3-phenylpropylcarboxyamide) uracil
  misc_feature (44)..(45): n is 5-(N-3-phenylpropylcarboxyamide) uracil
catctcaacaccngcnngncncgnnncgncaanggnancggccnnggcag

SEQ ID NO: 44 - DNA, 50 bp, Artificial Sequence
  Synthetic
  misc_feature (6)..(6): n is 5-(N-3-phenylpropylcarboxyamide) uracil
  misc_feature (8)..(8): n is 5-(N-3-phenylpropylcarboxyamide) uracil
  misc_feature (10)..(10): n is 5-(N-3-phenylpropylcarboxyamide) uracil
  misc_feature (12)..(12): n is 5-(N-3-phenylpropylcarboxyamide) uracil
  misc_feature (15)..(16): n is 5-(N-3-phenylpropylcarboxyamide) uracil
  misc_feature (18)..(19): n is 5-(N-3-phenylpropylcarboxyamide) uracil
  misc_feature (22)..(23): n is 5-(N-3-phenylpropylcarboxyamide) uracil
  misc_feature (26)..(26): n is 5-(N-3-phenylpropylcarboxyamide) uracil
  misc_feature (29)..(29): n is 5-(N-3-phenylpropylcarboxyamide) uracil
  misc_feature (31)..(32): n is 5-(N-3-phenylpropylcarboxyamide) uracil
  misc_feature (35)..(35): n is 5-(N-3-phenylpropylcarboxyamide) uracil
  misc_feature (38)..(38): n is 5-(N-3-phenylpropylcarboxyamide) uracil
  misc_feature (40)..(40): n is 5-(N-3-phenylpropylcarboxyamide) uracil
  misc_feature (44)..(45): n is 5-(N-3-phenylpropylcarboxyamide) uracil
atctcnanangncgnngnnacnnccnccncnnacngancnacannaggca

SEQ ID NO: 45 - DNA, 50 bp, Artificial Sequence
  Synthetic
  misc_feature (6)..(8): n is 5-(N-3-phenylpropylcarboxyamide) uracil
  misc_feature (11)..(11): n is 5-(N-3-phenylpropylcarboxyamide) uracil
  misc_feature (13)..(13): n is 5-(N-3-phenylpropylcarboxyamide) uracil
  misc_feature (18)..(20): n is 5-(N-3-phenylpropylcarboxyamide) uracil
  misc_feature (22)..(22): n is 5-(N-3-phenylpropylcarboxyamide) uracil
  misc_feature (24)..(24): n is 5-(N-3-phenylpropylcarboxyamide) uracil
  misc_feature (26)..(26): n is 5-(N-3-phenylpropylcarboxyamide) uracil
  misc_feature (28)..(28): n is 5-(N-3-phenylpropylcarboxyamide) uracil
  misc_feature (30)..(31): n is 5-(N-3-phenylpropylcarboxyamide) uracil
  misc_feature (35)..(36): n is 5-(N-3-phenylpropylcarboxyamide) uracil
  misc_feature (39)..(39): n is 5-(N-3-phenylpropylcarboxyamide) uracil
  misc_feature (42)..(42): n is 5-(N-3-phenylpropylcarboxyamide) uracil
  misc_feature (44)..(45): n is 5-(N-3-phenylpropylcarboxyamide) uracil
atctcnnnggnanaagcnnnangngngncnnaggnnccncancnnggcag

SEQ ID NO: 46 - DNA, 50 bp, Artificial Sequence
  Synthetic
  misc_feature (7)..(7): n is 5-(N-3-phenylpropylcarboxyamide) uracil
  misc_feature (13)..(17): n is 5-(N-3-phenylpropylcarboxyamide) uracil
  misc_feature (19)..(19): n is 5-(N-3-phenylpropylcarboxyamide) uracil
  misc_feature (21)..(21): n is 5-(N-3-phenylpropylcarboxyamide) uracil
  misc_feature (25)..(27): n is 5-(N-3-phenylpropylcarboxyamide) uracil
  misc_feature (31)..(31): n is 5-(N-3-phenylpropylcarboxyamide) uracil
  misc_feature (33)..(34): n is 5-(N-3-phenylpropylcarboxyamide) uracil
  misc_feature (36)..(36): n is 5-(N-3-phenylpropylcarboxyamide) uracil
  misc_feature (38)..(38): n is 5-(N-3-phenylpropylcarboxyamide) uracil
  misc_feature (40)..(40): n is 5-(N-3-phenylpropylcarboxyamide) uracil
  misc_feature (43)..(44): n is 5-(N-3-phenylpropylcarboxyamide) uracil
atctcgnccaacnnnnncngncagnnngggncnngngngncgnngggcag

SEQ ID NO: 47 - DNA, 50 bp, Artificial Sequence
  Synthetic
  misc_feature (6)..(7): n is 5-(N-3-phenylpropylcarboxyamide) uracil
  misc_feature (9)..(9): n is 5-(N-3-phenylpropylcarboxyamide) uracil
  misc_feature (11)..(12): n is 5-(N-3-phenylpropylcarboxyamide) uracil
  misc_feature (14)..(14): n is 5-(N-3-phenylpropylcarboxyamide) uracil
  misc_feature (16)..(16): n is 5-(N-3-phenylpropylcarboxyamide) uracil
  misc_feature (18)..(18): n is 5-(N-3-phenylpropylcarboxyamide) uracil
  misc_feature (20)..(20): n is 5-(N-3-phenylpropylcarboxyamide) uracil
  misc_feature (23)..(23): n is 5-(N-3-phenylpropylcarboxyamide) uracil
  misc_feature (26)..(27): n is 5-(N-3-phenylpropylcarboxyamide) uracil
  misc_feature (30)..(30): n is 5-(N-3-phenylpropylcarboxyamide) uracil
  misc_feature (33)..(33): n is 5-(N-3-phenylpropylcarboxyamide) uracil
  misc_feature (39)..(41): n is 5-(N-3-phenylpropylcarboxyamide) uracil
  misc_feature (43)..(44): n is 5-(N-3-phenylpropylcarboxyamide) uracil
atctcnngncnngngngncnggnccnnccncancggccnnncnnaggcag

SEQ ID NO: 48 - skipped

SEQ ID NO: 49 - DNA, 6 bp, Artificial Sequence
  Synthetic
  misc_feature (1)..(1): n is a C-5 modified pyrimidine
  misc_feature (2)..(2): n is a C-5 modified pyrimidine, A or G
  misc_feature (3)..(3): n is a C-5 modified pyrimidine
  misc_feature (5)..(5): n is a C-5 modified pyrimidine or A
nnnsnv

SEQ ID NO: 50 - DNA, 6 bp, Artificial Sequence
  Synthetic
  misc_feature (1)..(1): n is a C-5 modified pyrimidine
  misc_feature (2)..(2): n is a C-5 modified pyrimidine, A or G
  misc_feature (3)..(3): n is a C-5 modified pyrimidine
  misc_feature (5)..(5): n is a C-5 modified pyrimidine, unmodified C, G or A
  misc_feature (6)..(6): n is a C-5 modified pyrimidine
nnnsnn

SEQ ID NO: 51 - DNA, 10 bp, Artificial Sequence
  Synthetic
  misc_feature (3)..(3): n is a C-5 modified pyrimidine
  misc_feature (6)..(6): n is a C-5 modified pyrimidine
aangangagg

SEQ ID NO: 52 - DNA, 35 bp, Artificial Sequence
  Synthetic
  misc_feature (1)..(1): n is a C-5 modified pyrimidine
  misc_feature (3)..(3): n is a C-5 modified pyrimidine
  misc_feature (5)..(5): n is a C-5 modified pyrimidine
  misc_feature (7)..(7): a may or may not be present
  misc_feature (8)..(8): n is a C-5 modified pyrimidine
  misc_feature (10)..(10): n is a C-5 modified pyrimidine
  misc_feature (12)..(14): n is a C-5 modified pyrimidine
  misc_feature (15)..(15): n is a C-5 modified pyrimidine or A
  misc_feature (17)..(17): n is a C-5 modified pyrimidine or A
  misc_feature (18)..(19): n is a C-5 modified pyrimidine or G
  misc_feature (20)..(20): c may or may not be present
  misc_feature (21)..(21): n is a C-5 modified pyrimidine or A
  misc_feature (24)..(25): n is a C-5 modified pyrimidine
  misc_feature (28)..(28): n is a C-5 modified pyrimidine
  misc_feature (31)..(31): n is a C-5 modified pyrimidine
ngngncangngnnnnannncnmgnnaangangagg

SEQ ID NO: 53 - protein, 925 aa, Artificial Sequence
  Synthetic
MTTEQRRSLQAFQDYIRKTLDPTYILSYMAPWFREEEVQYIQAEKNNKGPMEAATLFLKFLLELQEEGWFRGFLDALDHAGYSGLYEAIESWDFKKIEKLEEYRLLLKRLQPEFKTRIIPTDIISDLSECLINQECEEILQICSTKGMMAGAEKLVECLLRSDKENWPKTLKLALEKERNKFSELWIVEKGIKDVETEDLEDKMETSDIQIFYQEDPECQNLSENSCPPSEVSDTNLYSPFKPRNYQLELALPAMKGKNTIICAPTGCGKTFVSLLICEHHLKKFPQGQKGKVVFFANQIPVYEQQKSVFSKYFERHGYRVTGISGATAENVPVEQIVENNDIIILTPQILVNNLKKGTIPSLSIFTLMIFDECHNTSKQHPYNMIMFNYLDQKLGGSSGPLPQVIGLTASVGVGDAKNTDEALDYICKLCASLDASVIATVKHNLEELEQVVYKPQKFFRKVESRISDKFKYIIAQLMRDTESLAKRICKDLENLSQIQNREFGTQKYEQWIVTVQKACMVFQMPDKDEESRICKALFLYTSHLRKYNDALIISEHARMKDALDYLKDFFSNVRAAGFDEIEQDLTQRFEEKLQELESVSRDPSNENPKLEDLCFILQEEYHLNPETITILFVKTRALVDALKNWIEGNPKLSFLKPGILTGRGKTNQNTGMTLPAQKCILDAFKASGDHNILIATSVADEGIDIAQCNLVILYEYVGNVIKMIQTRGRGRARGSKCFLLTSNAGVIEKEQINMYKEKMMNDSILRLQTWDEAVFREKILHIQTHEKFIRDSQEKPKPVPDKENKKLLCRKCKALACYTADVRVIEECHYTVLGDAFKECFVSRPHPKPKQFSSFEKRAKIFCARQNCSHDWGIHVKYKTFEIPVIKIESFVVEDIATGVQTLYSKWKDFHFEKIPFDPAEMSK

SEQ ID NO: 54 - protein, 697 aa, Artificial Sequence
  Synthetic
PSEVSDTNLYSPFKPRNYQLELALPAMKGKNTIICAPTGCGKTFVSLLICEHHLKKFPQGQKGKVVFFANQIPVYEQQKSVFSKYFERHGYRVTGISGATAENVPVEQIVENNDIIILTPQILVNNLKKGTIPSLSIFTLMIFDECHNTSKQHPYNMIMFNYLDQKLGGSSGPLPQVIGLTASVGVGDAKNTDEALDYICKLCASLDASVIATVKHNLEELEQVVYKPQKFFRKVESRISDKFKYIIAQLMRDTESLAKRICKDLENLSQIQNREFGTQKYEQWIVTVQKACMVFQMPDKDEESRICKALFLYTSHLRKYNDALIISEHARMKDALDYLKDFFSNVRAAGFDEIEQDLTQRFEEKLQELESVSRDPSNENPKLEDLCFILQEEYHLNPETITILFVKTRALVDALKNWIEGNPKLSFLKPGILTGRGKTNQNTGMTLPAQKCILDAFKASGDHNILIATSVADEGIDIAQCNLVILYEYVGNVIKMIQTRGRGRARGSKCFLLTSNAGVIEKEQINMYKEKMMNDSILRLQTWDEAVFREKILHIQTHEKFIRDSQEKPKPVPDKENKKLLCRKCKALACYTADVRVIEECHYTVLGDAFKECFVSRPHPKPKQFSSFEKRAKIFCARQNCSHDWGIHVKYKTFEIPVIKIESFVVEDIATGVQTLYSKWKDFHFEKIPFDPAEMSK

SEQ ID NO: 55 - protein, 565 aa, Artificial Sequence
  Synthetic
PSEVSDTNLYSPFKPRNYQLELALPAMKGKNTIICAPTGCGKTFVSLLICEHHLKKFPQGQKGKVVFFANQIPVYEQQKSVFSKYFERHGYRVTGISGATAENVPVEQIVENNDIIILTPQILVNNLKKGTIPSLSIFTLMIFDECHNTSKQHPYNMIMFNYLDQKLGGSSGPLPQVIGLTASVGVGDAKNTDEALDYICKLCASLDASVIATVKHNLEELEQVVYKPQKFFRKVESRISDKFKYIIAQLMRDTESLAKRICKDLENLSQIQNREFGTQKYEQWIVTVQKACMVFQMPDKDEESRICKALFLYTSHLRKYNDALIISEHARMKDALDYLKDFFSNVRAAGFDEIEQDLTQRFEEKLQELESVSRDPSNENPKLEDLCFILQEEYHLNPETITILFVKTRALVDALKNWIEGNPKLSFLKPGILTGRGKTNQNTGMTLPAQKCILDAFKASGDHNILIATSVADEGIDIAQCNLVILYEYVGNVIKMIQTRGRGRARGSKCFLLTSNAGVIEKEQINMYKEKMMNDSILRLQTWDEAVFREKILHIQTHEKFIRDS

SEQ ID NO: 56 - protein, 134 aa, Artificial Sequence
  Synthetic
DSQEKPKPVPDKENKKLLCRKCKALACYTADVRVIEECHYTVLGDAFKECFVSRPHPKPKQFSSFEKRAKIFCARQNCSHDWGIHVKYKTFEIPVIKIESFVVEDIATGVQTLYSKWKDFHFEKIPFDPAEMSK

SEQ ID NO: 57 - protein, 925 aa, Artificial Sequence
  Synthetic
MTTEQRRSLQAFQDYIRKTLDPTYILSYMAPWFREEEVQYIQAEKNNKGPMEAATLFLKFLLELQEEGWFRGFLDALDHAGYSGLYEAIESWDFKKIEKLEEYRLLLKRLQPEFKTRIIPTDIISDLSECLINQECEEILQICSTKGMMAGAEKLVECLLRSDKENWPKTLKLALEKERNKFSELWIVEKGIKDVETEDLEDKMETSDIQIFYQEDPECQNLSENSCPPSEVSDTNLYSPFKPRNYQLELALPAMKGKNTIICAPTGCGKTFVSLLICEHHLKKFPQGQKGKVVFFANQIPVYEQQKSVFSKYFERHGYRVTGISGATAENVPVEQIVENNDIIILTPQILVNNLKKGTIPSLSIFTLMIFDECHNTSKQHPYNMIMFNYLDQKLGGSSGPLPQVIGLTASVGVGDAKNTDEALDYICKLCASLDASVIATVKHNLEELEQVVYKPQKFFRKVESRISDKFKYIIAQLMRDTESLAKRICKDLENLSQIQNREFGTQKYEQWIVTVQKACMVFQMPDKDEESRICKALFLYTSHLRKYNDALIISEHARMKDALDYLKDFFSNVRAAGFDEIEQDLTQRFEEKLQELESVSRDPSNENPKLEDLCFILQEEYHLNPETITILFVKTRALVDALKNWIEGNPKLSFLKPGILTGRGKTNQNTGMTLPAQKCILDAFKASGDHNILIATSVADEGIDIAQCNLVILYEYVGNVIKMIQTRGRGRARGSKCFLLTSNAGVIEKEQINMYKEKMMNDSILRLQTWDEAVFREKILHIQTHEKFIRDSQEKPKPVPDKENKKLLCRKCKALACYTADVRVIEECHYTVLGDAFKECFVSRPHPKPKQFSSFEKRAKIFCARQNCSHAWGIHVKYKTFEIPVIKIESFVVEDIATGVQTLYSKWKDFHFEKIPFDPAEMSK

SEQ ID NO: 58 - protein, 925 aa, Artificial Sequence
  Synthetic
MTTEQRRSLQAFQDYIRKTLDPTYILSYMAPWFREEEVQYIQAEKNNKGPMEAATLFLKFLLELQEEGWFRGFLDALDHAGYSGLYEAIESWDFKKIEKLEEYRLLLKRLQPEFKTRIIPTDIISDLSECLINQECEEILQICSTKGMMAGAEKLVECLLRSDKENWPKTLKLALEKERNKFSELWIVEKGIKDVETEDLEDKMETSDIQIFYQEDPECQNLSENSCPPSEVSDTNLYSPFKPRNYQLELALPAMKGKNTIICAPTGCGKTFVSLLICEHHLKKFPQGQKGKVVFFANQIPVYEQQKSVFSKYFERHGYRVTGISGATAENVPVEQIVENNDIIILTPQILVNNLKKGTIPSLSIFTLMIFDECHNTSKQHPYNMIMFNYLDQKLGGSSGPLPQVIGLTASVGVGDAKNTDEALDYICKLCASLDASVIATVKHNLEELEQVVYKPQKFFRKVESRISDKFKYIIAQLMRDTESLAKRICKDLENLSQIQNREFGTQKYEQWIVTVQKACMVFQMPDKDEESRICKALFLYTSHLRKYNDALIISEHARMKDALDYLKDFFSNVRAAGFDEIEQDLTQRFEEKLQELESVSRDPSNENPKLEDLCFILQEEYHLNPETITILFVKTRALVDALKNWIEGNPKLSFLKPGILTGRGKTAQNTGMTLPAQKCILDAFKASGDHNILIATSVADEGIDIAQCNLVILYEYVGNVIKMIQTRGRGRARGSKCFLLTSNAGVIEKEQINMYKEKMMNDSILRLQTWDEAVFREKILHIQTHEKFIRDSQEKPKPVPDKENKKLLCRKCKALACYTADVRVIEECHYTVLGDAFKECFVSRPHPKPKQFSSFEKRAKIFCARQNCSHDWGIHVKYKTFEIPVIKIESFVVEDIATGVQTLYSKWKDFHFEKIPFDPAEMSK

SEQ ID NO: 59 - protein, 925 aa, Artificial Sequence
  Synthetic
MTTEQRRSLQAFQDYIRKTLDPTYILSYMAPWFREEEVQYIQAEKNNKGPMEAATLFLKFLLELQEEGWFRGFLDALDHAGYSGLYEAIESWDFKKIEKLEEYRLLLKRLQPEFKTRIIPTDIISDLSECLINQECEEILQICSTKGMMAGAEKLVECLLRSDKENWPKTLKLALEKERNKFSELWIVEKGIKDVETEDLEDKMETSDIQIFYQEDPECQNLSENSCPPSEVSDTNLYSPFKPRNYQLELALPAMKGKNTIICAPTGCGKTFVSLLICEHHLKKFPQGQKGKVVFFANQIPVYEQQKSVFSKYFERHGYRVTGISGATAENVPVEQIVENNDIIILTPQILVNNLKKGTIPSLSIFTLMIFDECHNTSKQHPYNMIMFNYLDQKLGGSSGPLPQVIGLTASVGVGDAKNTDEALDYICKLCASLDASVIATVKHNLEELEQVVYKPQKFFRKVESRISDKFKYIIAQLMRDTESLAKRICKDLENLSQIQNREFGTQKYEQWIVTVQKACMVFQMPDKDEESRICKALFLYTSHLRKYNDALIISEHARMKDALDYLKDFFSNVRAAGFDEIEQDLTQRFEEKLQELESVSRDPSNENPKLEDLCFILQEEYHLNPETITILFVKTRALVDALKNWIEGNPKLSFLKPGILTGRGKTNQNTGMTLPAQKCILDAFKASGDHNILIATSVADEGIDIAQCNLVILYEYVGNVIKMIQTRGRGRARGSKCFLLTSNAGVIEKEQINMYKEKMMNDSILRLQTWDEAVFREKILHIQTHEKFIRDSQEKPKPVPDKENKKLLCRKCKALACYTADVRVIEECHYTVLGDAFKECFVSRPHPAPKQFSSFEKRAKIFCARQNCSHDWGIHVKYKTFEIPVIKIESFVVEDIATGVQTLYSKWKDFHFEKIPFDPAEMSK

SEQ ID NO: 60 - protein, 925 aa, Artificial Sequence
  Synthetic
MTTEQRRSLQAFQDYIRKTLDPTYILSYMAPWFREEEVQYIQAEKNNKGPMEAATLFLKFLLELQEEGWFRGFLDALDHAGYSGLYEAIESWDFKKIEKLEEYRLLLKRLQPEFKTRIIPTDIISDLSECLINQECEEILQICSTKGMMAGAEKLVECLLRSDKENWPKTLKLALEKERNKFSELWIVEKGIKDVETEDLEDKMETSDIQIFYQEDPECQNLSENSCPPSEVSDTNLYSPFKPRNYQLELALPAMKGKNTIICAPTGCGKTFVSLLICEHHLKKFPQGQKGKVVFFANQIPVYEQQKSVFSKYFERHGYRVTGISGATAENVPVEQIVENNDIIILTPQILVNNLKKGTIPSLSIFTLMIFDECHNTSKQHPYNMIMFNYLDQKLGGSSGPLPQVIGLTASVGVGDAKNTDEALDYICKLCASLDASVIATVKHNLEELEQVVYKPQKFFRKVESRISDKFKYIIAQLMRDTESLAKRICKDLENLSQIQNREFGTQKYEQWIVTVQKACMVFQMPDKDEESRICKALFLYTSHLRKYNDALIISEHARMKDALDYLKDFFSNVRAAGFDEIEQDLTQRFEEKLQELESVSRDPSNENPKLEDLCFILQEEYHLNPETITILFVKTRALVDALKNWIEGNPKLSFLKPGILTGRGKTNQNTGMTLPAQKCILDAFKASGDHNILIATSVADEGIDIAQCNLVILYEYVGNVIKMIQTRGRGRARGSKCFLLTSNAGVIEKEQINMYKEKMMNDSILRLQTWDEAVFREKILHIQTHEKFIRDSQEKPKPVPDKENKKLLCRKCKALACYTADVRVIEECHYTVLGDAFKECFVSRPHPKPKQFSSFEKRAKIFCARQNCSHDWGIHVKYKTFEIPVIAIESFVVEDIATGVQTLYSKWKDFHFEKIPFDPAEMSK

SEQ ID NO: 61 - protein, 925 aa, Artificial Sequence
  Synthetic
MTTEQRRSLQAFQDYIRKTLDPTYILSYMAPWFREEEVQYIQAEKNNKGPMEAATLFLKFLLELQEEGWFRGFLDALDHAGYSGLYEAIESWDFKKIEKLEEYRLLLKRLQPEFKTRIIPTDIISDLSECLINQECEEILQICSTKGMMAGAEKLVECLLRSDKENWPKTLKLALEKERNKFSELWIVEKGIKDVETEDLEDKMETSDIQIFYQEDPECQNLSENSCPPSEVSDTNLYSPFKPRNYQLELALPAMKGKNTIICAPTGCGKTFVSLLICEHHLKKFPQGQKGKVVFFANQIPVYEQQKSVFSKYFERHGYRVTGISGATAENVPVEQIVENNDIIILTPQILVNNLKKGTIPSLSIFTLMIFDECHNTSKQHPYNMIMFNYLDQKLGGSSGPLPQVIGLTASVGVGDAKNTDEALDYICKLCASLDASVIATVKHNLEELEQVVYKPQKFFRKVESRISDKFKYIIAQLMRDTESLAKRICKDLENLSQIQNREFGTQKYEQWIVTVQKACMVFQMPDKDEESRICKALFLYTSHLRKYNDALIISEHARMKDALDYLKDFFSNVRAAGFDEIEQDLTQRFEEKLQELESVSRDPSNENPKLEDLCFILQEEYHLNPETITILFVKTRALVDALKNWIEGNPKLSFLKPGILTGAGKTNQNTGMTLPAQKCILDAFKASGDHNILIATSVADEGIDIAQCNLVILYEYVGNVIKMIQTRGRGRARGSKCFLLTSNAGVIEKEQINMYKEKMMNDSILRLQTWDEAVFREKILHIQTHEKFIRDSQEKPKPVPDKENKKLLCRKCKALACYTADVRVIEECHYTVLGDAFKECFVSRPHPKPKQFSSFEKRAKIFCARQNCSHDWGIHVKYKTFEIPVIKIESFVVEDIATGVQTLYSKWKDFHFEKIPFDPAEMSK

SEQ ID NO: 62 - protein, 925 aa, Artificial Sequence
  Synthetic
MTTEQRRSLQAFQDYIRKTLDPTYILSYMAPWFREEEVQYIQAEKNNKGPMEAATLFLKFLLELQEEGWFRGFLDALDHAGYSGLYEAIESWDFKKIEKLEEYRLLLKRLQPEFKTRIIPTDIISDLSECLINQECEEILQICSTKGMMAGAEKLVECLLRSDKENWPKTLKLALEKERNKFSELWIVEKGIKDVETEDLEDKMETSDIQIFYQEDPECQNLSENSCPPSEVSDTNLYSPFKPRNYQLELALPAMKGKNTIICAPTGCGKTFVSLLICEHHLKKFPQGQKGKVVFFANQIPVYEQQKSVFSKYFERHGYRVTGISGATAENVPVEQIVENNDIIILTPQILVNNLKKGTIPSLSIFTLMIFDECHNTSKQHPYNMIMFNYLDQKLGGSSGPLPQVIGLTASVGVGDAKNTDEALDYICKLCASLDASVIATVKHNLEELEQVVYKPQKFFRKVESRISDKFKYIIAQLMRDTESLAKRICKDLENLSQIQNREFGTQKYEQWIVTVQKACMVFQMPDKDEESRICKALFLYTSHLRKYNDALIISEHARMKDALDYLKDFFSNVRAAGFDEIEQDLTQRFEEKLQELESVSRDPSNENPKLEDLCFILQEEYHLNPETITILFVKTRALVDALKNWIEGNPKLSFLKPGILTGRGKTNQNTGMTLPAQKCILDAFKASGDHNILIATSVADEGIDIAQCNLVILYEYVGNVIKMIQTRGRGRARGSKCFLLTSNAGVIEKEQINMYKEKMMNDSILRLQTWDEAVFREKILHIQTHEKFIRDSQEKPKPVPDKENKKLLCRKCKALACYTADVRVIEECHYTVLGDAFKECFVSRPAPKPKQFSSFEKRAKIFCARQNCSHDWGIHVKYKTFEIPVIKIESFVVEDIATGVQTLYSKWKDFHFEKIPFDPAEMSK

SEQ ID NO: 63 - protein, 925 aa, Artificial Sequence
  Synthetic
MTTEQRRSLQAFQDYIRKTLDPTYILSYMAPWFREEEVQYIQAEKNNKGPMEAATLFLKFLLELQEEGWFRGFLDALDHAGYSGLYEAIESWDFKKIEKLEEYRLLLKRLQPEFKTRIIPTDIISDLSECLINQECEEILQICSTKGMMAGAEKLVECLLRSDKENWPKTLKLALEKERNKFSELWIVEKGIKDVETEDLEDKMETSDIQIFYQEDPECQNLSENSCPPSEVSDTNLYSPFKPRNYQLELALPAMKGKNTIICAPTGCGKTFVSLLICEHHLKKFPQGQKGKVVFFANQIPVYEQQKSVFSKYFERHGYRVTGISGATAENVPVEQIVENNDIIILTPQILVNNLKKGTIPSLSIFTLMIFDECHNTSKQHPYNMIMFNYLDQKLGGSSGPLPQVIGLTASVGVGDAKNTDEALDYICKLCASLDASVIATVKHNLEELEQVVYKPQKFFRKVESRISDKFKYIIAQLMRDTESLAKRICKDLENLSQIQNREFGTQKYEQWIVTVQKACMVFQMPDKDEESRICKALFLYTSHLRKYNDALIISEHARMKDALDYLKDFFSNVRAAGFDEIEQDLTQRFEEKLQELESVSRDPSNENPKLEDLCFILQEEYHLNPETITILFVKTRALVDALKNWIEGNPKLSFLKPGILTGRGKTNQNTGMTLPAQKCILDAFKASGDHNILIATSVADEGIDIAQCNLVILYEYVGNVIKMIQTRGRGRARGSKCFLLTSNAGVIEKEQINMYKEKMMNDSILRLQTWDEAVFREKILHIQTHEKFIRDSQEKPKPVPDKENKKLLCRKCKALACYTADVRVIEECHYTVLGDAFKECFVSRPHPKPKQFSSFEARAKIFCARQNCSHDWGIHVKYKTFEIPVIKIESFVVEDIATGVQTLYSKWKDFHFEKIPFDPAEMSK

SEQ ID NO: 64 - protein, 925 aa, Artificial Sequence
  Synthetic
MTTEQRRSLQAFQDYIRKTLDPTYILSYMAPWFREEEVQYIQAEKNNKGPMEAATLFLKFLLELQEEGWFRGFLDALDHAGYSGLYEAIESWDFKKIEKLEEYRLLLKRLQPEFKTRIIPTDIISDLSECLINQECEEILQICSTKGMMAGAEKLVECLLRSDKENWPKTLKLALEKERNKFSELWIVEKGIKDVETEDLEDKMETSDIQIFYQEDPECQNLSENSCPPSEVSDTNLYSPFKPRNYQLELALPAMKGKNTIICAPTGCGKTFVSLLICEHHLKKFPQGQKGKVVFFANQIPVYEQQKSVFSKYFERHGYRVTGISGATAENVPVEQIVENNDIIILTPQILVNNLKKGTIPSLSIFTLMIFDECHNTSKQHPYNMIMFNYLDQKLGGSSGPLPQVIGLTASVGVGDAKNTDEALDYICKLCASLDASVIATVKHNLEELEQVVYKPQKFFRKVESRISDKFKYIIAQLMRDTESLAKRICKDLENLSQIQNREFGTQKYEQWIVTVQKACMVFQMPDKDEESRICKALFLYTSHLRKYNDALIISEHARMKDALDYLKDFFSNVRAAGFDEIEQDLTQRFEEKLQELESVSRDPSNENPKLEDLCFILQEEYHLNPETITILFVKTRALVDALKNWIEGNPKLSFLKPGILTGRGKTNQNTGMTLPAQKCILDAFKASGDHNILIATSVADEGIDIAQCNLVILYEYVGNVIKMIQTRGRGRARGSKCFLLTSNAGVIEKEQINMYKEKMMNDSILRLQTWDEAVFREKILHIQTHEKFIRDSQEKPKPVPDKENKKLLCRKCKALACYTADVRVIEECHYTVLGDAFKECFVSRPHPKPKQFSSFEKRAAIFCARQNCSHDWGIHVKYKTFEIPVIKIESFVVEDIATGVQTLYSKWKDFHFEKIPFDPAEMSK

SEQ ID NO: 65 - DNA, 6 bp, Artificial Sequence
  Synthetic
  misc_feature (1)..(1): n is a, c, g, or t
  misc_feature (3)..(3): n is a, c, g, or t
  misc_feature (5)..(5): n is a, c, g, or t
ngngnc

SEQ ID NO: 66 - DNA, 6 bp, Artificial Sequence
  Synthetic
  misc_feature (1)..(1): n is a, c, g, or t
  misc_feature (3)..(3): n is a, c, g, or t
  misc_feature (5)..(6): n is a, c, g, or t
ngngnn

SEQ ID NO: 67 - DNA, 10 bp, Artificial Sequence
  Synthetic
  misc_feature (3)..(3): n is a, c, g, or t
  misc_feature (6)..(6): n is a, c, g, or t
aangangagg

SEQ ID NO: 68 - DNA, 40 bp, Artificial Sequence
  Synthetic
  misc_feature (1)..(1): n is 5-(N-3-phenylpropylcarboxyamide) uracil
  misc_feature (3)..(3): n is 5-(N-3-phenylpropylcarboxyamide) uracil
  misc_feature (5)..(5): n is 5-(N-3-phenylpropylcarboxyamide) uracil
  misc_feature (8)..(8): n is 5-(N-3-phenylpropylcarboxyamide) uracil
  misc_feature (10)..(10): n is 5-(N-3-phenylpropylcarboxyamide) uracil
  misc_feature (12)..(15): n is 5-(N-3-phenylpropylcarboxyamide) uracil
  misc_feature (18)..(18): n is 5-(N-3-phenylpropylcarboxyamide) uracil
  misc_feature (20)..(20): n is 5-(N-3-phenylpropylcarboxyamide) uracil
  misc_feature (24)..(26): n is 5-(N-3-phenylpropylcarboxyamide) uracil
  misc_feature (29)..(29): n is 5-(N-3-phenylpropylcarboxyamide) uracil
  misc_feature (32)..(32): n is 5-(N-3-phenylpropylcarboxyamide) uracil
ngngncangngnnnnaangncagnnnaangangagggacg

SEQ ID NO: 69 - DNA, 40 bp, Artificial Sequence
  Synthetic
  misc_feature (1)..(1): n is 5-(N-3-phenylpropylcarboxyamide) uracil
  misc_feature (3)..(3): n is 5-(N-3-phenylpropylcarboxyamide) uracil
  misc_feature (5)..(5): n is 5-(N-3-phenylpropylcarboxyamide) uracil
  misc_feature (8)..(8): n is 5-(N-3-phenylpropylcarboxyamide) uracil
  misc_feature (10)..(10): n is 5-(N-3-phenylpropylcarboxyamide) uracil
  misc_feature (12)..(14): n is 5-(N-3-phenylpropylcarboxyamide) uracil
  misc_feature (17)..(17): n is 5-(N-3-phenylpropylcarboxyamide) uracil
  misc_feature (19)..(19): n is 5-(N-3-phenylpropylcarboxyamide) uracil
  misc_feature (25)..(26): n is 5-(N-3-phenylpropylcarboxyamide) uracil
  misc_feature (30)..(30): n is 5-(N-3-phenylpropylcarboxyamide) uracil
  misc_feature (33)..(33): n is 5-(N-3-phenylpropylcarboxyamide) uracil
  misc_feature (39)..(39): n is 5-(N-3-phenylpropylcarboxyamide) uracil
ngngncangngnnnaangncaaggnncaangangaggang

SEQ ID NO: 70 - DNA, 40 bp, Artificial Sequence
  Synthetic
  misc_feature (1)..(3): n is 5-(N-3-phenylpropylcarboxyamide) uracil
  misc_feature (5)..(5): n is 5-(N-3-phenylpropylcarboxyamide) uracil
  misc_feature (7)..(7): n is 5-(N-3-phenylpropylcarboxyamide) uracil
  misc_feature (10)..(12): n is 5-(N-3-phenylpropylcarboxyamide) uracil
  misc_feature (14)..(14): n is 5-(N-3-phenylpropylcarboxyamide) uracil
  misc_feature (16)..(19): n is 5-(N-3-phenylpropylcarboxyamide) uracil
  misc_feature (21)..(21): n is 5-(N-3-phenylpropylcarboxyamide) uracil
  misc_feature (23)..(24): n is 5-(N-3-phenylpropylcarboxyamide) uracil
  misc_feature (26)..(26): n is 5-(N-3-phenylpropylcarboxyamide) uracil
  misc_feature (28)..(28): n is 5-(N-3-phenylpropylcarboxyamide) uracil
  misc_feature (30)..(30): n is 5-(N-3-phenylpropylcarboxyamide) uracil
  misc_feature (33)..(35): n is 5-(N-3-phenylpropylcarboxyamide) uracil
  misc_feature (37)..(37): n is 5-(N-3-phenylpropylcarboxyamide) uracil
  misc_feature (40)..(40): n is 5-(N-3-phenylpropylcarboxyamide) uracil
nnngngnccnnngngnnnngngnncncngnccnnngnacn

SEQ ID NO: 71 - DNA, 40 bp, Artificial Sequence
  Synthetic
  misc_feature (2)..(4): n is 5-(N-3-phenylpropylcarboxyamide) uracil
  misc_feature (6)..(6): n is 5-(N-3-phenylpropylcarboxyamide) uracil
  misc_feature (8)..(8): n is 5-(N-3-phenylpropylcarboxyamide) uracil
  misc_feature (10)..(10): n is 5-(N-3-phenylpropylcarboxyamide) uracil
  misc_feature (12)..(12): n is 5-(N-3-phenylpropylcarboxyamide) uracil
  misc_feature (14)..(15): n is 5-(N-3-phenylpropylcarboxyamide) uracil
  misc_feature (17)..(17): n is 5-(N-3-phenylpropylcarboxyamide) uracil
  misc_feature (21)..(21): n is 5-(N-3-phenylpropylcarboxyamide) uracil
  misc_feature (23)..(23): n is 5-(N-3-phenylpropylcarboxyamide) uracil
  misc_feature (25)..(26): n is 5-(N-3-phenylpropylcarboxyamide) uracil
  misc_feature (29)..(30): n is 5-(N-3-phenylpropylcarboxyamide) uracil
  misc_feature (33)..(34): n is 5-(N-3-phenylpropylcarboxyamide) uracil
cnnngncnangnanngngggngngnnccnngcnngaaaca

SEQ ID NO: 72 - DNA, 40 bp, Artificial Sequence
  Synthetic
  misc_feature (8)..(8): n is 5-(N-3-phenylpropylcarboxyamide) uracil
  misc_feature (10)..(10): n is 5-(N-3-phenylpropylcarboxyamide) uracil
  misc_feature (13)..(14): n is 5-(N-3-phenylpropylcarboxyamide) uracil
  misc_feature (16)..(16): n is 5-(N-3-phenylpropylcarboxyamide) uracil
  misc_feature (18)..(18): n is 5-(N-3-phenylpropylcarboxyamide) uracil
  misc_feature (20)..(22): n is 5-(N-3-phenylpropylcarboxyamide) uracil
  misc_feature (24)..(25): n is 5-(N-3-phenylpropylcarboxyamide) uracil
  misc_feature (27)..(27): n is 5-(N-3-phenylpropylcarboxyamide) uracil
  misc_feature (29)..(30): n is 5-(N-3-phenylpropylcarboxyamide) uracil
  misc_feature (32)..(32): n is 5-(N-3-phenylpropylcarboxyamide) uracil
  misc_feature (38)..(38): n is 5-(N-3-phenylpropylcarboxyamide) uracil
gagggcananagnngngncnnngnncngnngngggacnga

SEQ ID NO: 73 - DNA, 40 bp, Artificial Sequence
  Synthetic
  misc_feature (1)..(3): n is 5-(N-3-phenylpropylcarboxyamide) uracil
  misc_feature (6)..(8): n is 5-(N-3-phenylpropylcarboxyamide) uracil
  misc_feature (10)..(11): n is 5-(N-3-phenylpropylcarboxyamide) uracil
  misc_feature (14)..(14): n is 5-(N-3-phenylpropylcarboxyamide) uracil
  misc_feature (18)..(18): n is 5-(N-3-phenylpropylcarboxyamide) uracil
  misc_feature (21)..(21): n is 5-(N-3-phenylpropylcarboxyamide) uracil
  misc_feature (23)..(23): n is 5-(N-3-phenylpropylcarboxyamide) uracil
  misc_feature (25)..(25): n is 5-(N-3-phenylpropylcarboxyamide) uracil
  misc_feature (27)..(28): n is 5-(N-3-phenylpropylcarboxyamide) uracil
  misc_feature (30)..(30): n is 5-(N-3-phenylpropylcarboxyamide) uracil
  misc_feature (32)..(32): n is 5-(N-3-phenylpropylcarboxyamide) uracil
  misc_feature (37)..(40): n is 5-(N-3-phenylpropylcarboxyamide) uracil
nnnggnnngnnccncacncgngncnanngngnccccnnnn

SEQ ID NO: 74 - DNA, 40 bp, Artificial Sequence
  Synthetic
  misc_feature (1)..(2): n is 5-(N-3-phenylpropylcarboxyamide) uracil
  misc_feature (5)..(5): n is 5-(N-3-phenylpropylcarboxyamide) uracil
  misc_feature (7)..(7): n is 5-(N-3-phenylpropylcarboxyamide) uracil
  misc_feature (9)..(9): n is 5-(N-3-phenylpropylcarboxyamide) uracil
  misc_feature (12)..(12): n is 5-(N-3-phenylpropylcarboxyamide) uracil
  misc_feature (14)..(15): n is 5-(N-3-phenylpropylcarboxyamide) uracil
  misc_feature (17)..(18): n is 5-(N-3-phenylpropylcarboxyamide) uracil
  misc_feature (20)..(22): n is 5-(N-3-phenylpropylcarboxyamide) uracil
  misc_feature (25)..(25): n is 5-(N-3-phenylpropylcarboxyamide) uracil
  misc_feature (27)..(27): n is 5-(N-3-phenylpropylcarboxyamide) uracil
  misc_feature (29)..(30): n is 5-(N-3-phenylpropylcarboxyamide) uracil
  misc_feature (35)..(37): n is 5-(N-3-phenylpropylcarboxyamide) uracil
  misc_feature (39)..(39): n is 5-(N-3-phenylpropylcarboxyamide) uracil
nnacngngncgncnngnnannnacncngnnccgcnnngna

SEQ ID NO: 75 - DNA, 40 bp, Artificial Sequence
  Synthetic
  misc_feature (1)..(2): n is 5-(N-3-phenylpropylcarboxyamide) uracil
  misc_feature (7)..(7): n is 5-(N-3-phenylpropylcarboxyamide) uracil
  misc_feature (9)..(9): n is 5-(N-3-phenylpropylcarboxyamide) uracil
  misc_feature (13)..(13): n is 5-(N-3-phenylpropylcarboxyamide) uracil
  misc_feature (15)..(16): n is 5-(N-3-phenylpropylcarboxyamide) uracil
  misc_feature (18)..(18): n is 5-(N-3-phenylpropylcarboxyamide) uracil
  misc_feature (20)..(20): n is 5-(N-3-phenylpropylcarboxyamide) uracil
  misc_feature (22)..(23): n is 5-(N-3-phenylpropylcarboxyamide) uracil
  misc_feature (25)..(25): n is 5-(N-3-phenylpropylcarboxyamide) uracil
  misc_feature (27)..(28): n is 5-(N-3-phenylpropylcarboxyamide) uracil
  misc_feature (31)..(31): n is 5-(N-3-phenylpropylcarboxyamide) uracil
  misc_feature (33)..(33): n is 5-(N-3-phenylpropylcarboxyamide) uracil
  misc_feature (35)..(37): n is 5-(N-3-phenylpropylcarboxyamide) uracil
  misc_feature (39)..(40): n is 5-(N-3-phenylpropylcarboxyamide) uracil
nncgacngngacngnncnancnngngnnccngngnnngnn

SEQ ID NO: 76 - DNA, 40 bp, Artificial Sequence
  Synthetic
  misc_feature (2)..(3): n is 5-(N-3-phenylpropylcarboxyamide) uracil
  misc_feature (5)..(5): n is 5-(N-3-phenylpropylcarboxyamide) uracil
  misc_feature (8)..(8): n is 5-(N-3-phenylpropylcarboxyamide) uracil
  misc_feature (11)..(11): n is 5-(N-3-phenylpropylcarboxyamide) uracil
  misc_feature (13)..(15): n is 5-(N-3-phenylpropylcarboxyamide) uracil
  misc_feature (17)..(18): n is 5-(N-3-phenylpropylcarboxyamide) uracil
  misc_feature (20)..(20): n is 5-(N-3-phenylpropylcarboxyamide) uracil
  misc_feature (22)..(22): n is 5-(N-3-phenylpropylcarboxyamide) uracil
  misc_feature (25)..(26): n is 5-(N-3-phenylpropylcarboxyamide) uracil
  misc_feature (29)..(30): n is 5-(N-3-phenylpropylcarboxyamide) uracil
  misc_feature (32)..(32): n is 5-(N-3-phenylpropylcarboxyamide) uracil
  misc_feature (34)..(35): n is 5-(N-3-phenylpropylcarboxyamide) uracil
  misc_feature (37)..(37): n is 5-(N-3-phenylpropylcarboxyamide) uracil
  misc_feature (39)..(39): n is 5-(N-3-phenylpropylcarboxyamide) uracil
anngngcnccngnnnanngngngcnngcnngncnngngna

SEQ ID NO: 77 - DNA, 40 bp, Artificial Sequence
  Synthetic
  misc_feature (1)..(1): n is 5-(N-3-phenylpropylcarboxyamide) uracil
  misc_feature (3)..(3): n is 5-(N-3-phenylpropylcarboxyamide) uracil
  misc_feature (5)..(5): n is 5-(N-3-phenylpropylcarboxyamide) uracil
  misc_feature (10)..(10): n is 5-(N-3-phenylpropylcarboxyamide) uracil
  misc_feature (14)..(15): n is 5-(N-3-phenylpropylcarboxyamide) uracil
  misc_feature (18)..(20): n is 5-(N-3-phenylpropylcarboxyamide) uracil
  misc_feature (22)..(22): n is 5-(N-3-phenylpropylcarboxyamide) uracil
  misc_feature (27)..(27): n is 5-(N-3-phenylpropylcarboxyamide) uracil
  misc_feature (29)..(29): n is 5-(N-3-phenylpropylcarboxyamide) uracil
  misc_feature (32)..(33): n is 5-(N-3-phenylpropylcarboxyamide) uracil
  misc_feature (38)..(40): n is 5-(N-3-phenylpropylcarboxyamide) uracil
ncncnaaaangccnngcnnngncgaangngcnnggaannn

SEQ ID NO: 78 - DNA, 40 bp, Artificial Sequence
  Synthetic
  misc_feature (1)..(2): n is 5-(N-3-phenylpropylcarboxyamide) uracil
  misc_feature (4)..(4): n is 5-(N-3-phenylpropylcarboxyamide) uracil
  misc_feature (6)..(6): n is 5-(N-3-phenylpropylcarboxyamide) uracil
  misc_feature (8)..(8): n is 5-(N-3-phenylpropylcarboxyamide) uracil
  misc_feature (10)..(10): n is 5-(N-3-phenylpropylcarboxyamide) uracil
  misc_feature (13)..(13): n is 5-(N-3-phenylpropylcarboxyamide) uracil
  misc_feature (15)..(15): n is 5-(N-3-phenylpropylcarboxyamide) uracil
  misc_feature (17)..(17): n is 5-(N-3-phenylpropylcarboxyamide) uracil
  misc_feature (19)..(19): n is 5-(N-3-phenylpropylcarboxyamide) uracil
  misc_feature (21)..(21): n is 5-(N-3-phenylpropylcarboxyamide) uracil
  misc_feature (24)..(24): n is 5-(N-3-phenylpropylcarboxyamide) uracil
  misc_feature (26)..(28): n is 5-(N-3-phenylpropylcarboxyamide) uracil
  misc_feature (30)..(30): n is 5-(N-3-phenylpropylcarboxyamide) uracil
  misc_feature (32)..(32): n is 5-(N-3-phenylpropylcarboxyamide) uracil
  misc_feature (34)..(34): n is 5-(N-3-phenylpropylcarboxyamide) uracil
  misc_feature (36)..(36): n is 5-(N-3-phenylpropylcarboxyamide) uracil
nngncnangnagngngngngnggncnnngnangngnacag

SEQ ID NO: 79 - DNA, 39 bp, Artificial Sequence
  Synthetic
  misc_feature (8)..(8): n is 5-(N-3-phenylpropylcarboxyamide) uracil
  misc_feature (14)..(14): n is 5-(N-3-phenylpropylcarboxyamide) uracil
  misc_feature (16)..(16): n is 5-(N-3-phenylpropylcarboxyamide) uracil
  misc_feature (18)..(18): n is 5-(N-3-phenylpropylcarboxyamide) uracil
  misc_feature (20)..(22): n is 5-(N-3-phenylpropylcarboxyamide) uracil
  misc_feature (24)..(25): n is 5-(N-3-phenylpropylcarboxyamide) uracil
  misc_feature (27)..(27): n is 5-(N-3-phenylpropylcarboxyamide) uracil
  misc_feature (29)..(30): n is 5-(N-3-phenylpropylcarboxyamide) uracil
  misc_feature (32)..(32): n is 5-(N-3-phenylpropylcarboxyamide) uracil
  misc_feature (37)..(37): n is 5-(N-3-phenylpropylcarboxyamide) uracil
cgaggcanaccgangngnannngnncngnngnggacnga

SEQ ID NO: 80 - DNA, 40 bp, Artificial Sequence
  Synthetic
  misc_feature (1)..(1): n is 5-(N-3-phenylpropylcarboxyamide) uracil
  misc_feature (3)..(5): n is 5-(N-3-phenylpropylcarboxyamide) uracil
  misc_feature (7)..(7): n is 5-(N-3-phenylpropylcarboxyamide) uracil
  misc_feature (9)..(9): n is 5-(N-3-phenylpropylcarboxyamide) uracil
  misc_feature (11)..(11): n is 5-(N-3-phenylpropylcarboxyamide) uracil
  misc_feature (13)..(13): n is 5-(N-3-phenylpropylcarboxyamide) uracil
  misc_feature (16)..(17): n is 5-(N-3-phenylpropylcarboxyamide) uracil
  misc_feature (19)..(19): n is 5-(N-3-phenylpropylcarboxyamide) uracil
  misc_feature (21)..(22): n is 5-(N-3-phenylpropylcarboxyamide) uracil
  misc_feature (26)..(26): n is 5-(N-3-phenylpropylcarboxyamide) uracil
  misc_feature (31)..(32): n is 5-(N-3-phenylpropylcarboxyamide) uracil
  misc_feature (34)..(36): n is 5-(N-3-phenylpropylcarboxyamide) uracil
  misc_feature (39)..(39): n is 5-(N-3-phenylpropylcarboxyamide) uracil
nannncncngngncanngngnncgcncggcnngnnnganc

SEQ ID NO: 81 - DNA, 39 bp, Artificial Sequence
  Synthetic
  misc_feature (1)..(2): n is 5-(N-3-phenylpropylcarboxyamide) uracil
  misc_feature (4)..(4): n is 5-(N-3-phenylpropylcarboxyamide) uracil
  misc_feature (7)..(7): n is 5-(N-3-phenylpropylcarboxyamide) uracil
  misc_feature (9)..(9): n is 5-(N-3-phenylpropylcarboxyamide) uracil
  misc_feature (12)..(12): n is 5-(N-3-phenylpropylcarboxyamide) uracil
  misc_feature (14)..(14): n is 5-(N-3-phenylpropylcarboxyamide) uracil
  misc_feature (16)..(16): n is 5-(N-3-phenylpropylcarboxyamide) uracil
  misc_feature (18)..(19): n is 5-(N-3-phenylpropylcarboxyamide) uracil
  misc_feature (23)..(24): n is 5-(N-3-phenylpropylcarboxyamide) uracil
  misc_feature (28)..(29): n is 5-(N-3-phenylpropylcarboxyamide) uracil
  misc_feature (31)..(31): n is 5-(N-3-phenylpropylcarboxyamide) uracil
  misc_feature (33)..(35): n is 5-(N-3-phenylpropylcarboxyamide) uracil
  misc_feature (38)..(38): n is 5-(N-3-phenylpropylcarboxyamide) uracil
nngnccncngangngncnngacnncacnngngnnngcna

SEQ ID NO: 82 - DNA, 40 bp, Artificial Sequence
  Synthetic
  misc_feature (3)..(5): n is 5-(N-3-phenylpropylcarboxyamide) uracil
  misc_feature (7)..(7): n is 5-(N-3-phenylpropylcarboxyamide) uracil
  misc_feature (9)..(10): n is 5-(N-3-phenylpropylcarboxyamide) uracil
  misc_feature (12)..(13): n is 5-(N-3-phenylpropylcarboxyamide) uracil
  misc_feature (19)..(20): n is 5-(N-3-phenylpropylcarboxyamide) uracil
  misc_feature (22)..(22): n is 5-(N-3-phenylpropylcarboxyamide) uracil
  misc_feature (24)..(25): n is 5-(N-3-phenylpropylcarboxyamide) uracil
  misc_feature (29)..(31): n is 5-(N-3-phenylpropylcarboxyamide) uracil
  misc_feature (33)..(33): n is 5-(N-3-phenylpropylcarboxyamide) uracil
  misc_feature (35)..(35): n is 5-(N-3-phenylpropylcarboxyamide) uracil
  misc_feature (38)..(40): n is 5-(N-3-phenylpropylcarboxyamide) uracil
acnnngngnncnncacccnngncnnggcnnngncnccnnn

SEQ ID NO: 83 - DNA, 39 bp, Artificial Sequence
  Synthetic
  misc_feature (1)..(1): n is 5-(N-3-phenylpropylcarboxyamide) uracil
  misc_feature (3)..(3): n is 5-(N-3-phenylpropylcarboxyamide) uracil
  misc_feature (6)..(10): n is 5-(N-3-phenylpropylcarboxyamide) uracil
  misc_feature (12)..(13): n is 5-(N-3-phenylpropylcarboxyamide) uracil
  misc_feature (15)..(15): n is 5-(N-3-phenylpropylcarboxyamide) uracil
  misc_feature (17)..(17): n is 5-(N-3-phenylpropylcarboxyamide) uracil
  misc_feature (19)..(19): n is 5-(N-3-phenylpropylcarboxyamide) uracil
  misc_feature (22)..(22): n is 5-(N-3-phenylpropylcarboxyamide) uracil
  misc_feature (25)..(25): n is 5-(N-3-phenylpropylcarboxyamide) uracil
  misc_feature (30)..(31): n is 5-(N-3-phenylpropylcarboxyamide) uracil
  misc_feature (33)..(33): n is 5-(N-3-phenylpropylcarboxyamide) uracil
  misc_feature (35)..(35): n is 5-(N-3-phenylpropylcarboxyamide) uracil
  misc_feature (37)..(37): n is 5-(N-3-phenylpropylcarboxyamide) uracil
  misc_feature (39)..(39): n is 5-(N-3-phenylpropylcarboxyamide) uracil
nangcnnnnngnncngncnccncgncgacnncngncncn

SEQ ID NO: 84 - DNA, 39 bp, Artificial Sequence
  Synthetic
  misc_feature (1)..(3): n is 5-(N-3-phenylpropylcarboxyamide) uracil
  misc_feature (6)..(6): n is 5-(N-3-phenylpropylcarboxyamide) uracil
  misc_feature (8)..(8): n is 5-(N-3-phenylpropylcarboxyamide) uracil
  misc_feature (10)..(11): n is 5-(N-3-phenylpropylcarboxyamide) uracil
  misc_feature (13)..(14): n is 5-(N-3-phenylpropylcarboxyamide) uracil
  misc_feature (16)..(17): n is 5-(N-3-phenylpropylcarboxyamide) uracil
  misc_feature (19)..(20): n is 5-(N-3-phenylpropylcarboxyamide) uracil
  misc_feature (25)..(26): n is 5-(N-3-phenylpropylcarboxyamide) uracil
  misc_feature (28)..(28): n is 5-(N-3-phenylpropylcarboxyamide) uracil
  misc_feature (31)..(31): n is 5-(N-3-phenylpropylcarboxyamide) uracil
  misc_feature (33)..(33): n is 5-(N-3-phenylpropylcarboxyamide) uracil
  misc_feature (35)..(35): n is 5-(N-3-phenylpropylcarboxyamide) uracil
  misc_feature (39)..(39): n is 5-(N-3-phenylpropylcarboxyamide) uracil
nnncgngngnngnngnngnnacganngncgnangncggn

SEQ ID NO: 85 - DNA, 40 bp, Artificial Sequence
  Synthetic
  misc_feature (5)..(5): n is 5-(N-3-phenylpropylcarboxyamide) uracil
  misc_feature (10)..(11): n is 5-(N-3-phenylpropylcarboxyamide) uracil
  misc_feature (20)..(20): n is 5-(N-3-phenylpropylcarboxyamide) uracil
  misc_feature (22)..(24): n is 5-(N-3-phenylpropylcarboxyamide) uracil
  misc_feature (27)..(30): n is 5-(N-3-phenylpropylcarboxyamide) uracil
  misc_feature (32)..(32): n is 5-(N-3-phenylpropylcarboxyamide) uracil
  misc_feature (34)..(34): n is 5-(N-3-phenylpropylcarboxyamide) uracil
  misc_feature (36)..(37): n is 5-(N-3-phenylpropylcarboxyamide) uracil
agcgnagggnnaacggaacngnnnggnnnnangngnncac

SEQ ID NO: 86 - DNA, 40 bp, Artificial Sequence
  Synthetic
  misc_feature (1)..(3): n is 5-(N-3-phenylpropylcarboxyamide) uracil
  misc_feature (7)..(7): n is 5-(N-3-phenylpropylcarboxyamide) uracil
  misc_feature (10)..(10): n is 5-(N-3-phenylpropylcarboxyamide) uracil
  misc_feature (12)..(13): n is 5-(N-3-phenylpropylcarboxyamide) uracil
  misc_feature (16)..(16): n is 5-(N-3-phenylpropylcarboxyamide) uracil
  misc_feature (19)..(20): n is 5-(N-3-phenylpropylcarboxyamide) uracil
  misc_feature (22)..(22): n is 5-(N-3-phenylpropylcarboxyamide) uracil
  misc_feature (24)..(25): n is 5-(N-3-phenylpropylcarboxyamide) uracil
  misc_feature (27)..(27): n is 5-(N-3-phenylpropylcarboxyamide) uracil
  misc_feature (29)..(31): n is 5-(N-3-phenylpropylcarboxyamide) uracil
  misc_feature (35)..(36): n is 5-(N-3-phenylpropylcarboxyamide) uracil
  misc_feature (39)..(40): n is 5-(N-3-phenylpropylcarboxyamide) uracil
nnnggancangnnacnccnnangnngngnnnagcnngcnn

SEQ ID NO: 87 - DNA, 40 bp, Artificial Sequence
  Synthetic
  misc_feature (2)..(5): n is 5-(N-3-phenylpropylcarboxyamide) uracil
  misc_feature (7)..(7): n is 5-(N-3-phenylpropylcarboxyamide) uracil
  misc_feature (9)..(9): n is 5-(N-3-phenylpropylcarboxyamide) uracil
  misc_feature (16)..(17): n is 5-(N-3-phenylpropylcarboxyamide) uracil
  misc_feature (20)..(20): n is 5-(N-3-phenylpropylcarboxyamide) uracil
  misc_feature (22)..(22): n is 5-(N-3-phenylpropylcarboxyamide) uracil
  misc_feature (24)..(25): n is 5-(N-3-phenylpropylcarboxyamide) uracil
  misc_feature (27)..(27): n is 5-(N-3-phenylpropylcarboxyamide) uracil
  misc_feature (29)..(30): n is 5-(N-3-phenylpropylcarboxyamide) uracil
  misc_feature (33)..(33): n is 5-(N-3-phenylpropylcarboxyamide) uracil
  misc_feature (36)..(36): n is 5-(N-3-phenylpropylcarboxyamide) uracil
  misc_feature (38)..(38): n is 5-(N-3-phenylpropylcarboxyamide) uracil
  misc_feature (40)..(40): n is 5-(N-3-phenylpropylcarboxyamide) uracil
cnnnngnanagggganncgnangnngngnnccngangnan

SEQ ID NO: 88 - DNA, 40 bp, Artificial Sequence
  Synthetic
  misc_feature (10)..(10): n is 5-(N-3-phenylpropylcarboxyamide) uracil
  misc_feature (12)..(12): n is 5-(N-3-phenylpropylcarboxyamide) uracil
  misc_feature (14)..(14): n is 5-(N-3-phenylpropylcarboxyamide) uracil
  misc_feature (17)..(18): n is 5-(N-3-phenylpropylcarboxyamide) uracil
  misc_feature (21)..(21): n is 5-(N-3-phenylpropylcarboxyamide) uracil
  misc_feature (23)..(23): n is 5-(N-3-phenylpropylcarboxyamide) uracil
  misc_feature (25)..(26): n is 5-(N-3-phenylpropylcarboxyamide) uracil
  misc_feature (28)..(28): n is 5-(N-3-phenylpropylcarboxyamide) uracil
  misc_feature (36)..(39): n is 5-(N-3-phenylpropylcarboxyamide) uracil
cgggaacacngncngannaangncnngnaagcgggnnnnc

SEQ ID NO: 89 - DNA, 35 bp, Artificial Sequence
  Synthetic
  misc_feature (1)..(3): n is 5-(N-3-phenylpropylcarboxyamide) uracil
  misc_feature (7)..(8): n is 5-(N-3-phenylpropylcarboxyamide) uracil
  misc_feature (10)..(10): n is 5-(N-3-phenylpropylcarboxyamide) uracil
  misc_feature (13)..(14): n is 5-(N-3-phenylpropylcarboxyamide) uracil
  misc_feature (16)..(17): n is 5-(N-3-phenylpropylcarboxyamide) uracil
  misc_feature (19)..(19): n is 5-(N-3-phenylpropylcarboxyamide) uracil
  misc_feature (21)..(22): n is 5-(N-3-phenylpropylcarboxyamide) uracil
  misc_feature (26)..(27): n is 5-(N-3-phenylpropylcarboxyamide) uracil
  misc_feature (29)..(29): n is 5-(N-3-phenylpropylcarboxyamide) uracil
  misc_feature (32)..(32): n is 5-(N-3-phenylpropylcarboxyamide) uracil
nnnagcnngnganngnngnannacgnngnggncca

SEQ ID NO: 90 - DNA, 42 bp, Artificial Sequence
  Synthetic
  misc_feature (2)..(3): n is 5-(N-3-phenylpropylcarboxyamide) uracil
  misc_feature (5)..(5): n is 5-(N-3-phenylpropylcarboxyamide) uracil
  misc_feature (8)..(9): n is 5-(N-3-phenylpropylcarboxyamide) uracil
  misc_feature (11)..(12): n is 5-(N-3-phenylpropylcarboxyamide) uracil
  misc_feature (14)..(15): n is 5-(N-3-phenylpropylcarboxyamide) uracil
  misc_feature (17)..(17): n is 5-(N-3-phenylpropylcarboxyamide) uracil
  misc_feature (21)..(22): n is 5-(N-3-phenylpropylcarboxyamide) uracil
  misc_feature (24)..(25): n is 5-(N-3-phenylpropylcarboxyamide) uracil
  misc_feature (28)..(28): n is 5-(N-3-phenylpropylcarboxyamide) uracil
  misc_feature (32)..(34): n is 5-(N-3-phenylpropylcarboxyamide) uracil
  misc_feature (36)..(37): n is 5-(N-3-phenylpropylcarboxyamide) uracil
  misc_feature (41)..(41): n is 5-(N-3-phenylpropylcarboxyamide) uracil
cnncnccnngnngnncnccanngnncgncgcnnngnnaccnc

SEQ ID NO: 91 - DNA, 40 bp, Artificial Sequence
  Synthetic
  misc_feature (1)..(5): n is 5-(N-3-phenylpropylcarboxyamide) uracil
  misc_feature (7)..(7): n is 5-(N-3-phenylpropylcarboxyamide) uracil
  misc_feature (10)..(10): n is 5-(N-3-phenylpropylcarboxyamide) uracil
  misc_feature (14)..(14): n is 5-(N-3-phenylpropylcarboxyamide) uracil
  misc_feature (17)..(18): n is 5-(N-3-phenylpropylcarboxyamide) uracil
  misc_feature (22)..(22): n is 5-(N-3-phenylpropylcarboxyamide) uracil
  misc_feature (24)..(24): n is 5-(N-3-phenylpropylcarboxyamide) uracil
  misc_feature (26)..(26): n is 5-(N-3-phenylpropylcarboxyamide) uracil
  misc_feature (28)..(28): n is 5-(N-3-phenylpropylcarboxyamide) uracil
  misc_feature (31)..(31): n is 5-(N-3-phenylpropylcarboxyamide) uracil
  misc_feature (33)..(33): n is 5-(N-3-phenylpropylcarboxyamide) uracil
  misc_feature (35)..(35): n is 5-(N-3-phenylpropylcarboxyamide) uracil
  misc_feature (40)..(40): n is 5-(N-3-phenylpropylcarboxyamide) uracil
nnnnnanccngcgnacnnccgncngngnccnancnccacn

SEQ ID NO: 92 - DNA, 40 bp, Artificial Sequence
  Synthetic
  misc_feature (1)..(2): n is 5-(N-3-phenylpropylcarboxyamide) uracil
  misc_feature (5)..(6): n is 5-(N-3-phenylpropylcarboxyamide) uracil
  misc_feature (13)..(15): n is 5-(N-3-phenylpropylcarboxyamide) uracil
  misc_feature (18)..(18): n is 5-(N-3-phenylpropylcarboxyamide) uracil
  misc_feature (20)..(20): n is 5-(N-3-phenylpropylcarboxyamide) uracil
  misc_feature (22)..(22): n is 5-(N-3-phenylpropylcarboxyamide) uracil
  misc_feature (25)..(26): n is 5-(N-3-phenylpropylcarboxyamide) uracil
  misc_feature (28)..(28): n is 5-(N-3-phenylpropylcarboxyamide) uracil
  misc_feature (32)..(33): n is 5-(N-3-phenylpropylcarboxyamide) uracil
  misc_feature (35)..(36): n is 5-(N-3-phenylpropylcarboxyamide) uracil
  misc_feature (39)..(40): n is 5-(N-3-phenylpropylcarboxyamide) uracil
nngannccgaccnnngangngncgnncnggcnnannccnn

SEQ ID NO: 93 - DNA, 40 bp, Artificial Sequence
  Synthetic
  misc_feature (1)..(1): n is 5-(N-3-phenylpropylcarboxyamide) uracil
  misc_feature (3)..(3): n is 5-(N-3-phenylpropylcarboxyamide) uracil
  misc_feature (10)..(15): n is 5-(N-3-phenylpropylcarboxyamide) uracil
  misc_feature (17)..(17): n is 5-(N-3-phenylpropylcarboxyamide) uracil
  misc_feature (19)..(19): n is 5-(N-3-phenylpropylcarboxyamide) uracil
  misc_feature (22)..(22): n is 5-(N-3-phenylpropylcarboxyamide) uracil
  misc_feature (24)..(24): n is 5-(N-3-phenylpropylcarboxyamide) uracil
  misc_feature (26)..(26): n is 5-(N-3-phenylpropylcarboxyamide) uracil
  misc_feature (28)..(28): n is 5-(N-3-phenylpropylcarboxyamide) uracil
  misc_feature (33)..(33): n is 5-(N-3-phenylpropylcarboxyamide) uracil
  misc_feature (39)..(40): n is 5-(N-3-phenylpropylcarboxyamide) uracil
nanccgcccnnnnnnancnccngngngnccgcngcaaann

SEQ ID NO: 94 - DNA, 39 bp, Artificial Sequence
  Synthetic
  misc_feature (1)..(2): n is 5-(N-3-phenylpropylcarboxyamide) uracil
  misc_feature (4)..(4): n is 5-(N-3-phenylpropylcarboxyamide) uracil
  misc_feature (7)..(7): n is 5-(N-3-phenylpropylcarboxyamide) uracil
  misc_feature (9)..(9): n is 5-(N-3-phenylpropylcarboxyamide) uracil
  misc_feature (11)..(14): n is 5-(N-3-phenylpropylcarboxyamide) uracil
  misc_feature (16)..(16): n is 5-(N-3-phenylpropylcarboxyamide) uracil
  misc_feature (20)..(21): n is 5-(N-3-phenylpropylcarboxyamide) uracil
  misc_feature (24)..(24): n is 5-(N-3-phenylpropylcarboxyamide) uracil
  misc_feature (26)..(27): n is 5-(N-3-phenylpropylcarboxyamide) uracil
  misc_feature (29)..(29): n is 5-(N-3-phenylpropylcarboxyamide) uracil
  misc_feature (32)..(32): n is 5-(N-3-phenylpropylcarboxyamide) uracil
  misc_feature (34)..(35): n is 5-(N-3-phenylpropylcarboxyamide) uracil
nncngcncnannnngncccnngcnanncngcncnnaccc

SEQ ID NO: 95 - DNA, 39 bp, Artificial Sequence
  Synthetic
  misc_feature (7)..(7): n is 5-(N-3-phenylpropylcarboxyamide) uracil
  misc_feature (10)..(11): n is 5-(N-3-phenylpropylcarboxyamide) uracil
  misc_feature (13)..(13): n is 5-(N-3-phenylpropylcarboxyamide) uracil
  misc_feature (15)..(15): n is 5-(N-3-phenylpropylcarboxyamide) uracil
  misc_feature (18)..(20): n is 5-(N-3-phenylpropylcarboxyamide) uracil
  misc_feature (23)..(23): n is 5-(N-3-phenylpropylcarboxyamide) uracil
  misc_feature (27)..(27): n is 5-(N-3-phenylpropylcarboxyamide) uracil
  misc_feature (30)..(30): n is 5-(N-3-phenylpropylcarboxyamide) uracil
  misc_feature (32)..(32): n is 5-(N-3-phenylpropylcarboxyamide) uracil
  misc_feature (38)..(39): n is 5-(N-3-phenylpropylcarboxyamide) uracil
aacaccngcnngncncgnnncgncaanggnancggccnn

SEQ ID NO: 96 - DNA, 41 bp, Artificial Sequence
  Synthetic
  misc_feature (1)..(1): n is 5-(N-3-phenylpropylcarboxyamide) uracil
  misc_feature (3)..(3): n is 5-(N-3-phenylpropylcarboxyamide) uracil
  misc_feature (5)..(5): n is 5-(N-3-phenylpropylcarboxyamide) uracil
  misc_feature (7)..(7): n is 5-(N-3-phenylpropylcarboxyamide) uracil
  misc_feature (10)..(11): n is 5-(N-3-phenylpropylcarboxyamide) uracil
  misc_feature (13)..(14): n is 5-(N-3-phenylpropylcarboxyamide) uracil
  misc_feature (17)..(18): n is 5-(N-3-phenylpropylcarboxyamide) uracil
  misc_feature (21)..(21): n is 5-(N-3-phenylpropylcarboxyamide) uracil
  misc_feature (24)..(24): n is 5-(N-3-phenylpropylcarboxyamide) uracil
  misc_feature (26)..(27): n is 5-(N-3-phenylpropylcarboxyamide) uracil
  misc_feature (30)..(30): n is 5-(N-3-phenylpropylcarboxyamide) uracil
  misc_feature (33)..(33): n is 5-(N-3-phenylpropylcarboxyamide) uracil
  misc_feature (35)..(35): n is 5-(N-3-phenylpropylcarboxyamide) uracil
  misc_feature (39)..(40): n is 5-(N-3-phenylpropylcarboxyamide) uracil
nanangncgnngnnacnnccnccncnnacngancnacanna

SEQ ID NO: 97 - DNA, 40 bp, Artificial Sequence
  Synthetic
  misc_feature (1)..(3): n is 5-(N-3-phenylpropylcarboxyamide) uracil
  misc_feature (6)..(6): n is 5-(N-3-phenylpropylcarboxyamide) uracil
  misc_feature (8)..(8): n is 5-(N-3-phenylpropylcarboxyamide) uracil
  misc_feature (13)..(15): n is 5-(N-3-phenylpropylcarboxyamide) uracil
  misc_feature (17)..(17): n is 5-(N-3-phenylpropylcarboxyamide) uracil
  misc_feature (19)..(19): n is 5-(N-3-phenylpropylcarboxyamide) uracil
  misc_feature (21)..(21): n is 5-(N-3-phenylpropylcarboxyamide) uracil
  misc_feature (23)..(23): n is 5-(N-3-phenylpropylcarboxyamide) uracil
  misc_feature (25)..(26): n is 5-(N-3-phenylpropylcarboxyamide) uracil
  misc_feature (30)..(31): n is 5-(N-3-phenylpropylcarboxyamide) uracil
  misc_feature (34)..(34): n is 5-(N-3-phenylpropylcarboxyamide) uracil
  misc_feature (37)..(37): n is 5-(N-3-phenylpropylcarboxyamide) uracil
  misc_feature (39)..(40): n is 5-(N-3-phenylpropylcarboxyamide) uracil
nnnggnanaagcnnnangngngncnnaggnnccncancnn

SEQ ID NO: 98 - DNA, 40 bp, Artificial Sequence
  Synthetic
  misc_feature (2)..(2): n is 5-(N-3-phenylpropylcarboxyamide) uracil
  misc_feature (8)..(12): n is 5-(N-3-phenylpropylcarboxyamide) uracil
  misc_feature (14)..(14): n is 5-(N-3-phenylpropylcarboxyamide) uracil
  misc_feature (16)..(16): n is 5-(N-3-phenylpropylcarboxyamide) uracil
  misc_feature (20)..(22): n is 5-(N-3-phenylpropylcarboxyamide) uracil
  misc_feature (26)..(26): n is 5-(N-3-phenylpropylcarboxyamide) uracil
  misc_feature (28)..(29): n is 5-(N-3-phenylpropylcarboxyamide) uracil
  misc_feature (31)..(31): n is 5-(N-3-phenylpropylcarboxyamide) uracil
  misc_feature (33)..(33): n is 5-(N-3-phenylpropylcarboxyamide) uracil
  misc_feature (35)..(35): n is 5-(N-3-phenylpropylcarboxyamide) uracil
  misc_feature (38)..(39): n is 5-(N-3-phenylpropylcarboxyamide) uracil
gnccaacnnnnncngncagnnngggncnngngngncgnng

SEQ ID NO: 99 - DNA, 40 bp, Artificial Sequence
  Synthetic
  misc_feature (1)..(2): n is 5-(N-3-phenylpropylcarboxyamide) uracil
  misc_feature (4)..(4): n is 5-(N-3-phenylpropylcarboxyamide) uracil
  misc_feature (6)..(7): n is 5-(N-3-phenylpropylcarboxyamide) uracil
  misc_feature (9)..(9): n is 5-(N-3-phenylpropylcarboxyamide) uracil
  misc_feature (11)..(11): n is 5-(N-3-phenylpropylcarboxyamide) uracil
  misc_feature (13)..(13): n is 5-(N-3-phenylpropylcarboxyamide) uracil
  misc_feature (15)..(15): n is 5-(N-3-phenylpropylcarboxyamide) uracil
  misc_feature (18)..(18): n is 5-(N-3-phenylpropylcarboxyamide) uracil
  misc_feature (21)..(22): n is 5-(N-3-phenylpropylcarboxyamide) uracil
  misc_feature (25)..(25): n is 5-(N-3-phenylpropylcarboxyamide) uracil
  misc_feature (28)..(28): n is 5-(N-3-phenylpropylcarboxyamide) uracil
  misc_feature (34)..(36): n is 5-(N-3-phenylpropylcarboxyamide) uracil
  misc_feature (38)..(39): n is 5-(N-3-phenylpropylcarboxyamide) uracil
nngncnngngngncnggnccnnccncancggccnnncnna

SEQ ID NO: 100 - DNA, 24 bp, Artificial Sequence
  Synthetic
  misc_feature (1)..(1): n is 5-(N-3-phenylpropylcarboxyamide) uracil
  misc_feature (3)..(3): n is 5-(N-3-phenylpropylcarboxyamide) uracil
  misc_feature (5)..(5): n is 5-(N-3-phenylpropylcarboxyamide) uracil
  misc_feature (7)..(7): n is a, g, c, or t
  misc_feature (8)..(8): n is 5-(N-3-phenylpropylcarboxyamide) uracil
  misc_feature (10)..(10): n is 5-(N-3-phenylpropylcarboxyamide) uracil
  misc_feature (12)..(13): n is 5-(N-3-phenylpropylcarboxyamide) uracil
  misc_feature (14)..(14): n is a, g, c, or t
  misc_feature (17)..(17): n is 5-(N-3-phenylpropylcarboxyamide) uracil
  misc_feature (20)..(20): n is 5-(N-3-phenylpropylcarboxyamide) uracil
ngngncnngngnnnaangangagg

(19) United States
(12) Patent Application Publication
(10) Pub. No.: US 2022/0333113 A1
(43) Pub. Date: Oct. 20, 2022

(54) Nucleic Acid Compounds that Bind to Retinoic Acid-Inducible Gene I Protein

(71) Applicant: Somalogic Operating Co., Inc., Boulder, CO (US)

(72) Inventors: Nebojsa Janjic, Boulder, CO (US); Amy Gelinas, Boulder, CO (US)

(73) Assignee: Somalogic Operating Co., Inc., Boulder, CO (US)

(21) Appl. No.: 17/766,013

(22) PCT Filed: Oct. 15, 2020
(86) PCT No.: PCT/US2020/055711

§ 371 Date: Apr. 1, 2022

Related U.S. Application Data
(60) Provisional application No. 62/915,842, filed on Oct. 16, 2019.

(51) Int. Cl. C12N 15/115

(52) U.S. Cl. CPC C12N 15/115; C12N 2310/16; C12N 2320/30

(57) ABSTRACT

Described herein are aptamers capable of binding to human retinoic acid-inducible gene I protein (RIG-I); compositions comprising a RIG-I binding aptamer with a RIG-I; and methods of making and using the same.

### CROSS-REFERENCE TO RELATED APPLICATIONS

This application claims the benefit of priority of U.S. Provisional Application No. 62/915,842, filed Oct. 16, 2019, which is incorporated by reference herein in its entirety for any purpose.

### FIELD

The present disclosure relates generally to the field of nucleic acids, and more specifically, to aptamers capable of binding to human retinoic acid-inducible gene I protein (RIG-I), compositions comprising a RIG-I binding aptamer and RIG-I, and methods of detecting RIG-I using such aptamers.

### BACKGROUND

RIG-I is a receptor that plays a role in innate antiviral immunity by binding viral RNAs and initiating signaling for interferon (IFN) production. The C-terminal domain (CTD) of RIG-I is the critical motif for detecting the viral RNAs and binds with high affinity to tri- and diphosphate moieties on the 5′ terminus of blunt-ended RNA duplexes that are found in the folded “panhandle” structures of many viral genomes or on viral replication intermediates. Mechanistically, RNA binding to the RIG-I CTD induces conformational changes in the overall RIG-I protein structure that initiates a signaling cascade resulting in the initiation of IFN induction.

Though RIG-I is an essential receptor for antiviral immunity, hyperactivation of the receptor is linked to a variety of pathologies from autoimmunity to chronic obstructive pulmonary disease (COPD). Given the critical roles played by the CTD, and the link between RIG-I and several different pathologies, a protein binding aptamer that binds specifically to the RIG-I protein, e.g, the CTD of RIG-I, would be beneficial.

The present disclosure provides aptamers that specifically bind to human RIG-I protein.

### SUMMARY

The present disclosure describes aptamers capable of binding to human retinoic acid-inducible gene I protein (RIG-I). Methods of making and using the same are described.

In some embodiments, an aptamer that binds RIG-I protein is provided. In some embodiments, an aptamer that binds RIG-I protein comprises the sequence 5′- PEPSZV -3′ (SEQ ID NO: 49), wherein each P is independently, and for each occurrence, a C-5 modified pyrimidine; E is a C-5 modified pyrimidine, A or G; S is a G or C; Z is a C-5 modified pyrimidine or A; and V is a C, A, or G. In some embodiments, an aptamer that binds RIG-I protein comprises the sequence 5′-PEPSFP-3′ (SEQ ID NO: 50), wherein each P is independently, and for each occurrence, a C-5 modified pyrimidine; E is a C-5 modified pyrimidine, A or G; S is a G or C; and F is a C-5 modified pyrimidine, unmodified C, G, or A. In some such embodiments, the aptamer comprises SEQ ID NOs: 49 and 50. In some such embodiments, the aptamer comprises SEQ ID NOs: 49 and 50 and the sequence 5′-AAPGAPGAGG-3′ (SEQ ID NO: 51). In some such embodiments, the aptamer is at least 30, 40, 50, 60, 70, 80, 90, or 100 nucleotides in length.

In some embodiments, an aptamer that binds RIG-I protein comprises the sequence 5′-PGPGPCAₙPGPGPPPZAZQQCₙZMGPPAAPGAPGAGG -3′ (SEQ ID NO: 52), wherein P is independently, and for each occurrence, a C-5 modified pyrimidine; Z is independently, and for each occurrence, a C-5 modified pyrimidine or A; Q is independently, and for each occurrence, a C-5 modified pyrimidine or G; M is a C or A; and subscript n is independently, and for each occurrence, 0 or 1.

In some embodiments, each C-5 modified pyrimidine containing nucleoside is independently selected from 5-(N-benzylcarboxyamide)-2′-deoxyuridine (BndU), 5-(N-benzylcarboxyamide)-2′-O-methyluridine, 5-(N-benzylcarboxyamide)-2′-fluorouridine, 5-(N-phenethylcarboxyamide)-2′-deoxyuridine (PEdU), 5-(N-thiophenylmethylcarboxyamide)-2′-deoxyuridine (ThdU), 5-(N-isobutylcarboxyamide)-2′-deoxyuridine (iBudU), 5-(N-tyrosylcarboxyamide)-2′-deoxyuridine (TyrdU), 5-(N-3,4-methylenedioxybenzylcarboxyamide)-2′-deoxyuridine (MBndU), 5-(N-4-fluorobenzylcarboxyamide)-2′-deoxyuridine (FBndU), 5-(N-3-phenylpropylcarboxyamide)-2′-deoxyuridine (PPdU), 5-(N-imidizolylethylcarboxyamide)-2′-deoxyuridine (ImdU), 5-(N-isobutylcarboxyamide)-2′-O-methyluridine, 5-(N-isobutylcarboxyamide)-2′-fluorouridine, 5-(N-tryptaminocarboxyamide)-2′-deoxyuridine (TrpdU), 5-(N-R-threoninylcarboxyamide)-2′-deoxyuridine (ThrdU), 5-(N-tryptaminocarboxyamide)-2′-O-methyluridine, 5-(N-tryptaminocarboxyamide)-2′-fluorouridine, 5-(N-[1-(3-trimethylamonium) propyl]carboxyamide)-2′-deoxyuridine chloride, 5-(N-naphthylmethylcarboxyamide)-2′-deoxyuridine (NapdU), 5-(N-naphthylmethylcarboxyamide)-2′-O-methyluridine, 5-(N-naphthylmethylcarboxyamide)-2′-fluorouridine, 5-(N-[1-(2,3-dihydroxypropyl)]3 carboxyamide)-2′-deoxyuridine), 5-(N-2-naphthylmethylcarboxyamide)-2′-deoxyuridine (2NapdU), 5-(N-2-naphthylmethylcarboxyamide)-2′-O-methyluridine, 5-(N-2-naphthylmethylcarboxyamide)-2′-fluorouridine, 5-(N-1-naphthylethylcarboxyamide)-2′-deoxyuridine (NEdU), 5-(N-1-naphthylethylcarboxyamide)-2′-O-methyluridine, 5-(N-1-naphthylethylcarboxyamide)-2′-fluorouridine, 5-(N-2-naphthylethylcarboxyamide)-2′-deoxyuridine (2NEdU), 5-(N-2-naphthylethylcarboxyamide)-2′-O-methyluridine, 5-(N-2-naphthylethylcarboxyamide)-2′-fluorouridine, 5-(N-3-benzofuranylethylcarboxyamide)-2′-deoxyuridine (BFdU), 5-(N-3-benzofuranylethylcarboxyamide)-2′-O-methyluridine, 5-(N-3-benzofuranylethylcarboxyamide)-2′-fluorouridine, 5-(N-3-benzothiophenylethylcarboxyamide)-2′-deoxyuridine (BTdU), 5-(N-3-benzothiophenylethylcarboxyamide)-2′-O-methyluridine, and 5-(N-3-benzothiophenylethylcarboxyamide)-2′-fluorouridine.

In some embodiments, each C-5 modified pyrimidine containing nucleoside is 5-(N-3-phenylpropylcarboxyamide)-2′-deoxyuridine (PPdU).

In some embodiments, the aptamer comprises one or more sequences selected from SEQ ID NOs : 65-67.

In some embodiments, an aptamer that binds RIG-I protein comprises a sequence that is at least 80%, at least 85%, at least 90%, at least 95%, at least 97%, at least 99%, or 100% identical to a sequence selected from SEQ ID NOs: 4-47 and 68-99 .

In some embodiments, the aptamer is from 5 to 60 nucleotides in length, or from 35 to 50 nucleotides in length, or from 40 to 50 nucleotides in length.

In some embodiments, the RIG-I protein that the aptamer binds is a human RIG-I protein.

In some embodiments, methods for selecting an aptamer having binding affinity for a RIG-I protein are provided. In some embodiments, such a method comprises selecting an aptamer having binding affinity for a RIG-I protein comprising: contacting a candidate mixture with a RIG-I protein, wherein the candidate mixture comprises modified nucleic acids in which one, several or all pyrimidines in at least one, or each, nucleic acid of the candidate mixture comprises a C-5 modified pyrimidine; exposing the candidate mixture to a slow off-rate enrichment process, wherein nucleic acids having a slow rate of dissociation from the target molecule relative to other nucleic acids in the candidate mixture bind the RIG-I protein, forming nucleic acid-target molecule complexes; partitioning slow off-rate nucleic acids from the candidate mixture; amplifying the slow off-rate nucleic acids to yield a mixture of nucleic acids enriched in nucleic acid sequences that are capable of binding to the RIG-I protein with a slow off-rate, whereby a slow off-rate aptamer to the RIG-I protein molecule is selected.

In some embodiments, the candidate mixture comprises nucleic acids comprising the sequence 5′- PEPSZV -3′ (SEQ ID NO: 49), wherein each P is independently, and for each occurrence, a C-5 modified pyrimidine; E is a C-5 modified pyrimidine, A or G; S is a G or C; Z is a C-5 modified pyrimidine or A; and V is a C, A, or G. In some embodiments, the candidate mixture comprises nucleic acids comprising the sequence 5′-PEPSFP-3′ (SEQ ID NO: 50), wherein each P is independently, and for each occurrence, a C-5 modified pyrimidine; E is a C-5 modified pyrimidine, A or G; S is a G or C; and F is a C-5 modified pyrimidine, unmodified C, G, or A. In some embodiments, the candidate mixture comprises nucleic acids comprising the sequence 5′-AAPGAPGAGG-3′ (SEQ ID NO: 51).

In some embodiments, each nucleic acid is, independently, from 35 to 60 nucleotides in length, or from 35 to 50 nucleotides in length, or from 40 to 50 nucleotides in length.

In some embodiments, each C-5 modified pyrimidine containing nucleoside is independently selected from: 5-(N-benzylcarboxyamide)-2′-deoxyuridine (BndU), 5-(N-benzylcarboxyamide)-2′-O-methyluridine, 5-(N-benzylcarboxyamide)-2′-fluorouridine, 5-(N-phenethylcarboxyamide)-2′-deoxyuridine (PEdU), 5-(N-thiophenylmethylcarboxyamide)-2′-deoxyuridine (ThdU), 5-(N-isobutylcarboxyamide)-2′-deoxyuridine (iBudU), 5-(N-tyrosylcarboxyamide)-2′-deoxyuridine (TyrdU), 5-(N-3,4-methylenedioxybenzylcarboxyamide)-2′-deoxyuridine (MBndU), 5-(N-4-fluorobenzylcarboxyamide)-2′-deoxyuridine (FBndU), 5-(N-3-phenylpropylcarboxyamide)-2′-deoxyuridine (PPdU), 5-(N-imidizolylethylcarboxyamide)-2′-deoxyuridine (ImdU), 5-(N-isobutylcarboxyamide)-2′-O-methyluridine, 5-(N-isobutylcarboxyamide)-2′-fluorouridine, 5-(N-tryptaminocarboxyamide)-2′-deoxyuridine (TrpdU), 5-(N-R-threoninylcarboxyamide)-2′-deoxyuridine (ThrdU), 5-(N-tryptaminocarboxyamide)-2′-O-methyluridine, 5-(N-tryptaminocarboxyamide)-2′-fluorouridine, 5-(N-[1-(3-trimethylamonium) propyl] carboxyamide)-2′-deoxyuridine chloride, 5-(N-naphthylmethylcarboxyamide)-2′-deoxyuridine (NapdU), 5-(N-naphthylmethylcarboxyamide)-2′-O-methyluridine, 5-(N-naphthylmethylcarboxyamide)-2′-fluorouridine, 5-(N-[1-(2,3-dihydroxypropyl)]carboxyamide)-2′-deoxyuridine), 5-(N-2-naphthylmethylcarboxyamide)-2′-deoxyuridine (2NapdU), 5-(N-2-naphthylmethylcarboxyamide)-2′-O-methyluridine, 5-(N-2-naphthylmethylcarboxyamide)-2′-fluorouridine, 5-(N-1-naphthylethylcarboxyamide)-2′-deoxyuridine (NEdU), 5-(N-1-naphthylethylcarboxyamide)-2′-O-methyluridine, 5-(N-1-naphthylethylcarboxyamide)-2′-fluorouridine, 5-(N-2-naphthylethylcarboxyamide)-2′-deoxyuridine (2NEdU), 5-(N-2-naphthylethylcarboxyamide)-2′-O-methyluridine, 5-(N-2-naphthylethylcarboxyamide)-2′-fluorouridine, 5-(N-3-benzofuranylethylcarboxyamide)-2′-deoxyuridine (BFdU), 5-(N-3-benzofuranylethylcarboxyamide)-2′-O-methyluridine, 5-(N-3-benzofuranylethylcarboxyamide)-2′-fluorouridine, 5-(N-3-benzothiophenylethylcarboxyamide)-2′-deoxyuridine (BTdU), 5-(N-3-benzothiophenylethylcarboxyamide)-2′-O-methyluridine, and 5-(N-3-benzothiophenylethylcarboxyamide)-2′-fluorouridine.

In some embodiments, each C-5 modified pyrimidine containing nucleoside is 5-(N-3-phenylpropylcarboxyamide)-2′-deoxyuridine (PPdU).

In some embodiments, the RIG-I protein is a human RIG-I protein.

The foregoing and other objects, features, and advantages of the invention will become more apparent from the following detailed description, which proceeds with reference to the accompanying figures.

### BRIEF DESCRIPTION OF THE DRAWINGS

FIG. 1A shows an alignment of the 40 nucleotide randomized regions of thirty-two independently derived aptamer sequences. Four patterns identified from the thirty-two aptamer sequences are shown. “J” is 5-(N-3-phenylpropylcarboxyamide) uracil. FIG. 1B shows the 40 nucleotide randomized regions of twenty-six independently derived aptamer sequences. The nucleobases that align to Pattern 4 (SEQ ID NO: 65) are highlighted within each sequence. FIG. 1C shows the nucleotide positions and consensus sequence for Pattern 4 based upon the 26 independent sequences shown in FIG. 1B. The frequency of each nucleobase in aptamer Pattern 4 at each position that defines the consensus sequence is also shown. FIG. 1D shows the 40 nucleotide randomized regions of seventeen independently derived aptamer sequences. The nucleobases that align to Pattern 3 (SEQ ID NO: 66) are highlighted within each sequence. FIG. 1E shows the nucleotide positions and consensus sequence for Pattern 3 based upon the 17 independent sequences shown in FIG. 1D. The frequency of each nucleobase in aptamer Pattern 3 at each position that defines the consensus sequence is also shown. FIG. 1F shows the 40 nucleotide randomized regions of two independently derived aptamer sequences. The nucleobases that align to a portion of Pattern 1 (SEQ ID NO: 67) are highlighted within each sequence. FIG. 1G shows the nucleotide positions and consensus sequence for the portion of Pattern 1 based upon the two independent sequences shown in FIG. 1F. The frequency of each nucleobase in the portion of aptamer Pattern 1 at each position that defines the consensus sequence is also shown. FIG. 1H shows the nucleotide position and consensus sequence for combined Patterns 1 to 4 based upon the independently derived aptamer sequences. The frequency of each nucleobase at each position that defines the consensus sequence is also shown.

FIG. 2A shows a diagram of protein domains of full-length RIG-I protein and various RIG-I protein truncates. In FIG. 2A, “CARDs” means caspase activation and recruitment domains, “HD” means helicase domain, and “CTD” means C-terminal regulatory domain. The diagram shown in FIG. 2A is from Vela et al. The Thermodynamic Basis for Viral RNA Detection by the RIG-I Innate Immune Sensor. J. Biol. Chem. 287(51): 42564, 2012. FIG. 2B shows a graphical representation of the fraction of bound aptamers (y-axis) plotted as a function of protein concentration (x-axis). Human RIG-I-CTD protein concentration ranged from 1×10⁻⁸ M to 1×1⁻¹⁴ M, and the equilibrium binding constants (K_{d}) was calculated using y=(maximum.−minimum)(Pₜ)/(K_{d}+Pₜ)+minimum.

FIG. 3 shows certain exemplary 5-position modified uracils and cytosines that may be incorporated into aptamers.

FIG. 4 shows certain exemplary modifications that may be present at the 5-position of uracil. The chemical structure of the C-5 modification includes the exemplary amide linkage that links the modification to the 5-position of the uracil. The 5-position moieties shown include a benzyl moiety (e.g., Bn, PE and a PP), a naphthyl moiety (e.g., Nap, 2Nap, NE), a butyl moiety (e.g, iBu), a fluorobenzyl moiety (e.g., FBn), a tyrosyl moiety (e.g., a Tyr), a 3,4-methylenedioxy benzyl (e.g., MBn), a morpholino moiety (e.g., MOE), a benzofuranyl moiety (e.g., BF), an indole moiety (e.g, Trp) and a hydroxypropyl moiety (e.g., Thr).

FIG. 5 shows exemplary C-5 modified pyrimidine containing nucleosides and modifications that may be present at the 5-position of cytosine. The chemical structure of the C-5 modification includes the exemplary amide linkage that links the modification to the 5-position of the cytosine. The 5-position moieties shown include a benzyl moiety (e.g., Bn, PE and a PP), a naphthyl moiety (e.g., Nap, 2Nap, NE, and 2NE) and a tyrosyl moiety (e.g., a Tyr).

### DETAILED DESCRIPTION

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Aptamer: The term aptamer, as used herein, refers to a non-naturally occurring nucleic acid that has a desirable action on a target molecule. Desirable actions include, but are not limited to, binding of the target, enhancing the activity of the target, and inhibiting the activity of the target. An aptamer may also be referred to as a “nucleic acid ligand.” In some embodiments, an aptamer is a SOMAmer. As used herein, the term “aptamer” includes aptamers and pharmaceutically acceptable salts thereof, unless specifically indicated otherwise.

In some embodiments, an aptamer specifically binds RIG-I through a mechanism which is independent of Watson/Crick base pairing or triple helix formation, and wherein the aptamer does not have the known physiological function of being bound by RIG-I. In some embodiments, aptamers that bind RIG-I include nucleic acids that are identified from a candidate mixture of nucleic acids, by a method comprising: (a) contacting the candidate mixture with the target, wherein nucleic acids having an increased affinity to the target relative to other nucleic acids in the candidate mixture can be partitioned from the remainder of the candidate mixture; (b) partitioning the increased affinity nucleic acids from the remainder of the candidate mixture; and (c) amplifying the increased affinity nucleic acids to yield a ligand-enriched mixture of nucleic acids, whereby aptamers that bind RIG-I are identified. It is recognized that affinity interactions are a matter of degree; however, in this context, an aptamer that “specifically binds” its target means that the aptamer binds to its target with a much higher degree of affinity than it binds to other, non-target, components in a mixture or sample. An “aptamer” or “nucleic acid ligand” is a set of copies of one type or species of nucleic acid molecule that has a particular nucleotide sequence. An aptamer can include any suitable number of nucleotides. “Aptamers” refer to more than one such set of molecules. Different aptamers can have either the same or different numbers of nucleotides. Aptamers may comprise DNA, RNA, both DNA and RNA, and modified versions of either or both, and may be single stranded, double stranded, or contain double stranded or triple stranded regions, or any other three-dimensional structures.

Bioactivity: The term bioactivity, as used herein, refers to one or more intercellular, intracellular or extracellular process (e.g., cell-cell binding, ligand-receptor binding, cell signaling, etc.) which can impact physiological or pathophysiological processes.

C-5 Modified Pyrimidine: C-5 modified pyrimidine, as used herein, refers to a pyrimidine with a modification at the C-5 position. Examples of a C-5 modified pyrimidine include those described in U.S. Pat. Nos. 5,719,273 and 5,945,527. Certain nonlimiting examples of C-5 modified pyrimidines are provided herein.

RIG-I Aptamer: “RIG-I aptamer”, as used herein, refers to an aptamer that is capable of binding to a RIG-I protein.

Modified: As used herein, the terms “modify”, “modified”, “modification”, and any variations thereof, when used in reference to an oligonucleotide, means that the oligonucleotide comprises at least one non-natural moiety, such as at least one non-natural sugar moiety, at least one non-natural internucleoside linkage, at least one non-natural nucleotide base moiety, and/or at least one moiety that does not naturally occur in oligonucleotides (such as, for example, a 3 carbon spacer or a hexaethylene glycol (HEG)). In some embodiments, at least one of the four constituent nucleotide bases (i.e., A, G, T/U, and C) of the oligonucleotide is a modified nucleotide. In some such embodiments, the modified nucleotide comprises a base moiety that is more hydrophobic than the naturally-occurring base. In some embodiments, the modified nucleotide confers nuclease resistance to the oligonucleotide. In some embodiments, when an aptamer comprises one or more modified nucleotides that comprise hydrophobic base moieties, the aptamer binds to its target, such as a protein, through predominantly hydrophobic interactions. In some embodiments, such hydrophobic interactions result in high binding efficiency and stable co-crystal complexes. A pyrimidine with a substitution at the C-5 position is an example of a modified nucleotide. Modifications can also include 3′ and 5′ modifications, such as capping. Other modifications can include substitution of one or more of the naturally occurring nucleotides with an analog, internucleoside modifications such as, for example, those with uncharged linkages (e.g., methyl phosphonates, phosphotriesters, phosphoamidates, carbamates, etc.) and those with charged linkages (e.g., phosphorothioates, phosphorodithioates, etc.), those with intercalators (e.g., acridine, psoralen, etc.), those containing chelators (e.g., metals, radioactive metals, boron, oxidative metals, etc.), those containing alkylators, and those with modified linkages (e.g., alpha anomeric nucleic acids, etc.). Further, any of the hydroxyl groups ordinarily present on the sugar of a nucleotide may be replaced by a phosphonate group or a phosphate group; protected by standard protecting groups; or activated to prepare additional linkages to additional nucleotides or to a solid support. The 5′ and 3′ terminal OH groups can be phosphorylated or substituted with amines, organic capping group moieties of from about 1 to about 20 carbon atoms, polyethylene glycol (PEG) polymers, in some embodiments, ranging from about 10 to about 80 kDa, PEG polymers, in some embodiments, ranging from about 20 to about 60 kDa, or other hydrophilic or hydrophobic biological or synthetic polymers. In one embodiment, modifications are of the C-5 position of pyrimidines. These modifications can be produced through an amide linkage directly at the C-5 position or by other types of linkages.

Polynucleotides can also contain analogous forms of ribose or deoxyribose sugars that are generally known in the art, including 2′-O-methyl-, 2′-O-allyl, 2′-fluoro- or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs and abasic nucleoside analogs such as methyl riboside. As noted above, one or more phosphodiester linkages may be replaced by alternative linking groups. These alternative linking groups include embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (-O-) linkage, aryl, alkenyl, cycloalkyl, cycloalkenyl or araldyl. Not all linkages in a polynucleotide need be identical. Substitution of analogous forms of sugars, purines, and pyrimidines can be advantageous in designing a final product, as can alternative backbone structures like a polyamide backbone, for example.

Modulate: As used herein, “modulate” means to alter, either by increasing or decreasing, the level, stability, processing, and/or activity of a target.

Nucleic Acid: As used herein, “nucleic acid,” “oligonucleotide,” and “polynucleotide” are used interchangeably to refer to a polymer of nucleotides and include DNA, RNA, DNA/RNA hybrids and modified versions of such entities. The terms “polynucleotide,” “oligonucleotide,” and “nucleic acid” include double- or single-stranded molecules as well as triple-helical molecules. The term nucleic acid includes aptamers, but is not limited thereto (i.e., the term includes other polymers of nucleotides).

Nuclease: As used herein, the term “nuclease” refers to an enzyme capable of cleaving the phosphodiester bond between nucleotide subunits of an oligonucleotide. As used herein, the term “endonuclease” refers to an enzyme that cleaves phosphodiester bond(s) at a site internal to the oligonucleotide. As used herein, the term “exonuclease” refers to an enzyme which cleaves phosphodiester bond(s) linking the end nucleotides of an oligonucleotide. Biological fluids typically contain a mixture of both endonucleases and exonucleases. Nuclease Resistant: As used herein, the terms “nuclease resistant” and “nuclease resistance” refer to the reduced ability of an oligonucleotide to serve as a substrate for an endo- or exonuclease, such that, when contacted with such an enzyme, the oligonucleotide is either not degraded or is degraded more slowly or to a lesser extent than a control oligonucleotide of similar length and sequence but lacking one or more modifications of the oligonucleotide whose nuclease resistance is being measured.

Nucleotide: As used herein, the term “nucleotide” refers to a ribonucleotide or a deoxyribonucleotide, or a modified form thereof. Nucleotides include species that include purines (e.g., adenine, hypoxanthine, guanine, and the like) as well as pyrimidines (e.g., cytosine, uracil, thymine, and the like). When a base is indicated as “A”, “C”, “G”, “U”, or “T”, it is intended to encompass both ribonucleotides and deoxyribonucleoties, and modified forms thereof.

Pharmaceutically Acceptable: Pharmaceutically acceptable, as used herein, means approved by a regulatory agency of a federal or a state government or listed in the U.S. Pharmacopoeia or other generally recognized pharmacopoeia for use in animals and, more particularly, in humans.

Pharmaceutically Acceptable Salt: Pharmaceutically acceptable salt of a compound (e.g., aptamer), as used herein, refers to a product that contains the compound and one or more additional pharmaceutically-acceptable atoms or groups bound to the compound through ionic bond(s). In some embodiments, a pharmaceutically acceptable salt is produced by contacting the compound with an acid or a base. A pharmaceutically acceptable salt may include, but is not limited to, acid addition salts including hydrochlorides, hydrobromides, phosphates, sulphates, hydrogen sulphates, alkyl sulphonates, aryl sulphonates, arylalkylsulfonates, acetates, benzoates, citrates, maleates, fumarates, succinates, lactates, and tartrates; alkali metal cations such as Li, Na, K, alkali earth metal salts such as Mg or Ca, or organic amine salts.

Pharmaceutical Composition: Pharmaceutical composition, as used herein, refers to a formulation comprising a compound (such as an aptamer) in a form suitable for administration to an individual. A pharmaceutical composition is typically formulated to be compatible with its intended route of administration. Examples of routes of administration include, but are not limited to, intravitreal, enteral and parenteral, including, e.g., subcutaneous injection or infusion, intravenous injection or infusion, intra-articular injection, intra-artery injection and infusion, intra-aqueous humor injection and implantation, and intra-vitreous injection and implantation.

Protein: As used herein, “protein” is used synonymously with “peptide,” “polypeptide,” or “peptide fragment.” A “purified” polypeptide, protein, peptide, or peptide fragment is substantially free of cellular material or other contaminating proteins from the cell, tissue, or cell-free source from which the purified protein is obtained, or substantially free from chemical precursors or other chemicals when chemically synthesized.

SELEX: The term SELEX, as used herein, refers to generally to the selection for nucleic acids that interact with a target molecule in a desirable manner, for example binding with high affinity to a protein; and the amplification of those selected nucleic acids. SELEX may be used to identify aptamers with high affinity to a specific target molecule. The term SELEX and “SELEX process” may be used interchangeably. In some embodiments, methods of selecting aptamers that bind to RIG-I are provided, comprising: (a) preparing a candidate mixture of nucleic acids, wherein the candidate mixture comprises modified nucleic acids in which at least one pyrimidine in at least one, or in each, nucleic acid of the candidate mixture is chemically modified at the C5-position; (b) contacting the candidate mixture with RIG-I, wherein nucleic acids having an increased affinity to RIG-I relative to other nucleic acids in the candidate mixture bind RIG-I, forming nucleic acid-RIG-I complexes; (c) partitioning the increased affinity nucleic acids from the remainder of the candidate mixture; and (d) amplifying the increased affinity nucleic acids to yield a mixture of nucleic acids enriched in nucleic acid sequences that are capable of binding to RIG-I with increased affinity, whereby an aptamer that binds to RIG-I is identified. In certain embodiments, the method further includes performing a slow off-rate enrichment process.

Sequence Identity: Sequence identity, as used herein, in the context of two or more nucleic acid sequences is a function of the number of identical nucleotide positions shared by the sequences (i.e., % identity=number of identical positions/total number of positions in the shorter of the two sequences being compared×100), taking into account the number of gaps, and the length of each gap that needs to be introduced to optimize alignment of two or more sequences. The comparison of sequences and determination of percent identity between two or more sequences can be accomplished using a mathematical algorithm, such as BLAST and Gapped BLAST programs at their default parameters (e.g., Altschul et al., J. Mol. Biol. 215:403, 1990; see also BLASTN at www.ncbi.nlm.nih.gov/BLAST). For sequence comparisons, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith and Waterman, Adv. Appl. Math., 2:482, 1981, by the homology alignment algorithm of Needleman and Wunsch, J. Mol. Biol., 48:443, 1970, by the search for similarity method of Pearson and Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel, F. M. et al., Current Protocols in Molecular Biology, pub. by Greene Publishing Assoc. and Wiley-Interscience (1987)). As used herein, when describing the percent identity of a nucleic acid, such as an aptamer, the sequence of which is at least, for example, about 95% identical to a reference nucleobase sequence, it is intended that the nucleic acid sequence is identical to the reference sequence except that the nucleic acid sequence may include up to five point mutations per each 100 nucleotides of the reference nucleic acid sequence. In other words, to obtain a desired nucleic acid sequence, the sequence of which is at least about 95% identical to a reference nucleic acid sequence, up to 5% of the nucleotides in the reference sequence may be deleted or substituted with another nucleotide, or some number of nucleotides up to 5% of the total number of nucleotides in the reference sequence may be inserted into the reference sequence (referred to herein as an insertion). These mutations of the reference sequence to generate the desired sequence may occur at the 5′ or 3′ terminal positions of the reference nucleobase sequence or anywhere between those terminal positions, interspersed either individually among nucleotides in the reference sequence or in one or more contiguous groups within the reference sequence.

SOMAmer: As used herein, a “SOMAmer” or Slow Off-Rate Modified Aptamer refers to an aptamer (including an aptamers comprising at least one nucleotide with a hydrophobic modification) with an off-rate (t_{1/2})of ≥30 minutes, ≥60 minutes, ≥90 minutes, ≥120 minutes, ≥150 minutes, ≥180 minutes, ≥210 minutes, or ≥240 minutes. In some embodiments, SOMAmers are generated using the improved SELEX methods described in U.S. Pat. No. 7,947,447, entitled “Method for Generating Aptamers with Improved Off-Rates”.

Target Molecule: Target molecule (or target), as used herein, refers to any compound or molecule having a three dimensional chemical structure other than a polynucleotide upon which an aptamer can act in a desirable manner. Non-limiting examples of a target molecule include a protein, peptide, nucleic acid, carbohydrate, lipid, polysaccharide, glycoprotein, hormone, receptor, antigen, antibody, virus, pathogen, toxic substance, substrate, metabolite, transition state analog, cofactor, inhibitor, drug, dye, nutrient, growth factor, cell, tissue, any portion or fragment of any of the foregoing, etc. Virtually any chemical or biological effector may be a suitable target. Molecules of any size can serve as targets. A target can also be modified in certain ways to enhance the likelihood or strength of an interaction between the target and the nucleic acid. A target may also include any minor variation of a particular compound or molecule, such as, in the case of a protein, for example, minor variations in its amino acid sequence, disulfide bond formation, glycosylation, lipidation, acetylation, phosphorylation, or any other manipulation or modification, including conjugation with a labeling component, which does not substantially alter the identity of the molecule. A “target molecule” or “target” is a set of copies of one type or species of molecule or multimolecular structure that is capable of binding to an aptamer. “Target molecules” or “targets” refer to more than one such set of molecules. In some embodiments, the target molecule is human RIG-I protein.

Therapeutically Effective Amount: As used herein, the term “therapeutically effective amount” generally means the amount necessary to ameliorate at least one symptom of a disorder or condition to be prevented, reduced, or treated as described herein. The phrase “therapeutically effective amount” as it relates to the aptamers of the present disclosure means the aptamer dosage that provides the specific pharmacological response for which the aptamer is administered in a significant number of individuals in need of such treatment. It is emphasized that a therapeutically effective amount of an aptamer that is administered to a particular individual in a particular instance will not always be effective in treating the conditions/diseases described herein, even though such dosage is deemed to be a therapeutically effective amount by those of skill in the art.

The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description.

Further, ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise). Any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, “about” or “consisting essentially of” mean±20% of the indicated range, value, or structure, unless otherwise indicated. As used herein, the terms “include” and “comprise” are open ended and are used synonymously. It should be understood that the terms “a” and “an” as used herein refer to “one or more” of the enumerated components. The use of the alternative (e.g. , “or”) should be understood to mean either one, both, or any combination thereof of the alternatives

Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Exemplary Embodiments

The present invention provides aptamers that specifically bind RIG-I protein (sometimes referred to as “RIG-I aptamers”). In some embodiments, an aptamer that binds RIG-I protein comprises the sequence 5′- PEPSZV-3′ (SEQ ID NO: 49), wherein each P is independently, and for each occurrence, a C-5 modified pyrimidine; E is a C-5 modified pyrimidine, A, or G; S is a G or C; Z is a C-5 modified pyrimidine or A; and V is a C, A, or G. In some embodiments, an aptamer that binds RIG-I protein comprises the sequence 5′-PEPSFP-3′ (SEQ ID NO: 50), wherein each P is independently, and for each occurrence, a C-5 modified pyrimidine; E is a C-5 modified pyrimidine, A or G; S is a G or C; and F is a C-5 modified pyrimidine, unmodified C, G, or A. In some such embodiments, the aptamer comprises SEQ ID NOs: 49 and 50. In some such embodiments, the aptamer comprises SEQ ID NOs: 49 and 50 and the sequence 5′-AAPGAPGAGG-3′ (SEQ ID NO: 51). In some such embodiments, the aptamer is at least 30, 40, 50, 60, 70, 80, 90, or 100 nucleotides in length.

In some embodiments, an aptamer that binds RIG-I protein comprises the sequence 5′-PGPGPCAₙPGPGPPPZAZQQCₙZMGPPAAPGAPGAGG -3′ (SEQ ID NO: 52), wherein P is independently, and for each occurrence, a C-5 modified pyrimidine; Z is independently, and for each occurrence, a C-5 modified pyrimidine or A; Q is independently, and for each occurrence, a C-5 modified pyrimidine or G; M is a C or A; and n is independently, and for each occurrence, 0 or 1.

In some embodiments, each C-5 modified pyrimidine containing nucleoside is independently selected from 5-(N-benzylcarboxyamide)-2′-deoxyuridine (BndU), 5-(N-benzylcarboxyamide)-2′-O-methyluridine, 5-(N-benzylcarboxyamide)-2′-fluorouridine, 5-(N-phenethylcarboxyamide)-2′-deoxyuridine (PEdU), 5-(N-thiophenylmethylcarboxyamide)-2′-deoxyuridine (ThdU), 5-(N-isobutylcarboxyamide)-2′-deoxyuridine (iBudU), 5-(N-tyrosylcarboxyamide)-2′-deoxyuridine (TyrdU), 5-(N-3,4-methylenedioxybenzylcarboxyamide)-2′-deoxyuridine (MBndU), 5-(N-4-fluorobenzylcarboxyamide)-2′-deoxyuridine (FBndU), 5-(N-3-phenylpropylcarboxyamide)-2′-deoxyuridine (PPdU), 5-(N-imidizolylethylcarboxyamide)-2′-deoxyuridine (ImdU), 5-(N-isobutylcarboxyamide)-2′-O-methyluridine, 5-(N-isobutylcarboxyamide)-2′-fluorouridine, 5-(N-tryptaminocarboxyamide)-2′-deoxyuridine (TrpdU), 5-(N-R-threoninylcarboxyamide)-2′-deoxyuridine (ThrdU), 5-(N-tryptaminocarboxyamide)-2′-O-methyluridine, 5-(N-tryptaminocarboxyamide)-2′-fluorouridine, 5-(N-[1-(3-trimethylamonium) propyl]carboxyamide)-2′-deoxyuridine chloride, 5-(N-naphthylmethylcarboxyamide)-2′-deoxyuridine (NapdU), 5-(N-naphthylmethylcarboxyamide)-2′-O-methyluridine, 5-(N-naphthylmethylcarboxyamide)-2′-fluorouridine, 5-(N-[1-(2,3-dihydroxypropyl)]carboxyamide)-2′-deoxyuridine), 5-(N-2-naphthylmethylcarboxyamide)-2′-deoxyuridine (2NapdU), 5-(N-2-naphthylmethylcarboxyamide)-2′-O-methyluridine, 5-(N-2-naphthylmethylcarboxyamide)-2′-fluorouridine, 5-(N-1-naphthylethylcarboxyamide)-2′-deoxyuridine (NEdU), 5-(N-1-naphthylethylcarboxyamide)-2′-O-methyluridine, 5-(N-1-naphthylethylcarboxyamide)-2′-fluorouridine, 5-(N-2-naphthylethylcarboxyamide)-2′-deoxyuridine (2NEdU), 5-(N-2-naphthylethylcarboxyamide)-2′-O-methyluridine, 5-(N-2-naphthylethylcarboxyamide)-2′-fluorouridine, 5-(N-3-benzofuranylethylcarboxyamide)-2′-deoxyuridine (BFdU), 5-(N-3-benzofuranylethylcarboxyamide)-2′-O-methyluridine, 5-(N-3-benzofuranylethylcarboxyamide)-2′-fluorouridine, 5-(N-3-benzothiophenylethylcarboxyamide)-2′-deoxyuridine (BTdU), 5-(N-3-benzothiophenylethylcarboxyamide)-2′-O-methyluridine, and 5-(N-3-benzothiophenylethylcarboxyamide)-2′-fluorouridine.

In some embodiments, each C-5 modified pyrimidine containing nucleoside is 5-(N-3-phenylpropylcarboxyamide)-2′-deoxyuridine (PPdU).

In some embodiments, the aptamer comprises one or more sequences selected from SEQ ID NOs : 65-67.

In some embodiments, an aptamer that binds RIG-I protein comprises a sequence that is at least 80%, at least 85%, at least 90%, at least 95%, at least 97%, at least 99%, or 100% identical to a sequence selected from SEQ ID NOs: 4-47 and 68-99 .

In some embodiments, the aptamer is from 35 to 60 nucleotides in length, or from 35 to 50 nucleotides in length, or from 40 to 50 nucleotides in length.

In some embodiments, the RIG-I protein that the aptamer binds is a human RIG-I protein.

In any of the embodiments described herein, the aptamer may be from 35 to 60 nucleotides in length, or from 35 to 50 nucleotides in length, or from 40 to 50 nucleotides in length.

In some embodiments, the RIG-I aptamer may include up to 100 nucleotides, up to 95 nucleotides, up to 90 nucleotides, up to 85 nucleotides, up to 80 nucleotides, up to 75 nucleotides, up to 70 nucleotides, up to 65 nucleotides, up to 60 nucleotides, up to 55 nucleotides, up to 50 nucleotides, up to 45 nucleotides, up to 40 nucleotides, or up to 35 nucleotides.

In another aspect this disclosure, the RIG-I aptamer may have a dissociation constant (K_{d}) for RIG-I of about 10 nM or less. In another exemplary embodiment, the RIG-I aptamer has a dissociation constant (K_{d}) for the RIG-I protein of about 15 nM or less. In yet another exemplary embodiment, the RIG-I aptamer has a dissociation constant (K_{d}) for the RIG-I protein of about 20 nM or less. In yet another exemplary embodiment, the RIG-I aptamer has a dissociation constant (K_{d}) for the RIG-I protein of about 25 nM or less. In yet another exemplary embodiment, the RIG-I aptamer has a dissociation constant (K_{d}) for the RIG-I protein of about 30 nM or less. In yet another exemplary embodiment, the RIG-I aptamer has a dissociation constant (K_{d}) for the RIG-I protein of about 35 nM or less. In yet another exemplary embodiment, the RIG-I aptamer has a dissociation constant (K_{d}) for the RIG-I protein of about 40 nM or less. In yet another exemplary embodiment, the RIG-I aptamer has a dissociation constant (K_{d}) for the RIG-I protein of about 45 nM or less. In yet another exemplary embodiment, the RIG-I aptamer has a dissociation constant (K_{d}) for the RIG-I protein of about 50 nM or less. In yet another exemplary embodiment, the RIG-I aptamer has a dissociation constant (K_{d}) for the RIG-I protein in a range of about 2 pM to about 10 nM (or 2 pM, 3 pM, 4 pM, 5 pM, 6 pM, 7 pM, 8 pM, 9 pM, 10 pM, 15 pM, 20 pM, 25 pM, 30 pM, 35 pM, 40 pM, 45 pM, 50 pM, 60 pM, 70 pM, 80 pM, 90 pM, 100 pM, 150 pM, 200 pM, 250 pM, 300 pM, 350 pM, 400 pM, 450 pM, 500 pM, 550 pM, 600 pM, 650 pM, 700 pM, 750 pM, 800 pM, 850 pM, 900 pM, 950 pM, 1000 pM, 2 nM, 3 nM, 4 nM, 5 nM, 6 nM, 7 nM, 8 nM, 9 nM or 10 nM). In yet another exemplary embodiment, the RIG-I aptamer has a dissociation constant (K_{d}) for the RIG-I protein in a range of at least 2pM (or at least 2pM, 3 pM, 4 pM, 5 pM, 6 pM, 7 pM, 8 pM, 9 pM, 10 pM, 15 pM, 20 pM, 25 pM, 30 pM, 35 pM, 40 pM, 45 pM, 50 pM, 60 pM, 70 pM, 80 pM, 90 pM, 100 pM, 150 pM, 200 pM, 250 pM, 300 pM, 350 pM, 400 pM, 450 pM, 500 pM, 550 pM, 600 pM, 650 pM, 700 pM, 750 pM, 800 pM, 850 pM, 900 pM, 950 pM, 1000 pM, 2 nM, 3 nM, 4 nM, 5 nM, 6 nM, 7 nM, 8 nM, 9 nM or 10 nM). A suitable dissociation constant can be determined with a binding assay using a multi-point titration and fitting the equation y=(max−min)(Protein)/(K_{d}+Protein)+min as described in Example 2. It is to be understood that the determination of dissociation constants is highly dependent upon the conditions under which they are measured and thus these numbers may vary significantly with respect to factors such as equilibration time, etc.

In any of the embodiments described herein, the aptamer, nucleic acid molecule comprises nucleotides of DNA, RNA or a combination thereof.

### Selex

SELEX generally includes preparing a candidate mixture of nucleic acids, binding of the candidate mixture to the desired target molecule to form an affinity complex, separating the affinity complexes from the unbound candidate nucleic acids, separating and isolating the nucleic acid from the affinity complex, purifying the nucleic acid, and identifying a specific aptamer sequence. The process may include multiple rounds to further refine the affinity of the selected aptamer. The process can include amplification steps at one or more points in the process. See, e.g., U.S. Pat. No. 5,475,096, entitled “Nucleic Acid Ligands”. The SELEX process can be used to generate an aptamer that covalently binds its target as well as an aptamer that non-covalently binds its target. See, e.g., U.S. Pat. No. 5,705,337 entitled “Systematic Evolution of Nucleic Acid Ligands by Exponential Enrichment: Chemi-SELEX.”

The SELEX process can be used to identify high-affinity aptamers containing modified nucleotides that confer improved characteristics on the aptamer, such as, for example, improved in vivo stability or improved delivery characteristics. Examples of such modifications include chemical substitutions at the ribose and/or phosphate and/or base positions. SELEX process-identified aptamers containing modified nucleotides are described in U.S. Pat. No. 5,660,985, entitled “High Affinity Nucleic Acid Ligands Containing Modified Nucleotides”, which describes oligonucleotides containing nucleotide derivatives chemically modified at the 5′- and 2′-positions of pyrimidines. U.S. Pat. No. 5,580,737, see supra, describes highly specific aptamers containing one or more nucleotides modified with 2′-amino (2′-NH2), 2′-fluoro (2′-F), and/or 2′-O-methyl (2′-OMe). See also, U.S. Patent Application Publication 20090098549, entitled “SELEX and PHOTOSELEX”, which describes nucleic acid libraries having expanded physical and chemical properties and their use in SELEX and photoSELEX.

SELEX can also be used to identify aptamers that have desirable off-rate characteristics. See U.S. Patent Application Publication 20090004667, entitled “Method for Generating Aptamers with Improved Off-Rates”, which describes improved SELEX methods for generating aptamers that can bind to target molecules. As mentioned above, these slow off-rate aptamers are known as “SOMAmers.” Methods for producing aptamers or SOMAmers and photoaptamers or SOMAmers having slower rates of dissociation from their respective target molecules are described. The methods involve contacting the candidate mixture with the target molecule, allowing the formation of nucleic acid-target complexes to occur, and performing a slow off-rate enrichment process wherein nucleic acid-target complexes with fast dissociation rates will dissociate and not reform, while complexes with slow dissociation rates will remain intact. Additionally, the methods include the use of modified nucleotides in the production of candidate nucleic acid mixtures to generate aptamers or SOMAmers with improved off-rate performance.

A variation of this assay employs aptamers that include photoreactive functional groups that enable the aptamers to covalently bind or “photocrosslink” their target molecules. See, e.g., U.S. Pat. No. 6,544,776 entitled “Nucleic Acid Ligand Diagnostic Biochip”. These photoreactive aptamers are also referred to as photoaptamers. See, e.g., U.S. Pat. No. 5,763,177, U.S. Pat. No. 6,001,577, and U.S. Pat. No. 6,291,184, each of which is entitled “Systematic Evolution of Nucleic Acid Ligands by Exponential Enrichment: Photoselection of Nucleic Acid Ligands and Solution SELEX”; see also, e.g., U.S. Pat. No. 6,458,539, entitled “Photoselection of Nucleic Acid Ligands”. After the microarray is contacted with the sample and the photoaptamers have had an opportunity to bind to their target molecules, the photoaptamers are photoactivated, and the solid support is washed to remove any non-specifically bound molecules. Harsh wash conditions may be used, since target molecules that are bound to the photoaptamers are generally not removed, due to the covalent bonds created by the photoactivated functional group(s) on the photoaptamers.

In both of these assay formats, the aptamers or SOMAmers are immobilized on the solid support prior to being contacted with the sample. Under certain circumstances, however, immobilization of the aptamers or SOMAmers prior to contact with the sample may not provide an optimal assay. For example, pre-immobilization of the aptamers or SOMAmers may result in inefficient mixing of the aptamers or SOMAmers with the target molecules on the surface of the solid support, perhaps leading to lengthy reaction times and, therefore, extended incubation periods to permit efficient binding of the aptamers or SOMAmers to their target molecules. Further, when photoaptamers or photoSOMAmers are employed in the assay and depending upon the material utilized as a solid support, the solid support may tend to scatter or absorb the light used to affect the formation of covalent bonds between the photoaptamers or photoSOMAmers and their target molecules. Moreover, depending upon the method employed, detection of target molecules bound to their aptamers or photoSOMAmers can be subject to imprecision, since the surface of the solid support may also be exposed to and affected by any labeling agents that are used. Finally, immobilization of the aptamers or SOMAmers on the solid support generally involves an aptamer or SOMAmer-preparation step (i.e., the immobilization) prior to exposure of the aptamers or SOMAmers to the sample, and this preparation step may affect the activity or functionality of the aptamers or SOMAmers.

SOMAmer assays that permit a SOMAmer to capture its target in solution and then employ separation steps that are designed to remove specific components of the SOMAmer-target mixture prior to detection have also been described (see U.S. Patent Application Publication 20090042206, entitled “Multiplexed Analyses of Test Samples”). The described SOMAmer assay methods enable the detection and quantification of a non-nucleic acid target (e.g., a protein target) in a test sample by detecting and quantifying a nucleic acid (i.e., a SOMAmer). The described methods create a nucleic acid surrogate (i.e., the SOMAmer) for detecting and quantifying a non-nucleic acid target, thus allowing the wide variety of nucleic acid technologies, including amplification, to be applied to a broader range of desired targets, including protein targets.

Embodiments of the SELEX process in which the target is a peptide are described in U.S. Pat. No. 6,376,190, entitled “Modified SELEX Processes Without Purified Protein.” In the instant case, the target is the RIG-I-Protein.

### Chemical Modifications in Aptamers

Aptamers may contain modified nucleotides that improve its properties and characteristics. Non-limiting examples of such improvements include, in vivo stability, stability against degradation, binding affinity for its target, and/or improved delivery characteristics.

Examples of such modifications include chemical substitutions at the ribose and/or phosphate and/or base positions of a nucleotide. SELEX process-identified aptamers containing modified nucleotides are described in U.S. Pat. No. 5,660,985, entitled “High Affinity Nucleic Acid Ligands Containing Modified Nucleotides,” which describes oligonucleotides containing nucleotide derivatives chemically modified at the 5′- and 2′-positions of pyrimidines. U.S. Pat. No. 5,580,737, see supra, describes highly specific aptamers containing one or more nucleotides modified with 2′-amino (2′—NH₂), 2′-fluoro (2′-F), and/or 2′-O-methyl (2′-OMe). See also, U.S. Patent Application Publication No. 20090098549, entitled “SELEX and PHOTOSELEX,” which describes nucleic acid libraries having expanded physical and chemical properties and their use in SELEX and photoSELEX.

Specific examples of nucleosides comprising a C-5 modification include substitution of deoxyuridine at the C-5 position with a substituent independently selected from: benzylcarboxyamide (alternatively benzylaminocarbonyl) (Bn), naphthylmethylcarboxyamide (alternatively naphthylmethylaminocarbonyl) (Nap), tryptaminocarboxyamide (alternatively tryptaminocarbonyl) (Trp), and isobutylcarboxyamide (alternatively isobutylaminocarbonyl) (iBu) as illustrated immediately below.

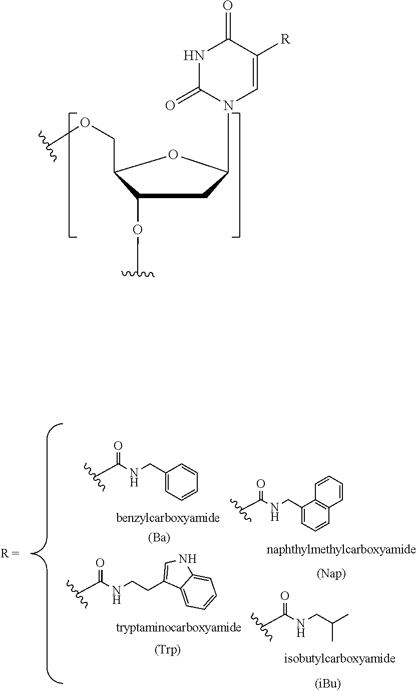

Chemical modifications of a C-5 modified pyrimidine can also be combined with, singly or in any combination, 2′-position sugar modifications, modifications at exocyclic amines, and substitution of 4-thiouridine and the like.

Representative C-5 modified pyrimidine containing nucleosides include: 5-(N-benzylcarboxyamide)-2′-deoxyuridine (BndU), 5-(N-benzylcarboxyamide)-2′-O-methyluridine, 5-(N-benzylcarboxyamide)-2′-fluorouridine, 5-(N-isobutylcarboxyamide)-2′-deoxyuridine (iBudU), 5-(N-isobutylcarboxyamide)-2′-O-methyluridine, 5-(N-isobutylcarboxyamide)-2′-fluorouridine, 5-(N-tryptaminocarboxyamide)-2′-deoxyuridine (TrpdU), 5-(N-tryptaminocarboxyamide)-2′-O-methyluridine, 5-(N-tryptaminocarboxyamide)-2′-fluorouridine, 5- (N-[1- (3-trimethylamonium) propyl] carboxyamide)-2′-deoxyuridine chloride, 5-(N-naphthylmethylcarboxyamide)-2′-deoxyuridine (NapdU), 5-(N-naphthylmethylcarboxyamide)-2′-O-methyluridine, 5-(N-naphthylmethylcarboxyamide)-2′-fluorouridine or 5-(N-[1-(2,3-dihydroxypropyl)]carboxyamide)-2′-deoxyuridine).

If present, a modification to the nucleotide structure can be imparted before or after assembly of the polynucleotide. A sequence of nucleotides can be interrupted by non-nucleotide components. A polynucleotide can be further modified after polymerization, such as by conjugation with a labeling component.

Further, C-5 modified pyrimidine containing nucleotides include the following:

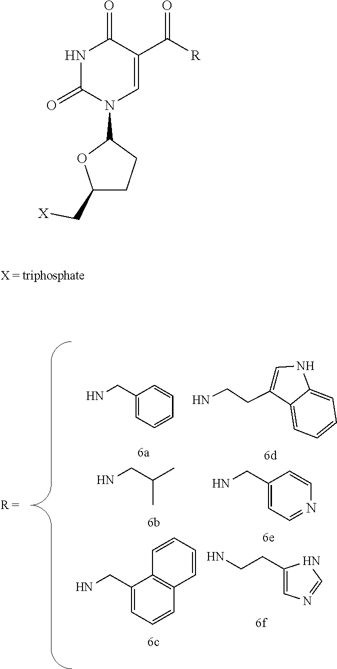

In some embodiments, the modified nucleotide confers nuclease resistance to the oligonucleotide. A pyrimidine with a substitution at the C-5 position is an example of a modified nucleotide. Modifications can include backbone modifications, methylations, unusual base-pairing combinations such as the isobases isocytidine and isoguanidine, and the like. Modifications can also include 3′ and 5′ modifications, such as capping. Other modifications can include substitution of one or more of the naturally occurring nucleotides with an analog, internucleoside modifications such as, for example, those with uncharged linkages (e.g., methyl phosphonates, phosphotriesters, phosphoamidates, carbamates, etc.) and those with charged linkages (e.g. , phosphorothioates, phosphorodithioates, etc.), those with intercalators (e.g., acridine, psoralen, etc.), those containing chelators (e.g., metals, radioactive metals, boron, oxidative metals, etc.), those containing alkylators, and those with modified linkages (e.g., alpha anomeric nucleic acids, etc.). Further, any of the hydroxyl groups ordinarily present on the sugar of a nucleotide may be replaced by a phosphonate group or a phosphate group; protected by standard protecting groups; or activated to prepare additional linkages to additional nucleotides or to a solid support. The 5′ and 3′ terminal OH groups can be phosphorylated or substituted with amines, organic capping group moieties of from about 1 to about 20 carbon atoms, polyethylene glycol (PEG) polymers in one embodiment ranging from about 10 to about 80 kDa, PEG polymers in another embodiment ranging from about 20 to about 60 kDa, or other hydrophilic or hydrophobic biological or synthetic polymers. In one embodiment, modifications are of the C-5 position of pyrimidines. These modifications can be produced through an amide linkage directly at the C-5 position or by other types of linkages.

Polynucleotides can also contain analogous forms of ribose or deoxyribose sugars that are generally known in the art, including 2′-O-methyl-, 2′-O-allyl, 2′-fluoro- or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs and abasic nucleoside analogs such as methyl riboside. As noted above, one or more phosphodiester linkages may be replaced by alternative linking groups. These alternative linking groups include embodiments wherein phosphate is replaced by P(0)S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. Substitution of analogous forms of sugars, purines, and pyrimidines can be advantageous in designing a final product, as can alternative backbone structures like a polyamide backbone, for example.

The present disclosure further provides for a formulation comprising two or more a nucleic acid sequences selected from the group consisting of SEQ ID NOs: 4-47 and 68-99.

In another aspect, each C-5 modified pyrimidine containing nucleoside is independently selected from: 5-(N-benzylcarboxyamide)-2′-deoxyuridine (BndU), 5-(N-benzylcarboxyamide)-2′-O-methyluridine, 5-(N-benzylcarboxyamide)-2′-fluorouridine, 5-(N-phenethylcarboxyamide)-2′-deoxyuridine (PEdU), 5-(N-thiophenylmethylcarboxyamide)-2′-deoxyuridine (ThdU), 5-(N-isobutylcarboxyamide)-2′-deoxyuridine (iBudU), 5-(N-tyrosylcarboxyamide)-2′-deoxyuridine (TyrdU), 5-(N-3,4-methylenedioxybenzylcarboxyamide)-2′-deoxyuridine (MBndU), 5-(N-4-fluorobenzylcarboxyamide)-2′-deoxyuridine (FBndU), 5-(N-3-phenylpropylcarboxyamide)-2′-deoxyuridine (PPdU), 5-(N-imidizolylethylcarboxyamide)-2′-deoxyuridine (ImdU), 5-(N-isobutylcarboxyamide)-2′-O-methyluridine, 5-(N-isobutylcarboxyamide)-2′-fluorouridine, 5-(N-tryptaminocarboxyamide)-2′-deoxyuridine (TrpdU), 5-(N-R-threoninylcarboxyamide)-2′-deoxyuridine (ThrdU), 5-(N-tryptaminocarboxyamide)-2′-O-methyluridine, 5-(N-tryptaminocarboxyamide)-2′-fluorouridine, 5-(N-[1-(3-trimethylamonium) propyl]carboxyamide)-2′-deoxyuridine chloride, 5-(N-naphthylmethylcarboxyamide)-2′-deoxyuridine (NapdU), 5-(N-naphthylmethylcarboxyamide)-2′-O-methyluridine, 5-(N-naphthylmethylcarboxyamide)-2′-fluorouridine, 5-(N-[1-(2,3-dihydroxypropyl)]carboxyamide)-2′-deoxyuridine), 5-(N-2-naphthylmethylcarboxyamide)-2′-deoxyuridine (2NapdU), 5-(N-2-naphthylmethylcarboxyamide)-2′-O-methyluridine, 5-(N-2-naphthylmethylcarboxyamide)-2′-fluorouridine, 5-(N-1-naphthylethylcarboxyamide)-2′-deoxyuridine (NEdU), 5-(N-1-naphthylethylcarboxyamide)-2′-O-methyluridine, 5-(N-1-naphthylethylcarboxyamide)-2′-fluorouridine, 5-(N-2-naphthylethylcarboxyamide)-2′-deoxyuridine (2NEdU), 5-(N-2-naphthylethylcarboxyamide)-2′-O-methyluridine, 5-(N-2-naphthylethylcarboxyamide)-2′-fluorouridine, 5-(N-3-benzofuranylethylcarboxyamide)-2′-deoxyuridine (BFdU), 5-(N-3-benzofuranylethylcarboxyamide)-2′-O-methyluridine, 5-(N-3-benzofuranylethylcarboxyamide)-2′-fluorouridine, 5-(N-3-benzothiophenylethylcarboxyamide)-2′-deoxyuridine (BTdU), 5-(N-3-benzothiophenylethylcarboxyamide)-2′-O-methyluridine, and 5-(N-3-benzothiophenylethylcarboxyamide)-2′-fluorouridine.

In another aspect, the C-5 modified pyrimidine containing nucleoside is independently selected from: 5-(N-1-naphthylmethylcarboxyamide)-2′-deoxyuridine (NapdU), 5-(N-1-naphthylmethylcarboxyamide)-2′-O-methyluridine, 5-(N-1-naphthylmethylcarboxyamide)-2′-fluorouridine, 5-(N-2-naphthylmethylcarboxyamide)-2′-deoxyuridine (2NapdU), 5-(N-2-naphthylmethylcarboxyamide)-2′-O-methyluridine, 5-(N-2-naphthylmethylcarboxyamide)-2′-fluorouridine, 5-(N-1-naphthylethylcarboxyamide)-2′-deoxyuridine (NEdU), 5-(N-1-naphthylethylcarboxyamide)-2′-O-methyluridine, 5-(N-1-naphthylethylcarboxyamide)-2′-fluorouridine, 5-(N-2-naphthylethylcarboxyamide)-2′-deoxyuridine (2NEdU), 5-(N-2-naphthylethylcarboxyamide)-2′-O-methyluridine, 5-(N-2-naphthylethylcarboxyamide)-2′-fluorouridine, 5-(N-3-benzofuranylethylcarboxyamide)-2′-deoxyuridine (BFdU), 5-(N-3-benzofuranylethylcarboxyamide)-2′-O-methyluridine, 5-(N-3-benzofuranylethylcarboxyamide)-2′-fluorouridine, 5-(N-3-benzothiophenylethylcarboxyamide)-2′-deoxyuridine (BTdU), 5-(N-3-benzothiophenylethylcarboxyamide)-2′-O-methyluridine, and 5-(N-3-benzothiophenylethylcarboxyamide)-2′-fluorouridine.

In another aspect, the C-5 modified pyrimidine containing nucleoside is 5-(N-3-phenylpropylcarboxyamide)-2′-deoxyuridine (PPdU).

In another aspect, the two or more nucleic acid molecules of the formulation are each, independently, from 35 to 60 nucleotides in length, or from 35 to 50 nucleotides in length, or from 40 to 50 nucleotides in length; or further comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 additional nucleotides.

In another aspect, the complement component 3 (RIG-I) protein is a human complement component 3 (RIG-I) protein.

### Pharmaceutical Compositions Comprising Aptamers

In some embodiments, pharmaceutical compositions comprising at least one aptamer described herein and at least one pharmaceutically acceptable carrier are provided. Suitable carriers are described in “Remington: The Science and Practice of Pharmacy, Twenty-first Edition,” published by Lippincott Williams & Wilkins, which is incorporated herein by reference.

The aptamers described herein can be utilized in any pharmaceutically acceptable dosage form, including, but not limited to, injectable dosage forms, liquid dispersions, gels, aerosols, ointments, creams, lyophilized formulations, dry powders, tablets, capsules, controlled release formulations, fast melt formulations, delayed release formulations, extended release formulations, pulsatile release formulations, mixed immediate release and controlled release formulations, etc. Specifically, the aptamers described herein can be formulated: (a) for administration selected from any of intravitreal, oral, pulmonary, intravenous, intraarterial, intrathecal, intra- articular, rectal, ophthalmic, colonic, parenteral, intracisternal, intravaginal, intraperitoneal, local, buccal, nasal, and topical administration; (b) into a dosage form selected from any of liquid dispersions, gels, aerosols, ointments, creams, tablets, sachets and capsules; (c) into a dosage form selected from any of lyophilized formulations, dry powders, fast melt formulations, controlled release formulations, delayed release formulations, extended release formulations, pulsatile release formulations, and mixed immediate release and controlled release formulations; or (d) any combination thereof.

Solutions or suspensions used for parenteral, intradermal, or subcutaneous application can comprise one or more of the following components: (1) a sterile diluent such as water for injection, saline solution, fixed oils, polyethylene glycols, glycerine, propylene glycol or other synthetic solvents; (2) antibacterial agents such as benzyl alcohol or methyl parabens; (3) antioxidants such as ascorbic acid or sodium bisulfite; (4) chelating agents such as ethylenediaminetetraacetic acid; (5) buffers such as acetates, citrates or phosphates; and (5) agents for the adjustment of tonicity such as sodium chloride or dextrose. The pH can be adjusted with acids or bases, such as hydrochloric acid or sodium hydroxide. A parenteral preparation can be enclosed in ampoules, disposable syringes or multiple dose vials made of glass or plastic.

Pharmaceutical compositions suitable for injectable use may include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition should be sterile and should be fluid to the extent that easy syringability exists. The pharmaceutical composition should be stable under the conditions of manufacture and storage and should be preserved against the contaminating action of microorganisms such as bacteria and fungi.

The term “stable”, as used herein, means remaining in a state or condition that is suitable for administration to a subject.

The carrier can be a solvent or dispersion medium, including, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol or sorbitol, and inorganic salts such as sodium chloride, in the composition. Prolonged absorption of the injectable compositions can be brought about by including in the composition an agent which delays absorption, for example, aluminum monostearate and gelatin.

Sterile injectable solutions can be prepared by incorporating the active reagent (e.g., an aptamer) in an appropriate amount in an appropriate solvent with one or a combination of ingredients enumerated above, as desired, followed by filtered sterilization. Generally, dispersions are prepared by incorporating at least one aptamer into a sterile vehicle that contains a basic dispersion medium and any other desired ingredient. In the case of sterile powders for the preparation of sterile injectable solutions, exemplary methods of preparation include vacuum drying and freeze-drying, both of which will yield a powder of an aptamer plus any additional desired ingredient from a previously sterile-filtered solution thereof.

In some embodiments, an aptamer is formulated for intravitreal injection. Suitable formulations for intravitreal administration are described, e.g., in “Remington: The Science and Practice of Pharmacy, Twenty-first Edition,” published by Lippincott Williams & Wilkins. Ocular drug delivery is discussed, e.g., in Rawas-Qalaji et al. (2012) Curr. Eye Res. 37: 345; Bochot et al. (2012) J. Control Release 161:628; Yasukawa et al. (2011) Recent Pat. Drug Deliv. Formul. 5:1; and Doshi et al. (2011) Semin. Ophthalmol. 26:104. In some embodiments, a pharmaceutical composition comprising an aptamer is administered by intravitreal injection once per week, once per two weeks, once per three weeks, once per four weeks, once per five weeks, once per six weeks, once per seven weeks, once per eight weeks, once per nine weeks, once per 10 weeks, once per 11 weeks, once per 12 weeks, or less often than once per 12 weeks.

Oral compositions generally include an inert diluent or an edible carrier. They can be enclosed, for example, in gelatin capsules or compressed into tablets. For the purpose of oral therapeutic administration, the aptamer can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition.

For administration by inhalation, the compounds are delivered in the form of an aerosol spray from a pressured container or dispenser that contains a suitable propellant, e.g., a gas such as carbon dioxide, a nebulized liquid, or a dry powder from a suitable device. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active reagents are formulated into ointments, salves, gels, or creams, as generally known in the art. The reagents can also be prepared in the form of suppositories (e.g., with conventional suppository bases such as cocoa butter and other glycerides) or retention enemas for rectal delivery.

In some embodiments, an aptamer is prepared with a carrier that will protect against rapid elimination from the body. For example, a controlled release formulation can be used, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc.

Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Additionally, suspensions of an aptamer may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also include suitable stabilizers or agents to increase the solubility of the compounds and allow for the preparation of highly concentrated solutions.

In some cases, it may be especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of an aptamer calculated to produce the desired therapeutic effect in association with the required pharmaceutical carrier. The specification for the dosage unit forms of aptamers described herein are dictated by and directly dependent on the characteristics of the particular aptamer and the particular therapeutic effect to be achieved, and the limitations inherent in the art of compounding such an active agent for the treatment of individuals.

Pharmaceutical compositions comprising at least one aptamer can include one or more pharmaceutical excipients. Examples of such excipients include, but are not limited to, binding agents, filling agents, lubricating agents, suspending agents, sweeteners, flavoring agents, preservatives, buffers, wetting agents, disintegrants, effervescent agents, and other excipients. Such excipients are known in the art. Exemplary excipients include: (1) binding agents which include various celluloses and cross-linked polyvinylpyrrolidone, microcrystalline cellulose, such as Avicel PH101 and Avicel PHI 02, silicified microcrystalline cellulose (ProSolv SMCCTM), gum tragacanth and gelatin; (2) filling agents such as various starches, lactose, lactose monohydrate, and lactose anhydrous; (3) disintegrating agents such as alginic acid, Primogel, corn starch, lightly crosslinked polyvinyl pyrrolidone, potato starch, maize starch, and modified starches, croscarmellose sodium, cross-povidone, sodium starch glycolate, and mixtures thereof; (4) lubricants, including agents that act on the flowability of a powder to be compressed, and including magnesium stearate, colloidal silicon dioxide, such as Aerosil 200, talc, stearic acid, calcium stearate, and silica gel; (5) glidants such as colloidal silicon dioxide; (6) preservatives, such as potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride; (7) diluents such as pharmaceutically acceptable inert fillers, such as microcrystalline cellulose, lactose, dibasic calcium phosphate, saccharides, and/or mixtures of any of the foregoing; examples of diluents include microcrystalline cellulose, such as Avicel PH101 and Avicel PHI 02; lactose such as lactose monohydrate, lactose anhydrous, and Pharmatose DCL21 ; dibasic calcium phosphate such as Emcompress ; mannitol; starch; sorbitol; sucrose; and glucose; (8) sweetening agents, including any natural or artificial sweetener, such as sucrose, saccharin sucrose, xylitol, sodium saccharin, cyclamate, aspartame, and acesulfame; (9) flavoring agents, such as peppermint, methyl salicylate, orange flavoring, Magnasweet (trademark of MAFCO), bubble gum flavor, fruit flavors, and the like; and (10) effervescent agents, including effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

In various embodiments, the formulations described herein are substantially pure. As used herein, “substantially pure” means the active ingredient (e.g., an aptamer) is the predominant species present (i.e., on a molar basis it is more abundant than any other individual species in the composition). In some embodiments, a substantially purified fraction is a composition wherein the active ingredient comprises at least about 50 percent (on a molar basis) of all macromolecular species present. Generally, a substantially pure composition will include more than about 80% of all macromolecular species present in the composition. In various embodiments, a substantially pure composition will include at least about 85%, at least about 90%, at least about 95%, or at least about 99% of all macromolecular species present in the composition. In various embodiments, the active ingredient is purified to homogeneity (contaminant species cannot be detected in the composition by conventional detection methods) wherein the composition consists essentially of a single macromolecular species.

### Kits Comprising Aptamers

The present disclosure provides kits comprising any of the aptamers described herein. Such kits can comprise, for example, (1) at least one aptamer; and (2) at least one pharmaceutically acceptable carrier, such as a solvent or solution. Additional kit components can optionally include, for example: (1) any of the pharmaceutically acceptable excipients identified herein, such as stabilizers, buffers, etc., (2) at least one container, vial or similar apparatus for holding and/or mixing the kit components; and (3) delivery apparatus.

The following examples are provided to illustrate certain particular features and/or embodiments. These examples should not be construed to limit the disclosure to the particular features or embodiments described.

### EXAMPLES

### Example 1

### Selection and Identification of Aptamers Having Binding Specificity to RIG-I Protein

This example provides the representative method for the selection and production of DNA aptamers to the human RIG-I protein.

Preparation of Candidate Mixture

A candidate mixture of partially randomized ssDNA oligonucleotides was prepared by polymerase extension of a DNA primer annealed to a biotinylated ssDNA template (shown in Table 1 below). The candidate mixture contained a 40 nucleotide randomized cassette containing dATP, dGTP, dCTP and 5-(N-3-phenylpropylcarboxyamide)-2′-deoxyuridine triphosphate (PPdUTP).

TABLE 1
Sequences of Template and Primers

| Oligonucleotide | | SEQ |
|---|---|---|
| Designation | Sequence (5′ → 3′) | ID NO: |
| Template 1 | AB′AB′TT TTT TTT CCC GTG | 1 |
| | TTG CTG TCT GCC- | |
| | (N)₄₀-GCGGGAGCAGGGTAGAG | |
| Primer 1 | ATATATATCGCCCTCGTCCCATCTC | 2 |
| Primer 2 | AB′AB′ TT TTT TTT CCC GTG | 3 |
| | TTG CTG TCT GCC | |

B′ = biotin

Seven hundred fifty microliters of a 50% slurry of Streptavidin Plus UltraLink Resin (PIERCE) was washed once with 3.5 mL of SB18T0.01 (40 mM HEPES (4-(2-Hydroxyethyl)piperazine-1-ethanesulfonic acid) buffer adjusted to pH 7.5 with NaOH, 102 mM NaCl, 5 mM KCl, 5 mM MgCl2 and 0.01% TWEEN 20) and thrice with 3.5 mL of 16 mM NaCl. Thirty nanomoles of template 1 (SEQ ID NO: 1) possessing two biotin residues (designated as B′ in the sequence) and 40 randomized positions (designated as N40 in the sequence) were added to the washed UltraLink SA beads and rotated at 37° C. for 30 minutes. The beads were then washed three times with 16 mM NaCl. Between each wash, the beads were recovered by centrifugation. The beads, now containing the captured template, were suspended in a 0.75 mL of extension reaction buffer [containing 20 nmol of primer 1 (SEQ ID NO: 2), 1X SQ20 buffer (120 mM Tris-HCl, pH7.8, 10 mM KCl, 7 mM MgSO₄, 6 mM (NH₄)₂SO₄, 0.001% BSA and 0.1% Triton X-100), 75 units of KOD XL DNA Polymerase (EMD MILLIPORE), and 0.5 mM each of dATP, dCTP, dGTP and PPdUTP. The beads were allowed to incubate at 68° C. for 2 hours. The beads were then washed three times with 16 mM NaCl. The aptamer library was eluted from the beads with 0.5 mL of 20 mM NaOH. The eluted library and immediately neutralized with 15 μL of 1N HCl and 10 μL HEPES pH 7.5 and 1 μL 10% TWEEN-20. The library was concentrated with an AMICON Ultracel YM-10 filter to approximately 0.09 mL and the concentration of library determine by ultraviolet absorbance spectroscopy.

### Biotin Labeling of Human RIG-I Protein

Untagged human RIG-I C-terminal domain protein (amino acids 792-925, SEQ ID NO: 56) purified from E. coli Rosetta II cells was biotinylated by covalent coupling of NHS-PEO4-biotin (PIERCE, EZ-Link NHS-PEG4-Biotin) to residues containing primary amines. Protein (270 pmol in 14 μL) was mixed with a 22-fold molar excess of NHS-PEG4-biotin and the reaction was allowed to incubate at room temperature for 45 minutes. After the reaction was completed, buffer was exchanged and unreacted NHS-PEG4-biotin removed by ultrafiltration using YM3 filters (MILLIPORE). The exchange buffer was SB18T0.01.

Immobilization of Target Protein

Biotin labeled target protein was immobilized on MyOne-SA paramagnetic beads (MyOne SA, Invitrogen, or hereinafter referred to as SA beads) for Round 1 through Round 3 and Round 7 through 9 of SELEX. Beads (250 mgs) were prepared by washing three times with 25 mL of SB18T0.01. Finally, the beads were suspended at 10 mgs/mL in SB18T0.01 and stored at 4° C. until use.

His-tagged generated target protein was immobilized on His-tag Dynabeads (Thermo Fisher) paramagnetic beads (MyOne SA, Invitrogen, or hereinafter referred to as His beads) for Round 4 through Round 6 of SELEX. Beads (40 mgs) were prepared by washing three times with 20 mL of SB18T0.01. Finally, the beads were suspended at 2.5 mgs/mL in SB18T0.01 and stored at 4° C. until use.

Aptamer Selection with Slow Off-Rate Enrichment Process

A total of nine rounds of the SELEX process were completed with selection for affinity and slow off-rate. Prior to each round a counter selection was performed to reduce background and to reduce the likelihood of obtaining aptamers with nonspecific binding to protein. Counter selections were performed as follows.

For round 1, 100 μL of the DNA candidate mixture containing approximately 1 nmole of DNA in SB18T0.01 was heated at 95° C. for 5 minutes and then cooled to 70° C. for 5 minutes, then to 48° C. for 5 minutes and then transferred to a 37° C. block for 5 minutes. The sample was then combined with 10 μL of protein competitor mixture (0.1% HSA, 10 μM casein, and 10 μM prothrombin in SB18T0.01), and 0.1 mg (10 μL) of SA beads and incubated at 37° C. for 10 minutes with mixing. Beads were removed by magnetic separation.

For Rounds 2-9, a 65 μL aliquot of the DNA candidate mixture obtained from the previous round (65% of eDNA obtained from previous round) was mixed with 16 μL of 5× SB18T0.01. The sample was heated to 95° C. for 3 minutes and cooled to 37° C. at a rate of 0.1° C./second. The sample was then combined with 9 μL of protein competitor mixture (0.1% HSA, 10 μM casein, and 10 μM prothrombin in SB18T0.01), and 0.1 mg (10 μL) of SA beads (rounds 2-3, rounds 7-9) or 0.025 mg (10 uL) His beads (rounds 4-6) and incubated at 37° C. for 10 minutes with mixing. Beads were removed by magnetic separation.

Following the first counter selection the target protein was pre-immobilized on SA beads for the Round 1 selection process. To accomplish this, 0.5 mg of protein SA beads were mixed with 50 pmoles of target protein premixed with 100 pmoles of a small hairpin RNA (herein after referred to as HP10) and incubated for 30 minutes at 37° C. Unbound target was removed by washing the beads with SB18T0.01. The counter-selected-DNA candidate mixture (100 μL) was added to the beads and incubated at 37° C. for 60 minutes with mixing. No slow off-rate enrichment process was employed in the first round and beads were simply washed 2 times with 100 μL biotin wash (25 μM biotin in SB18T0.01) and 3 times with 100 μL SB18T0.01. Following the washes, the bound aptamer was eluted from the beads by adding 85 μL of 2 mM NaOH, and incubating at 37° C. for 5 minutes with mixing. The aptamer—containing-eluate (80 μL) was transferred to a new tube after magnetic separation of the beads and the solution neutralized by addition of 20 μL of neutralization buffer (500 mM Tris-HCl pH 7.5, 8 mM HCl).

For Rounds 2-9, selections were performed with the DNA candidate mixture and target protein as described below while, in parallel, an identical selection was performed with the DNA candidate mixture, but without the target protein. Comparison of the Ct values obtained from PCR for the sample with target protein (signal S) and sample without target protein (background B) were used as a guide to reduce the target concentration in the next round. If the delta Ct value was greater than 4, but less than 8, the target protein was reduced three fold in the next round. If the delta Ct value was greater than 8, the target was reduced 10-fold in the next round.

For Round 2, labeled target protein (5 pmoles in 10 μL) was mixed with 25 pmoles HP10 and 40 μL of counter selected DNA candidate mixture and incubated at 37° C. for 15 minutes. A slow off-rate enrichment process was begun by adding 50 μL of 10 mM dextran sulfate followed by the immediate addition of 0.1 mg of SA beads. This was allowed to incubate for 15 minutes at 37° C. with mixing. Beads were then washed 2 times with 100 biotin wash μL and 3 times with 100 μL of SB18T0.01. The aptamer strand was eluted from the beads by adding 100 μL of sodium perchlorate, and incubating at 37° C. for 10 minutes with mixing. Beads were removed by magnetic separation and 100 μL of aptamer eluate was transferred to a new tube.

Round 3 and rounds 7 through 9 were performed as described for Round 2 except the amount of target protein was 1.6 pmoles for round 7, 0.5 pmoles for round 8 and 0.16 pmoles for round 9. The dextran sulfate was added 10 minutes (round 3), 45 minutes (round 7), 120 minutes (rounds 8 and 9) prior to the addition of SA beads.

Rounds 4 through 6 were performed using His-tagged target protein. For Round 4, target (1.6 pmoles in 10 μL) was mixed 25 pmoles HP10 and 40 μL of counter-selected-DNA candidate mixture and incubated at 37° C. for 15 minutes with mixing. A slow off-rate enrichment process was then begun by adding 50 μL of 10 mM dextran sulfate and the mixture allowed to incubate for an additional 20 minutes with mixing. His beads (0.025 mg) were added in order to capture the target protein-aptamer complexes (15 minutes incubation at 37° C. with mixing). Beads were then washed 5 times with 100 μL of SB18T0.01. Bound aptamers were eluted from the beads by adding 100 μL of sodium perchlorate and incubating at 37° C. for 10 minutes with mixing. Beads were removed by magnetic separation and 100 μL of aptamer eluate was transferred to a new tube.

Rounds 5 and 6 were performed as Round 4 except that a 30 minute dextran challenge was utilized.

For rounds 2 through 9, following the perchlorate elution, 100 μL of the aptamer eluate was captured with 0.0625 mg (25 μL) of SA beads pre-bound with primer 2 (SEQ ID NO: 3), herein after referred to as Primer Beads, and incubated at 50° C. for 10 minutes with shaking followed by a 25° C. incubation for 10 minutes with shaking. The beads were washed 2 times with 100 μL SB18T0.01 and 1 time with 16 mM NaCl. Bound aptamer was eluted with 80 μL water and incubated at 75° C. for 2 minutes. Beads were removed by magnetic separation and 80 μL of aptamer eluate was transferred to a new tube. Primer beads were prepared by resuspending 20 mg SA beads (2 mL of 10 mg/mL SA beads washed once with 2 mL 20 mM NaOH, twice with 2 mL SB18T0.01) in 0.75 mL 1 M NaCl, 0.01% tween-20 and adding 4 nmoles primer 2 (SEQ ID NO: 3). The mixture was incubated at 37° C. for 1 hour. Following incubation, the beads were washed 2 times with 1 mL SB18T0.01 and 2 times with 1 mL 16 mM NaCl. Beads were resuspended to 2.5 mg/ml in 5 M NaCl, 0.01% tween-20.

Aptamer Amplification and Purification

Selected aptamer DNA from each round was amplified and quantified by QPCR. 48 μL DNA was added to 12 μL QPCR Mix (10× KOD DNA Polymerase Buffer; Novagen #71157, diluted to 5×, 25 mM MgCl₂, 5 μM forward PCR primer (Primer 1, SEQ ID NO: 2), 5 μM biotinylated reverse PCR primer (Primer 2, SEQ ID NO: 3), 5× SYBR Green I, 0.075 U/μL KOD XL DNA Polymerase, and 1 mM each dATP, dCTP, dGTP, and dTTP) and thermal cycled in a Bio-Rad MyIQ QPCR instrument with the following protocol: 1 cycle of 96° C. for 15 seconds, 55° C. for 10 seconds, and 71° C. for 30 minutes; followed by 30 cycles of 96° C. for 15 seconds, 71° C. for 1 minute. Quantification was done with the instrument software and the number of copies of DNA selected, with and without target protein, was compared to determine signal/background ratios.

Following amplification, the PCR product was captured on SA beads via the biotinylated antisense strand. 25 mL SA beads (10 mg/mL) were washed once with 25 mL 20 mM NaOH, twice with 25 mL SB18T0.01, resuspended in 25 mL SB18T0.01, and stored at 4° C. 25 μL SA beads (10 mg/mL in SB18T0.01) were added to 50 μL double-stranded QPCR products and incubated at 25° C. for 5 minutes with mixing. The “sense” strand was eluted from the beads by adding 100 μL 20 mM NaOH, and incubating at 25° C. for 1 minute with mixing. The eluted strand was discarded and the beads were washed 2 times with SB18T0.01 and once with 16 mM NaCl.

Aptamer sense strand containing PPdUTP was prepared by primer extension from the immobilized antisense strand. The beads were suspended in 40 μL primer extension reaction mixture (1× Primer Extension Buffer (120 mM Tris-HClpH 7.8, 10 mM KCl, 7 mM MgSO₄, 6 mM (NH₄)₂SO₄, 0.1% TRITON X-100 and 0.001% bovine serum albumin), 3 μM forward primer (Primer 1, SEQ ID NO: 2), 0.5 mM each dATP, dCTP, dGTP, and PPdUTP, and 0.015 U/μL KOD XL DNA Polymerase) and incubated at 71° C. for 30 minutes with mixing. The beads were washed 2 times with SB18T0.01, 1 time with 16 mM NaCl and the aptamer strand was eluted from the beads by adding 85 μL of 20 mM NaOH, and incubating at 37° C. for 1 minute with mixing. 80 μL aptamer eluate was transferred to a new tube after magnetic separation, neutralized with 20 μL of 80 mM HCl, buffered with 5 μL of 0.1 M HEPES, pH 7.5.

Selection Stringency and Feedback

The relative target protein concentration of the selection step was lowered each round in response to the QPCR signal (Δ Ct) following the rule below:

If Δ Ct<4, [P]₍ⱼ₊₁₎=[P]₍ᵢ₎

If 4≤Δ Ct<8, [P]₍ⱼ₊₁₎=[P]₍ᵢ₎/3.2

If Δ Ct≥8, [P]₍ᵢ₊₎=[P]₍ᵢ₎/10

Where [P]=protein concentration and i=current round number.

After each selection round, the convergence state of the enriched DNA mixture was determined. 10 μL double-stranded QPCR product was diluted to 200 μL with 4 mM MgCl₂ containing 1X SYBR Green I. Samples were analyzed for convergence using a C₀t analysis which measures the hybridization time for complex mixtures of double stranded oligonucleotides. Samples were thermal cycled with the following protocol: 3 cycles of 98° C. for 1 minute, 85° C. for 1 minute; 2 cycles of 98° C. for 1 minute, then 85° C. for 30 minutes. During the 30 minutes at 85° C., fluorescent images were measured at 5-second intervals. The fluorescence intensity was plotted as a function of the logarithm of time, and an increased rate of hybridization with each SELEX round was observed, indicating sequence convergence.

Enriched Pool Sequencing & Aptamer Identification

After nine rounds of SELEX, the converged pools from round 7 and round 9 were sequenced. Sequence preparation was performed as follows. The pool was amplified by PCR using SELEX library-specific primers containing a unique barcode/index sequence (unique sequence identifier for each pool). Individual PCR products were quantified using a Quant-iT™ PicoGreen® dsDNA Reagent (Life Technologies) assay, combined at equimolar concentrations, and concentrated/buffer exchanged using an Amicon Ultra-0.5 Centrifugal Filter Device (Millipore). The mixture was then purified by SDS-polyacrylamide gel electrophoresis (PAGE), and the eluate concentrated using an Amicon Ultra-0.5 Centrifugal Filter Device and visualized by PAGE to confirm the size, purity and yield of the final mix. The sample was submitted to SeqWright Genomic Services (GE Healthcare, Houston, Tex.) for Ion Torrent PGM sequencing. From each sequence pool containing over 40,000 sequences, 384 were randomly selected and analyzed for convergence using custom software that determines sequence counts/copy number and identifies common convergence patterns using a local-alignment algorithm. Sequences with the greatest representation/copy number in the pool and at least one sequence from every convergence pattern were chosen for further characterization.

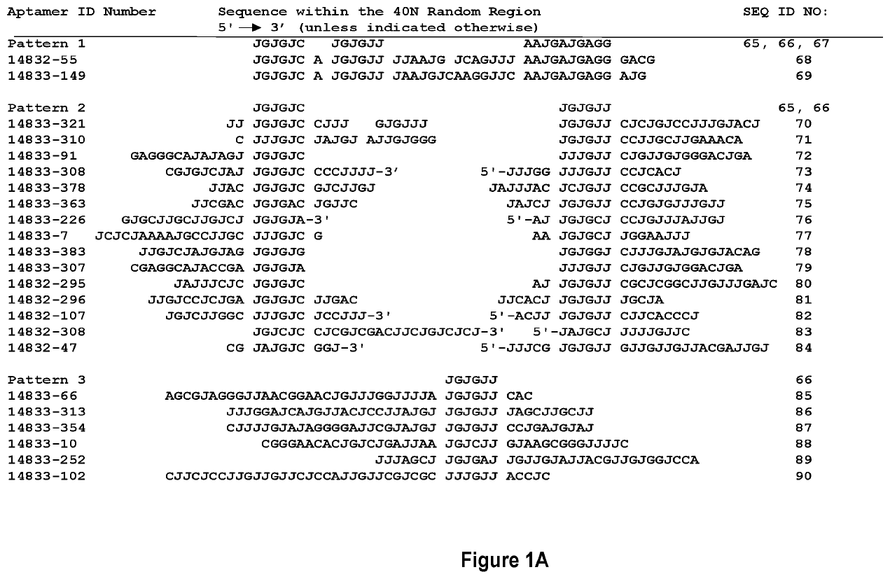

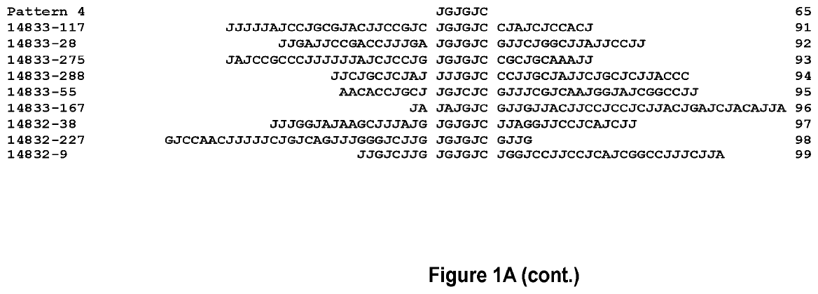

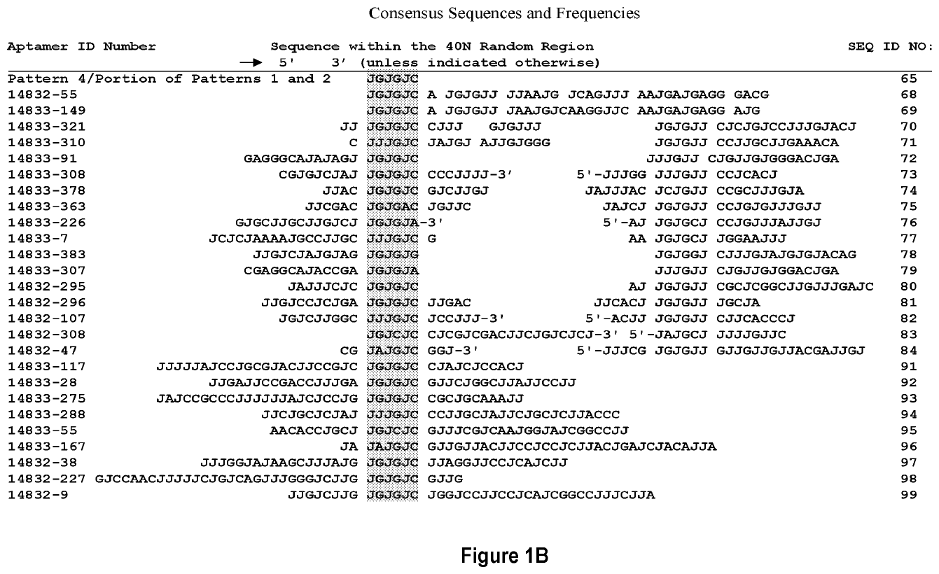

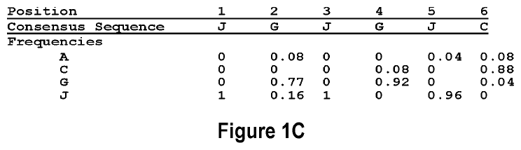

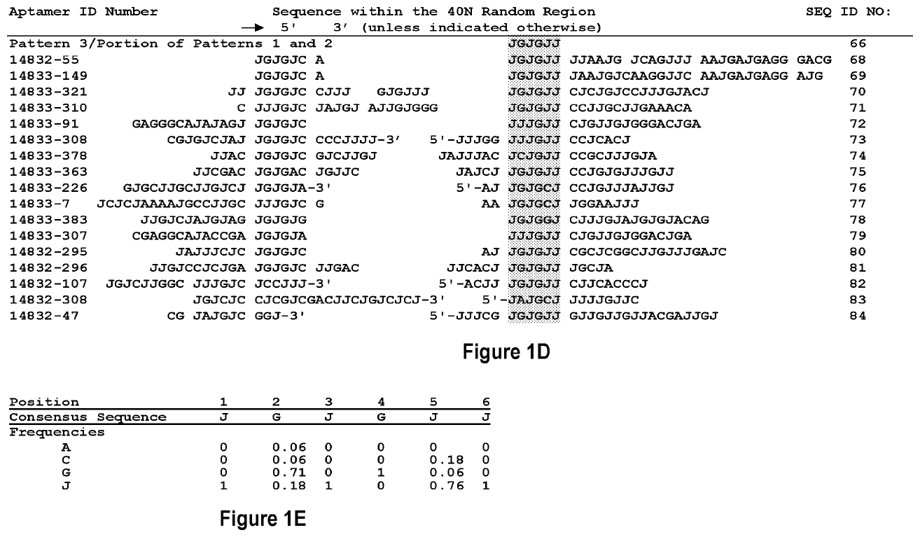

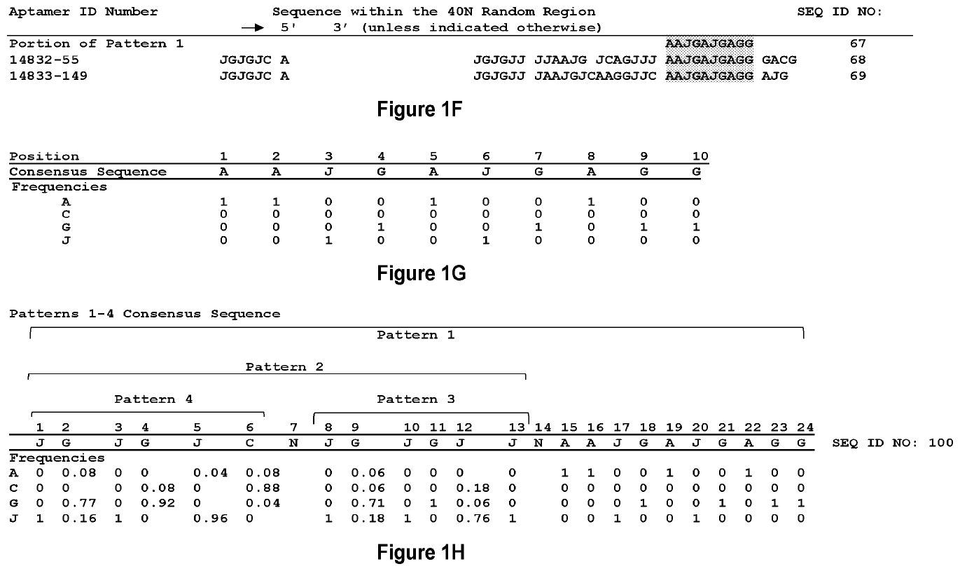

Sequence patterns la and lb were originally identified from the 40 nucleotide randomized regions of sequences 14832-55 (SEQ ID NO: 68), and 14833-149 (SEQ ID NO: 69). Sequences with pattern 1a and their equivalents (differing by less than 5%) contained three conserved regions and represented 4% of the round 7 pool and 13% of the round 9 pool. Additional sequences were identified in the round 7 and round 9 pools that contained two of the three patterns or one of the three patterns found in sequence pattern 1. The sequences of certain aptamers are shown in Table 2 below. Sequence patterns are shown in Table 3 below. Alignments of the sequence patterns with the 40 nucleotide randomized regions of certain aptamers are shown in FIGS. 1A, B, D, and F.

Aptamer Synthesis

For determination of the binding potential, individual aptamers were prepared by solid phase synthesis. The modified deoxyuridine-5-carboxamide amidite reagent used for solid-phase synthesis was prepared by: condensation of 5′-O-(4,4′-dimethoxytrityl)-5-trifluoroethoxycarbonyl-2′-deoxyuridine (Nomura et al. (1997) Nucl. Acids Res. 25:2784) with the appropriate [1-naphthylmethylamine] primary amine (RNH₂, 1.2 eq; Et₃N, 3 eq.; acetonitrile; 60° C.; 4h); 3′-O-phophitidylation with 2-cyanoethyl-N,N-diisopropylchlorophosphoramidite (1.2 eq.; iPr₂EtN, 3 eq.; CH₂Cl₂; −10 to 0° C.; 4 h); and purification by flash chromatography on neutral silica gel (Still, et al. (1978) J. Org. Chem. 43:2923). Aptamers were prepared by solid phase synthesis using the phosphoramidite method (Beaucage and Caruthers (1981) Tetrahedron Lett. 22:1859) with some adjustments to the protocol to account for the unique base modifications described herein. Detritylation was accomplished with 10% dichloroacetic acid in toluene for 45 seconds; coupling was achieved with 0.1 M phosphoramidites in 1:1 acetonitrile:dichloromethane activated by 5-benzylmercaptotetrazole and allowed to react 3 times for 5 minutes; capping and oxidation were performed according to instrument vendor recommendations. Deprotection was affected with gaseous ammonia or methylamine under optimized pressure, time, and temperature in a Parr stainless steel reactor. Products were eluted with dI water into suitable 96-well plates, statistically sampled (√N+1) for LCMS characterization, quantified by UV spectrophotometry, and tested for protein binding affinity in buffered aqueous solution.

TABLE 2

| | Full Sequence (5′ to 3′); | SEQ |
|---|---|---|
| | “J” is 5-(N-3- | ID |
| Aptamer ID | phenylpropylcarboxyamide) uracil | NO. |
| 14832-55_3 | ATCTCJGJGJCAJGJGJJJJAAJGJCAGJJJA | 4 |
| | AJGAJGAGGGACGGGCAG | |
| 14833-10_3 | ATCTCCGGGAACACJGJCJGAJJAAJGJCJJG | 17 |
| | JAAGCGGGJJJJCGGCAG | |
| 14833-149_3 | ATCTCJGJGJCAJGJGJJJAAJGJCAAGGJJC | 18 |
| | AAJGAJGAGGAJGGGCAG | |
| 14833-321_3 | ATCTCJJJGJGJCCJJJGJGJJJJGJGJJCJC | 19 |
| | JGJCCJJJGJACJGGCAG | |
| 14833-310_3 | ATCTCCJJJGJCJAJGJAJJGJGGGJGJGJJC | 20 |
| | CJJGCJJGAAACAGGCAG | |
| 14833-91_3 | ATCTCGAGGGCAJAJAGJJGJGJCJJJGJJCJ | 21 |
| | GJJGJGGGACJGAGGCAG | |
| 14833-308_3 | ATCTCJJJGGJJJGJJCCJCACJCGJGJCJAJ | 22 |
| | JGJGJCCCCJJJJGGCAG | |
| 14833-378_3 | ATCTCJJACJGJGJCGJCJJGJJAJJJACJCJ | 23 |
| | GJJCCGCJJJGJAGGCAG | |
| 14833-363_3 | ATCTCJJCGACJGJGACJGJJCJAJCJJGJGJ | 24 |
| | JCCJGJGJJJGJJGGCAG | |
| 14833-226_3 | ATCTCAJJGJGCJCCJGJJJAJJGJGJGCJJG | 25 |
| | CJJGJCJJGJGJAGGCAG | |
| 14833-7_3 | ATCTCJCJCJAAAAJGCCJJGCJJJGJCGAAJ | 26 |
| | GJGCJJGGAAJJJGGCAG | |
| 14833-383_3 | ATCTCJJGJCJAJGJAGJGJGJGJGJGGJCJJ | 27 |
| | JGJAJGJGJACAGGGCAG | |
| 14833-307_3 | CATCTCCGAGGCAJACCGAJGJGJAJJJGJJC | 28 |
| | JGJJGJGGACJGAGGCAG | |
| 14832-295_3 | ATCTCJAJJJCJCJGJGJCAJJGJGJJCGCJC | 29 |
| | GGCJJGJJJGAJCGGCAG | |
| 14832-296_3 | CATCTCJJGJCCJCJGAJGJGJCJJGACJJCA | 30 |
| | CJJGJGJJJGCJAGGCAG | |
| 14832-107_3 | ATCTCACJJJGJGJJCJJCACCCJJGJCJJGG | 31 |
| | CJJJGJCJCCJJJGGCAG | |
| 14832-308_3 | CATCTCJAJGCJJJJJGJJCJGJCJCCJCGJC | 32 |
| | GACJJCJGJCJCJGGCAG | |
| 14832-47_3 | CATCTCJJJCGJGJGJJGJJGJJGJJACGAJJ | 33 |
| | GJCGJAJGJCGGJGGCAG | |
| 14833-66_3 | ATCTCAGCGJAGGGJJAACGGAACJGJJJGGJ | 34 |
| | JJJAJGJGJJCACGGCAG | |
| 14833-313_3 | ATCTCJJJGGAJCAJGJJACJCCJJAJGJJGJ | 35 |
| | GJJJAGCJJGCJJGGCAG | |
| 14833-354_3 | ATCTCCJJJJGJAJAGGGGAJJCGJAJGJJGJ | 36 |
| | GJJCCJGAJGJAJGGCAG | |
| 14833-252_3 | CCCATCTCJJJAGCJJGJGAJJGJJGJAJJAC | 37 |
| | GJJGJGGJCCAGGCAGAC | |
| 14833-102_3 | TCTCCJJCJCCJJGJJGJJCJCCAJJGJJCGJ | 38 |
| | CGCJJJGJJACCJCGGCA | |
| 14833-117_3 | ATCTCJJJJJAJCCJGCGJACJJCCGJCJGJG | 39 |
| | JCCJAJCJCCACJGGCAG | |
| 14833-28_3 | ATCTCJJGAJJCCGACCJJJGAJGJGJCGJJC | 40 |
| | JGGCJJAJJCCJJGGCAG | |
| 14833-275_3 | ATCTCJAJCCGCCCJJJJJJAJCJCCJGJGJG | 41 |
| | JCCGCJGCAAAJJGGCAG | |
| 14833-288_3 | CATCTCJJCJGCJCJAJJJJGJCCCJJGCJAJ | 42 |
| | JCJGCJCJJACCCGGCAG | |
| 14833-55_3 | CATCTCAACACCJGCJJGJCJCGJJJCGJCAA | 43 |
| | JGGJAJCGGCCJJGGCAG | |
| 14833-167_3 | ATCTCJAJAJGJCGJJGJJACJJCCJCCJCJJ | 44 |
| | ACJGAJCJACAJJAGGCA | |
| 14832-38_3 | ATCTCJJJGGJAJAAGCJJJAJGJGJGJCJJA | 45 |
| | GGJJCCJCAJCJJGGCAG | |
| 14832-227_3 | ATCTCGJCCAACJJJJJCJGJCAGJJJGGGJC | 46 |
| | JJGJGJGJCGJJGGGCAG | |
| 14832-9_3 | ATCTCJJGJCJJGJGJGJCJGGJCCJJCCJCA | 47 |
| | JCGGCCJJJCJJAGGCAG | |

TABLE 3

| Pattern | Sequence(s) (5′ to 3′) | SEQ ID NO. |
|---|---|---|
| 1a | PEPSZV | 49 |
| (3 regions) | PEPSFP | 50 |
| | AAPGAPGAGG | 51 |
| 1b | PGPGPCAₙPGPGPPPZAZQQCₙZ | 52 |
| (1 region) | MGPPAAPGAPGAGG | |
| 2 | PEPSZV | 49 |
| (2 regions) | PEPSFP | 50 |
| 3 | PEPSFP | 50 |
| (1 region) | | |
| 4 | PEPSZV | 49 |
| (1 region) | | |

In Table 3, P is independently, and for each occurrence, a C-5 modified pyrimidine; E is a C-5 modified pyrimidine, A or G; S is a G or C; Z is independently, and for each occurrence a C-5 modified pyrimidine or A; V is a C, A or G; F is a C-5 modified pyrimidine, unmodified C, G or A; Q is independently, and for each occurrence a C-5 modified pyrimidine or G; M is a C or A; and n is independently, and for each occurrence, 0 or 1

### Example 2

### Equilibrium Binding Constant (Kd) for Aptamers to RIG-I Protein

This example provides the method used herein to measure aptamer-RIG-I protein binding affinities and to determine K_{d}. The binding affinities of aptamers 14832-55_3 (SEQ ID NO: 4), 14833-10_3 (SEQ ID NO: 17), and truncations of 14832-55_3, which are shown in Table 4 below, were determined. Briefly, binding constants (K_{d} values) of modified aptamers were determined by electrophoretic mobility shift assay (EMSA) for binding to RIG-I CTD and by filter binding assay for binding to wild type full length RIG-I, RIG-I- mutants, and other RIG-I truncates. RIG-I proteins used for binding assays was purified from E. coli Rosetta II cells. K_{d} values of modified aptamers were measured in SB18T0.01 buffer plus 20% glycerol and 1 mM DTT (EMSA) or SB18T0.01 plus 1 mM DTT (filter binding). Modified aptamers were 5′ end labeled using T4 polynucleotide kinase (New England Biolabs) and γ-[³²P]ATP (Perkin-Elmer). Radiolabeled aptamers (20,000-40,000 CPM, ˜0.03 nM) were mixed with varying concentrations of RIG-I proteins, ranging from 10⁻⁹ to 10⁻¹⁴M (EMSA) or 10⁻⁷ to 10⁻¹² M (filter binding) and incubated at 37° C. for 40 minutes.

For EMSA, bound complexes were separated from free DNA on a 10% TBE gel run at 120 V for 45 minutes in 0.5× TBE buffer (44.5 mM Tris base, 44.5 mM Boric acid, 1 mM Ethylenediaminetetraacetic acid (EDTA)). Gels were exposed to a phosphorimaging screen overnight at −20° C. and the fraction of bound aptamer was quantified with a phosphorimager (Typhoon FLA 9500, GE) and data were analyzed in ImageQuant (GE).

For filter binding, bound complexes were partitioned on Zorbax beads (Agilent) and captured on Durapore filter plates (EMD Millipore) and the fraction of bound aptamer was quantified with a phosphorimager (Typhoon FLA 9500, GE) and data were analyzed in ImageQuant (GE).

To determine binding affinity, data were fit using the equation:

y=(max−min)(Protein)/(K_{d}+Protein)+min. and plotted using GraphPad Prism version 7.00.

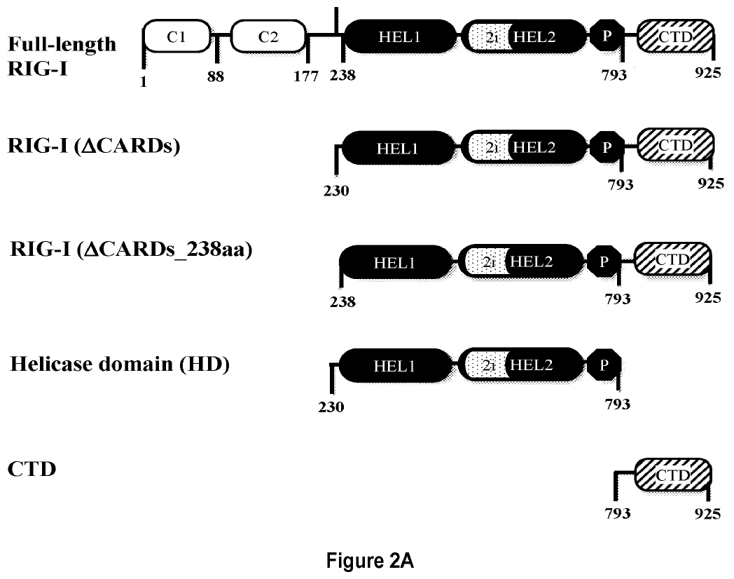

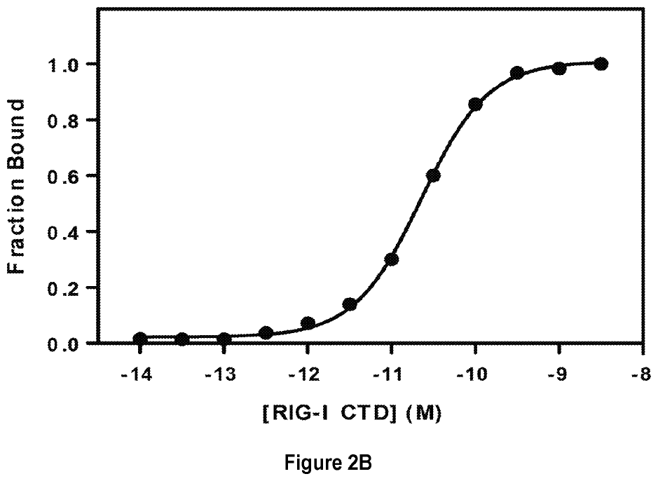

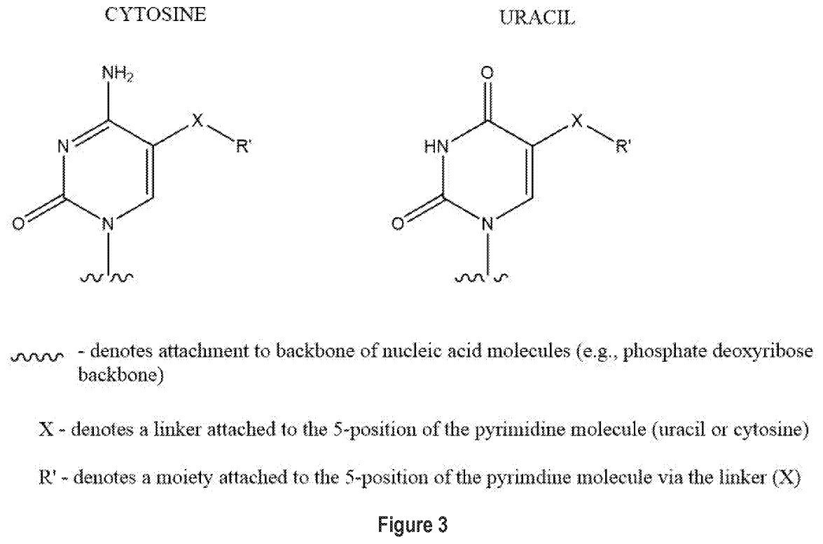

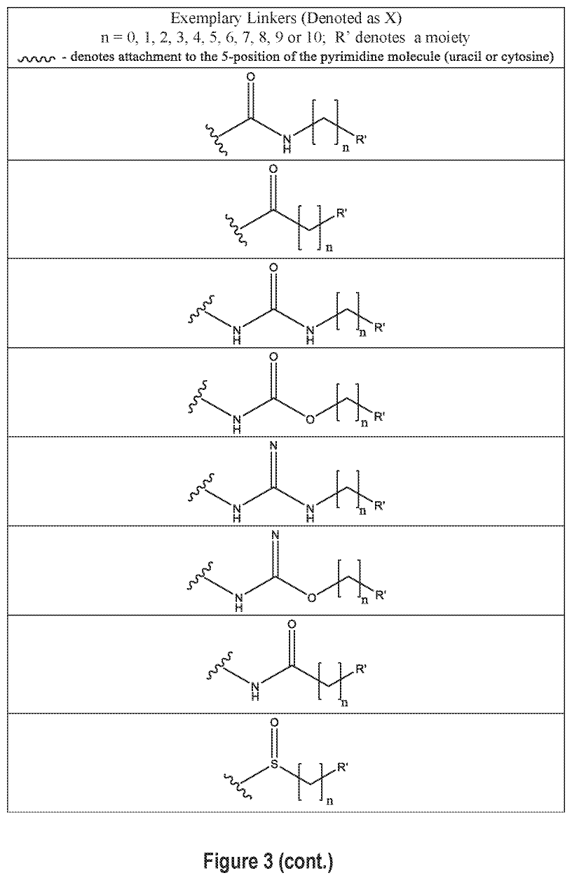

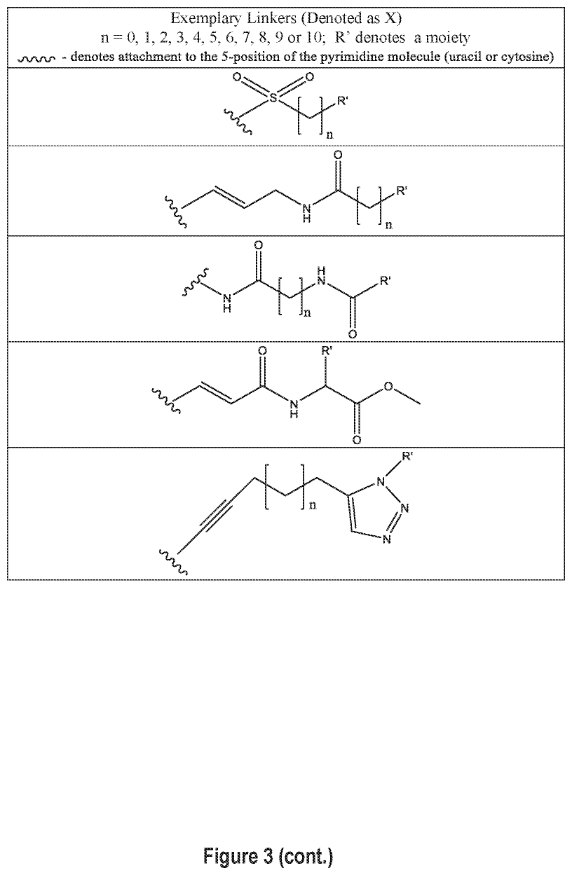

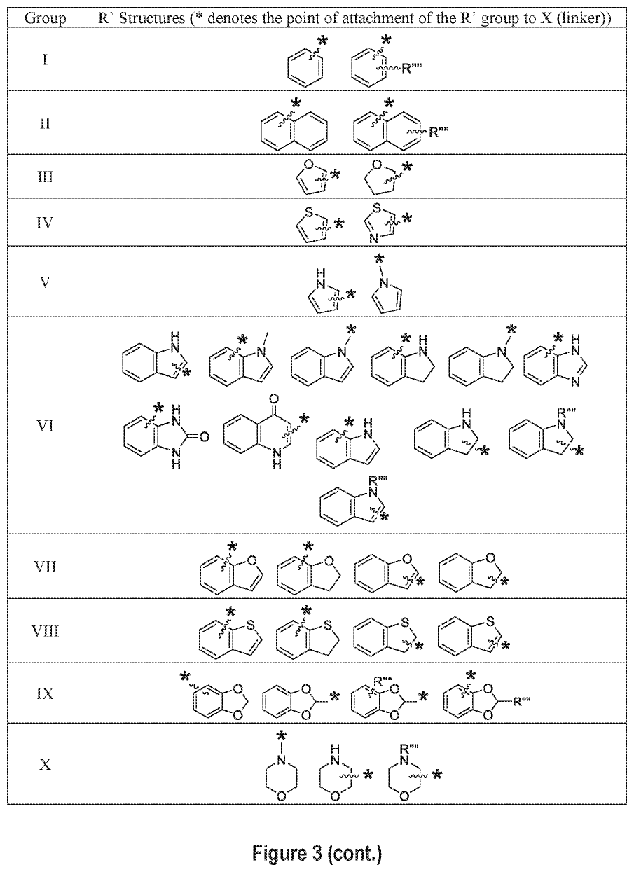

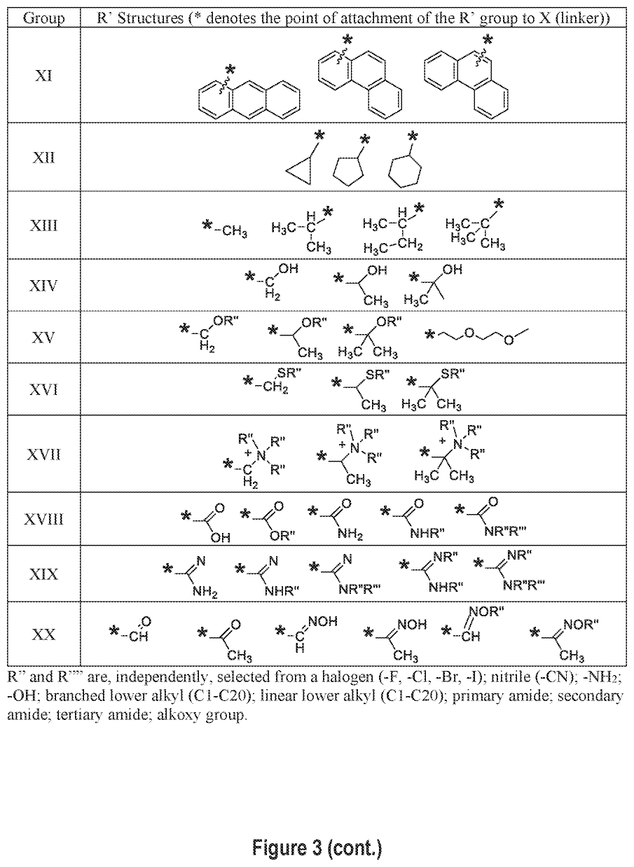

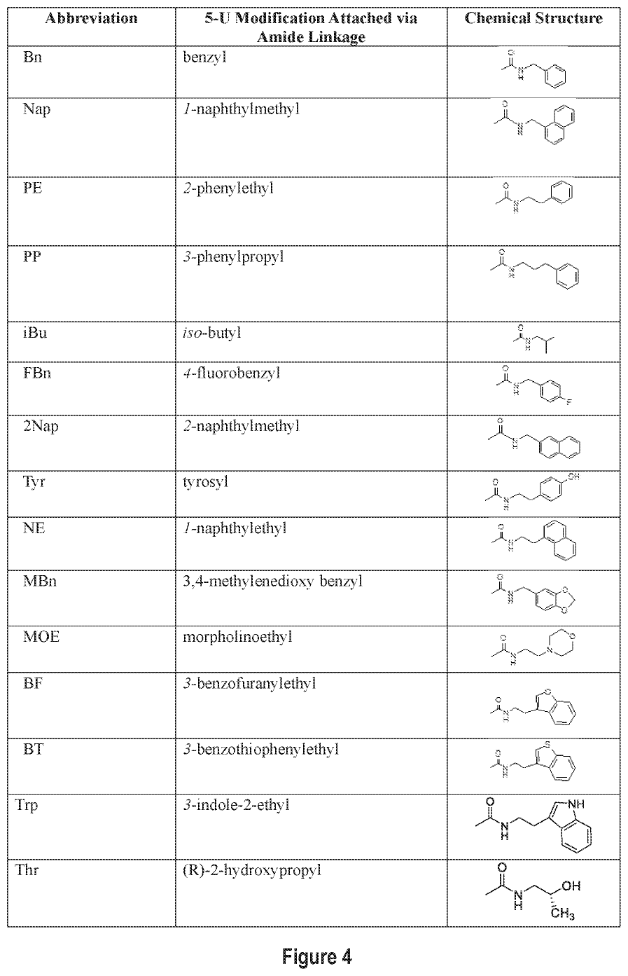

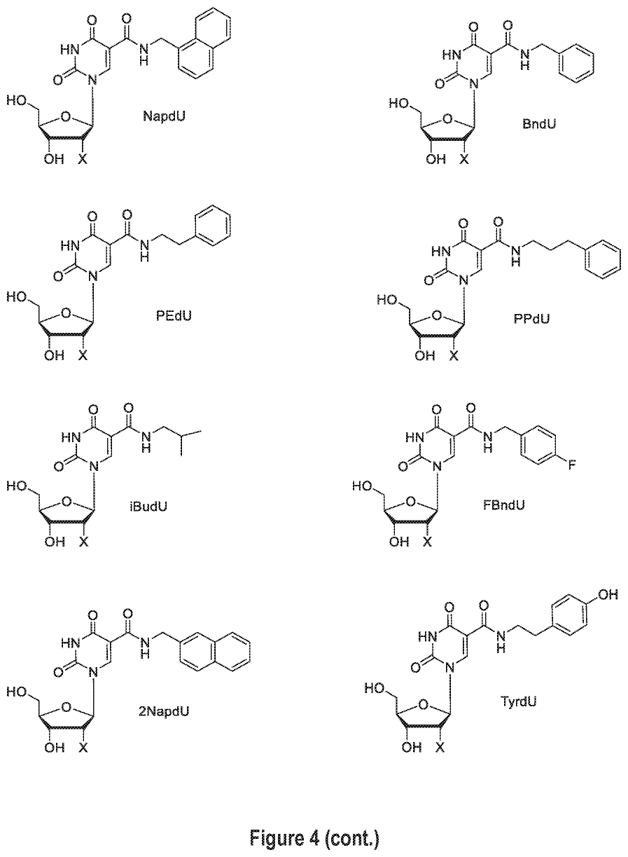

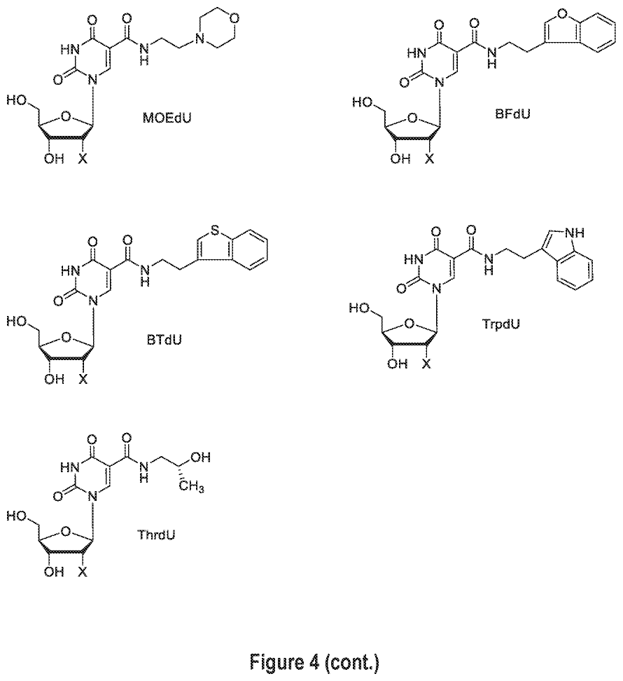

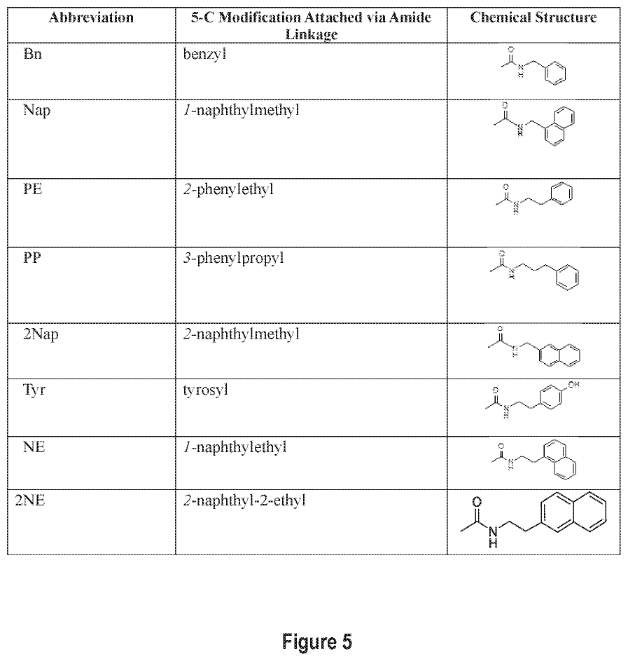

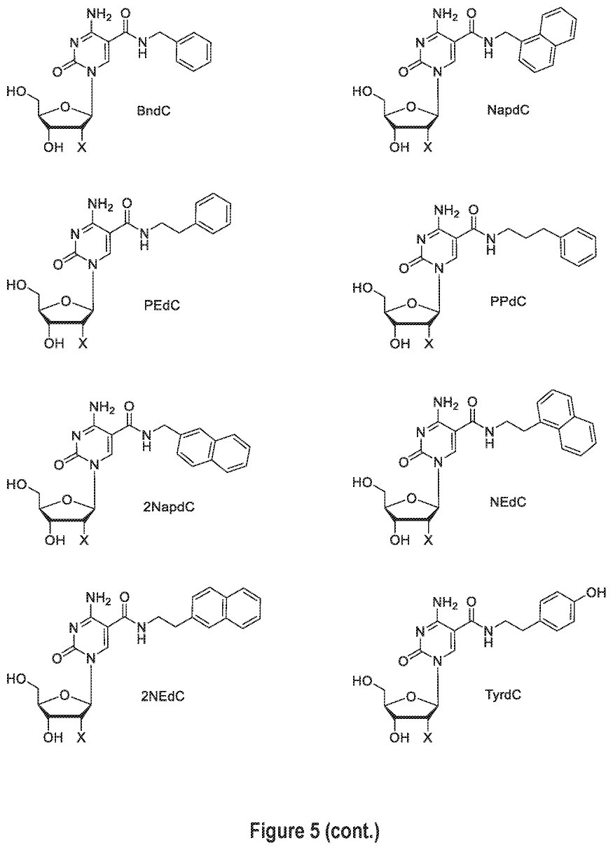

Binding results are shown in Table 5. The amino acid sequences of the RIG-I proteins used in the binding assays are shown in Table 6, and a diagram of certain RIG-I proteins from Vela et al. The Thermodynamic Basis for Viral RNA Detection by the RIG-I Innate Immune Sensor. J. Biol. Chem. 287(51): 42564, 2012 is shown in FIG. 2A.

TABLE 4

| | Sequence (5′ to 3′), | SEQ |
|---|---|---|
| Aptamer | “J” is 5-(N-3- | ID |
| ID | phenylpropylcarboxyamide) uracil | NO. |
| 14832-55_3 | ATCTCJGJGJCAJGJGJJJJAAJGJCAGJJJA | 4 |
| | AJGAJGAGGGACGGGCAG | |
| 14832-55_4 | TCJGJGJCAJGJGJJJJAAJGJCAGJJJAAJG | 5 |
| | AJGAGGGACGGGCAG | |
| 14832-55_5 | GJGJCAJGJGJJJJAAJGJCAGJJJAAJGAJG | 6 |
| | AGGGACGGGCAG | |
| 14832-55_6 | ATCTCJGJGJCAJGJGJJJJAAJGJCAGJJJA | 7 |
| | AJGAJGAGGGACGGG | |
| 14832-55_7 | ATCTCJGJGJCAJGJGJJJJAAJGJCAGJJJA | 8 |
| | AJGAJGAGGGAC | |
| 14832-55_8 | ATCTCJGJGJCAJGJGJJJJAAJGJCAGJJJA | 9 |
| | AJGAJGAGG | |
| 14832-55_9 | ATCTCJGJGJCAJGJGJJJJAAJGJCAGJJJA | 10 |
| | AJGAJG | |
| 14832-55_10 | ATCTCJGJGJCAJGJGJJJJAAJGJCAGJJJA | 11 |
| | AJG | |
| 14832-55_11 | TCJGJGJCAJGJGJJJJAAJGJCAGJJJAAJG | 12 |
| | AJGAGG | |
| 14832-55_12 | CJGJGJCAJGJGJJJJAAJGJCAGJJJAAJGA | 13 |
| | JGAGG | |
| 14832-55_13 | JGJGJCAJGJGJJJJAAJGJCAGJJJAAJGAJ | 14 |
| | GAGG | |
| 14832-55_14 | TCJGJGJCAJGJGJJJJAAJGJCAGJJJAAJG | 15 |
| | AJGAG | |
| 14832-55_15 | TCJGJGJCAJGJGJJJJAAJGJCAGJJJAAJG | 16 |
| | AJGA | |

TABLE 5

| Aptamer ID | Protein | K_{d} (M) |
|---|---|---|
| 14832-55_3 | RIG-I CTD | 7.2E−12 |
| 14832-55_3 | WT full-length RIG-I | 2.4E−11 |
| 14832-55_4 | RIG-I CTD | 8.4E−11 |
| 14832-55_5 | RIG-I CTD | 3.6E−08 |
| 14832-55_6 | RIG-I CTD | 1.8E−11 |
| 14832-55_7 | RIG-I CTD | 1.1E−11 |
| 14832-55_8 | RIG-I CTD | 1.9E−11 |
| 14832-55_8 | RIG-I helicase domain | 1.0E−08 |
| 14832-55_8 | RIG-I ΔCARDs | 1.3E−11 |
| 14832-55_8 | WT full-length RIG-I | 5.6E−10 |
| 14832-55_8 | RIG-I D872A | 6.1E−10 |
| 14832-55_8 | RIG-I K888A | 5.3E−10 |
| 14832-55_8 | RIG-I K861A | 8.9E−10 |
| 14832-55_8 | RIG-I K858A | 2.9E−09 |
| 14832-55_8 | RIG-I H847A | 9.9E−09 |
| 14832-55_8 | RIG-I N668A | 1.1E−10 |
| 14832-55_8 | RIG-I K849A | 6.2E−10 |
| 14832-55_8 | RIG-I R664A | 4.1E−10 |
| 14832-55_9 | RIG-I CTD | 1.5E−10 |
| 14832-55_10 | RIG-I CTD | 1.5E−09 |
| 14832-55_11 | RIG-I CTD | 6.8E−11 |
| 14832-55_12 | RIG-I CTD | 3.4E−10 |
| 14832-55_13 | RIG-I CTD | 4.3E−09 |
| 14832-55_14 | RIG-I CTD | 6.6E−11 |
| 14832-55_15 | RIG-I CTD | 6.3E−11 |
| 14833-10_3 | WT full-length RIG-I | 1.2E−08 |

TABLE 6

| | | SEQ |
|---|---|---|
| Protein | Sequence | ID NO. |
| Wild type | MTTEQRRSLQ AFQDYIRKTL DPTYILSYMA PWFREEEVQY IQAEKNNKGP | 53 |
| (WT) full- | MEAATLFLKF LLELQEEGWF RGFLDALDHA GYSGLYEAIE SWDFKKIEKL | |
| length | EEYRLLLKRL QPEFKTRIIP TDIISDLSEC LINQECEEIL QICSTKGMMA | |
| RIG-I (aa | GAEKLVECLL RSDKENWPKT LKLALEKERN KFSELWIVEK GIKDVETEDL | |
| 1-925) | EDKMETSDIQ IFYQEDPECQ NLSENSCPPS EVSDTNLYSP FKPRNYQLEL | |
| | ALPAMKGKNT IICAPTGCGK TFVSLLICEH HLKKFPQGQK GKVVFFANQI | |
| | PVYEQQKSVF SKYFERHGYR VTGISGATAE NVPVEQIVEN NDIIILTPQI | |
| | LVNNLKKGTI PSLSIFTLMI FDECHNTSKQ HPYNMIMFNY LDQKLGGSSG | |
| | PLPQVIGLTA SVGVGDAKNT DEALDYICKL CASLDASVIA TVKHNLEELE | |
| | QVVYKPQKFF RKVESRISDK FKYIIAQLMR DTESLAKRIC KDLENLSQIQ | |
| | NREFGTQKYE QWIVTVQKAC MVFQMPDKDE ESRICKALFL YTSHLRKYND | |
| | ALIISEHARM KDALDYLKDF FSNVRAAGFD EIEQDLTQRF EEKLQELESV | |
| | SRDPSNENPK LEDLCFILQE EYHLNPETIT ILFVKTRALV DALKNWIEGN | |
| | PKLSFLKPGI LTGRGKTNQN TGMTLPAQKC ILDAFKASGD HNILIATSVA | |
| | DEGIDIAQCN LVILYEYVGN VIKMIQTRGR GRARGSKCFL LTSNAGVIEK | |
| | EQINMYKEKM MNDSILRLQT WDEAVFREKI LHIQTHEKFI RDSQEKPKPV | |
| | PDKENKKLLC RKCKALACYT ADVRVIEECH YTVLGDAFKE CFVSRPHPKP | |
| | KQFSSFEKRA KIFCARQNCS HDWGIHVKYK TFEIPVIKIE SFVVEDIATG | |
| | VQTLYSKWKD FHFEKIPFDP AEMSK | |
| RIG-I | PS EVSDTNLYSP FKPRNYQLELALPAMKGKNT IICAPTGCGK TFVSLLICEH | 54 |
| ΔCARDS | HLKKFPQGQK GKVVFFANQIPVYEQQKSVF SKYFERHGYR VTGISGATAE | |
| (aa 229- | NVPVEQIVEN NDIIILTPQILVNNLKKGTI PSLSIFTLMI FDECHNTSKQ | |
| 925) | HPYNMIMFNY LDQKLGGSSGPLPQVIGLTA SVGVGDAKNT DEALDYICKL | |
| | CASLDASVIA TVKHNLEELEQVVYKPQKFF RKVESRISDK FKYIIAQLMR | |
| | DTESLAKRIC KDLENLSQIQNREFGTQKYE QWIVTVQKAC MVFQMPDKDE | |
| | ESRICKALFL YTSHLRKYNDALIISEHARM KDALDYLKDF FSNVRAAGFD | |
| | EIEQDLTQRF EEKLQELESVSRDPSNENPK LEDLCFILQE EYHLNPETIT | |
| | ILFVKTRALV DALKNWIEGNPKLSFLKPGI LTGRGKTNQN TGMTLPAQKC | |
| | ILDAFKASGD HNILIATSVADEGIDIAQCN LVILYEYVGN VIKMIQTRGR | |
| | GRARGSKCFL LTSNAGVIEKEQINMYKEKM MNDSILRLQT WDEAVFREKI | |
| | LHIQTHEKFI RDSQEKPKPVPDKENKKLLC RKCKALACYT ADVRVIEECH | |
| | YTVLGDAFKE CFVSRPHPKPKQFSSFEKRA KIFCARQNCS HDWGIHVKYK | |
| | TFEIPVIKIE SFVVEDIATGVQTLYSKWKD FHFEKIPFDP AEMSK | |
| RIG-I | PS EVSDTNLYSP FKPRNYQLELALPAMKGKNT IICAPTGCGK TFVSLLICEH | 55 |
| Helicase | HLKKFPQGQK GKVVFFANQIPVYEQQKSVF SKYFERHGYR VTGISGATAE | |
| Domain | NVPVEQIVEN NDIIILTPQILVNNLKKGTI PSLSIFTLMI FDECHNTSKQ | |
| (aa 229- | HPYNMIMFNY LDQKLGGSSGPLPQVIGLTA SVGVGDAKNT DEALDYICKL | |
| 793) | CASLDASVIA TVKHNLEELEQVVYKPQKFF RKVESRISDK FKYIIAQLMR | |
| | DTESLAKRIC KDLENLSQIQNREFGTQKYE QWIVTVQKAC MVFQMPDKDE | |
| | ESRICKALFL YTSHLRKYNDALIISEHARM KDALDYLKDF FSNVRAAGFD | |
| | EIEQDLTQRF EEKLQELESVSRDPSNENPK LEDLCFILQE EYHLNPETIT | |
| | ILFVKTRALV DALKNWIEGNPKLSFLKPGI LTGRGKTNQN TGMTLPAQKC | |
| | ILDAFKASGD HNILIATSVADEGIDIAQCN LVILYEYVGN VIKMIQTRGR | |
| | GRARGSKCFL LTSNAGVIEKEQINMYKEKM MNDSILRLQT WDEAVFREKI | |
| | LHIQTHEKFI RDS | |
| RIG-I | DSQEKPKPVPDKENKKLLC RKCKALACYT ADVRVIEECH YTVLGDAFKE | 56 |
| CTD (aa | CFVSRPHPKPKQFSSFEKRA KIFCARQNCS HDWGIHVKYK TFEIPVIKIE | |
| 792-925) | SFVVEDIATGVQTLYSKWKD FHFEKIPFDP AEMSK | |
| RIG-I | MTTEQRRSLQ AFQDYIRKTL DPTYILSYMA PWFREEEVQY IQAEKNNKGP | 57 |
| D872A | MEAATLFLKF LLELQEEGWF RGFLDALDHA GYSGLYEAIE SWDFKKIEKL | |
| | EEYRLLLKRL QPEFKTRIIP TDIISDLSEC LINQECEEIL QICSTKGMMA | |
| | GAEKLVECLL RSDKENWPKT LKLALEKERN KFSELWIVEK GIKDVETEDL | |
| | EDKMETSDIQ IFYQEDPECQ NLSENSCPPS EVSDTNLYSP FKPRNYQLEL | |
| | ALPAMKGKNT IICAPTGCGK TFVSLLICEH HLKKFPQGQK GKVVFFANQI | |
| | PVYEQQKSVF SKYFERHGYR VTGISGATAE NVPVEQIVEN NDIIILTPQI | |
| | LVNNLKKGTI PSLSIFTLMI FDECHNTSKQ HPYNMIMFNY LDQKLGGSSG | |
| | PLPQVIGLTA SVGVGDAKNT DEALDYICKL CASLDASVIA TVKHNLEELE | |
| | QVVYKPQKFF RKVESRISDK FKYIIAQLMR DTESLAKRIC KDLENLSQIQ | |
| | NREFGTQKYE QWIVTVQKAC MVFQMPDKDE ESRICKALFL YTSHLRKYND | |
| | ALIISEHARM KDALDYLKDF FSNVRAAGFD EIEQDLTQRF EEKLQELESV | |
| | SRDPSNENPK LEDLCFILQE EYHLNPETIT ILFVKTRALV DALKNWIEGN | |
| | PKLSFLKPGI LTGRGKTNQN TGMTLPAQKC ILDAFKASGD HNILIATSVA | |
| | DEGIDIAQCN LVILYEYVGN VIKMIQTRGR GRARGSKCFL LTSNAGVIEK | |
| | EQINMYKEKM MNDSILRLQT WDEAVFREKI LHIQTHEKFI RDSQEKPKPV | |
| | PDKENKKLLC RKCKALACYT ADVRVIEECH YTVLGDAFKE CFVSRPHPKP | |
| | KQFSSFEKRA KIFCARQNCS HAWGIHVKYK TFEIPVIKIE SFVVEDIATG | |
| | VQTLYSKWKD FHFEKIPFDP AEMSK | |
| RIG-I | MTTEQRRSLQ AFQDYIRKTL DPTYILSYMA PWFREEEVQY IQAEKNNKGP | 58 |
| N668A | MEAATLFLKF LLELQEEGWF RGFLDALDHA GYSGLYEAIE SWDFKKIEKL | |
| | EEYRLLLKRL QPEFKTRIIP TDIISDLSEC LINQECEEIL QICSTKGMMA | |
| | GAEKLVECLL RSDKENWPKT LKLALEKERN KFSELWIVEK GIKDVETEDL | |
| | EDKMETSDIQ IFYQEDPECQ NLSENSCPPS EVSDTNLYSP FKPRNYQLEL | |
| | ALPAMKGKNT IICAPTGCGK TFVSLLICEH HLKKFPQGQK GKVVFFANQI | |
| | PVYEQQKSVF SKYFERHGYR VTGISGATAE NVPVEQIVEN NDIIILTPQI | |
| | LVNNLKKGTI PSLSIFTLMI FDECHNTSKQ HPYNMIMFNY LDQKLGGSSG | |
| | PLPQVIGLTA SVGVGDAKNT DEALDYICKL CASLDASVIA TVKHNLEELE | |
| | QVVYKPQKFF RKVESRISDK FKYIIAQLMR DTESLAKRIC KDLENLSQIQ | |
| | NREFGTQKYE QWIVTVQKAC MVFQMPDKDE ESRICKALFL YTSHLRKYND | |
| | ALIISEHARM KDALDYLKDF FSNVRAAGFD EIEQDLTQRF EEKLQELESV | |
| | SRDPSNENPK LEDLCFILQE EYHLNPETIT ILFVKTRALV DALKNWIEGN | |
| | PKLSFLKPGI LTGRGKTAQN TGMTLPAQKC ILDAFKASGD HNILIATSVA | |
| | DEGIDIAQCN LVILYEYVGN VIKMIQTRGR GRARGSKCFL LTSNAGVIEK | |
| | EQINMYKEKM MNDSILRLQT WDEAVFREKI LHIQTHEKFI RDSQEKPKPV | |
| | PDKENKKLLC RKCKALACYT ADVRVIEECH YTVLGDAFKE CFVSRPHPKP | |
| | KQFSSFEKRA KIFCARQNCS HDWGIHVKYK TFEIPVIKIE SFVVEDIATG | |
| | VQTLYSKWKD FHFEKIPFDP AEMSK | |
| RIG-I | MTTEQRRSLQ AFQDYIRKTL DPTYILSYMA PWFREEEVQY IQAEKNNKGP | 59 |
| K849A | MEAATLFLKF LLELQEEGWF RGFLDALDHA GYSGLYEAIE SWDFKKIEKL | |
| | EEYRLLLKRL QPEFKTRIIP TDIISDLSEC LINQECEEIL QICSTKGMMA | |
| | GAEKLVECLL RSDKENWPKT LKLALEKERN KFSELWIVEK GIKDVETEDL | |
| | EDKMETSDIQ IFYQEDPECQ NLSENSCPPS EVSDTNLYSP FKPRNYQLEL | |
| | ALPAMKGKNT IICAPTGCGK TFVSLLICEH HLKKFPQGQK GKVVFFANQI | |
| | PVYEQQKSVF SKYFERHGYR VTGISGATAE NVPVEQIVEN NDIIILTPQI | |
| | LVNNLKKGTI PSLSIFTLMI FDECHNTSKQ HPYNMIMFNY LDQKLGGSSG | |
| | PLPQVIGLTA SVGVGDAKNT DEALDYICKL CASLDASVIA TVKHNLEELE | |
| | QVVYKPQKFF RKVESRISDK FKYIIAQLMR DTESLAKRIC KDLENLSQIQ | |
| | NREFGTQKYE QWIVTVQKAC MVFQMPDKDE ESRICKALFL YTSHLRKYND | |
| | ALIISEHARM KDALDYLKDF FSNVRAAGFD EIEQDLTQRF EEKLQELESV | |
| | SRDPSNENPK LEDLCFILQE EYHLNPETIT ILFVKTRALV DALKNWIEGN | |
| | PKLSFLKPGI LTGRGKTNQN TGMTLPAQKC ILDAFKASGD HNILIATSVA | |
| | DEGIDIAQCN LVILYEYVGN VIKMIQTRGR GRARGSKCFL LTSNAGVIEK | |
| | EQINMYKEKM MNDSILRLQT WDEAVFREKI LHIQTHEKFI RDSQEKPKPV | |
| | PDKENKKLLC RKCKALACYT ADVRVIEECH YTVLGDAFKE CFVSRPHPAP | |
| | KQFSSFEKRA KIFCARQNCS HDWGIHVKYK TFEIPVIKIE SFVVEDIATG | |
| | VQTLYSKWKD FHFEKIPFDP AEMSK | |
| RIG-I | MTTEQRRSLQ AFQDYIRKTL DPTYILSYMA PWFREEEVQY IQAEKNNKGP | 60 |
| K888A | MEAATLFLKF LLELQEEGWF RGFLDALDHA GYSGLYEAIE SWDFKKIEKL | |
| | EEYRLLLKRL QPEFKTRIIP TDIISDLSEC LINQECEEIL QICSTKGMMA | |
| | GAEKLVECLL RSDKENWPKT LKLALEKERN KFSELWIVEK GIKDVETEDL | |
| | EDKMETSDIQ IFYQEDPECQ NLSENSCPPS EVSDTNLYSP FKPRNYQLEL | |
| | ALPAMKGKNT IICAPTGCGK TFVSLLICEH HLKKFPQGQK GKVVFFANQI | |
| | PVYEQQKSVF SKYFERHGYR VTGISGATAE NVPVEQIVEN NDIIILTPQI | |
| | LVNNLKKGTI PSLSIFTLMI FDECHNTSKQ HPYNMIMFNY LDQKLGGSSG | |
| | PLPQVIGLTA SVGVGDAKNT DEALDYICKL CASLDASVIA TVKHNLEELE | |
| | QVVYKPQKFF RKVESRISDK FKYIIAQLMR DTESLAKRIC KDLENLSQIQ | |
| | NREFGTQKYE QWIVTVQKAC MVFQMPDKDE ESRICKALFL YTSHLRKYND | |
| | ALIISEHARM KDALDYLKDF FSNVRAAGFD EIEQDLTQRF EEKLQELESV | |
| | SRDPSNENPK LEDLCFILQE EYHLNPETIT ILFVKTRALV DALKNWIEGN | |
| | PKLSFLKPGI LTGRGKTNQN TGMTLPAQKC ILDAFKASGD HNILIATSVA | |
| | DEGIDIAQCN LVILYEYVGN VIKMIQTRGR GRARGSKCFL LTSNAGVIEK | |
| | EQINMYKEKM MNDSILRLQT WDEAVFREKI LHIQTHEKFI RDSQEKPKPV | |
| | PDKENKKLLC RKCKALACYT ADVRVIEECH YTVLGDAFKE CFVSRPHPKP | |
| | KQFSSFEKRA KIFCARQNCS HDWGIHVKYK TFEIPVIAIE SFVVEDIATG | |
| | VQTLYSKWKD FHFEKIPFDP AEMSK | |
| RIG-I | MTTEQRRSLQ AFQDYIRKTL DPTYILSYMA PWFREEEVQY IQAEKNNKGP | 61 |
| R664A | MEAATLFLKF LLELQEEGWF RGFLDALDHA GYSGLYEAIE SWDFKKIEKL | |
| | EEYRLLLKRL QPEFKTRIIP TDIISDLSEC LINQECEEIL QICSTKGMMA | |
| | GAEKLVECLL RSDKENWPKT LKLALEKERN KFSELWIVEK GIKDVETEDL | |
| | EDKMETSDIQ IFYQEDPECQ NLSENSCPPS EVSDTNLYSP FKPRNYQLEL | |
| | ALPAMKGKNT IICAPTGCGK TFVSLLICEH HLKKFPQGQK GKVVFFANQI | |
| | PVYEQQKSVF SKYFERHGYR VTGISGATAE NVPVEQIVEN NDIIILTPQI | |
| | LVNNLKKGTI PSLSIFTLMI FDECHNTSKQ HPYNMIMFNY LDQKLGGSSG | |
| | PLPQVIGLTA SVGVGDAKNT DEALDYICKL CASLDASVIA TVKHNLEELE | |
| | QVVYKPQKFF RKVESRISDK FKYIIAQLMR DTESLAKRIC KDLENLSQIQ | |
| | NREFGTQKYE QWIVTVQKAC MVFQMPDKDE ESRICKALFL YTSHLRKYND | |
| | ALIISEHARM KDALDYLKDF FSNVRAAGFD EIEQDLTQRF EEKLQELESV | |
| | SRDPSNENPK LEDLCFILQE EYHLNPETIT ILFVKTRALV DALKNWIEGN | |
| | PKLSFLKPGI LTGAGKTNQN TGMTLPAQKC ILDAFKASGD HNILIATSVA | |
| | DEGIDIAQCN LVILYEYVGN VIKMIQTRGR GRARGSKCFL LTSNAGVIEK | |
| | EQINMYKEKM MNDSILRLQT WDEAVFREKI LHIQTHEKFI RDSQEKPKPV | |
| | PDKENKKLLC RKCKALACYT ADVRVIEECH YTVLGDAFKE CFVSRPHPKP | |
| | KQFSSFEKRA KIFCARQNCS HDWGIHVKYK TFEIPVIKIE SFVVEDIATG | |
| | VQTLYSKWKD FHFEKIPFDP AEMSK | |
| RIG-I | MTTEQRRSLQ AFQDYIRKTL DPTYILSYMA PWFREEEVQY IQAEKNNKGP | 62 |
| H847A | MEAATLFLKF LLELQEEGWF RGFLDALDHA GYSGLYEAIE SWDFKKIEKL | |
| | EEYRLLLKRL QPEFKTRIIP TDIISDLSEC LINQECEEIL QICSTKGMMA | |
| | GAEKLVECLL RSDKENWPKT LKLALEKERN KFSELWIVEK GIKDVETEDL | |
| | EDKMETSDIQ IFYQEDPECQ NLSENSCPPS EVSDTNLYSP FKPRNYQLEL | |
| | ALPAMKGKNT IICAPTGCGK TFVSLLICEH HLKKFPQGQK GKVVFFANQI | |
| | PVYEQQKSVF SKYFERHGYR VTGISGATAE NVPVEQIVEN NDIIILTPQI | |
| | LVNNLKKGTI PSLSIFTLMI FDECHNTSKQ HPYNMIMFNY LDQKLGGSSG | |
| | PLPQVIGLTA SVGVGDAKNT DEALDYICKL CASLDASVIA TVKHNLEELE | |
| | QVVYKPQKFF RKVESRISDK FKYIIAQLMR DTESLAKRIC KDLENLSQIQ | |
| | NREFGTQKYE QWIVTVQKAC MVFQMPDKDE ESRICKALFL YTSHLRKYND | |
| | ALIISEHARM KDALDYLKDF FSNVRAAGFD EIEQDLTQRF EEKLQELESV | |
| | SRDPSNENPK LEDLCFILQE EYHLNPETIT ILFVKTRALV DALKNWIEGN | |
| | PKLSFLKPGI LTGRGKTNQN TGMTLPAQKC ILDAFKASGD HNILIATSVA | |
| | DEGIDIAQCN LVILYEYVGN VIKMIQTRGR GRARGSKCFL LTSNAGVIEK | |
| | EQINMYKEKM MNDSILRLQT WDEAVFREKI LHIQTHEKFI RDSQEKPKPV | |
| | PDKENKKLLC RKCKALACYT ADVRVIEECH YTVLGDAFKE CFVSRPAPKP | |
| | KQFSSFEKRA KIFCARQNCS HDWGIHVKYK TFEIPVIKIE SFVVEDIATG | |
| | VQTLYSKWKD FHFEKIPFDP AEMSK | |
| RIG-I | MTTEQRRSLQ AFQDYIRKTL DPTYILSYMA PWFREEEVQY IQAEKNNKGP | 63 |
| K858A | MEAATLFLKF LLELQEEGWF RGFLDALDHA GYSGLYEAIE SWDFKKIEKL | |
| | EEYRLLLKRL QPEFKTRIIP TDIISDLSEC LINQECEEIL QICSTKGMMA | |
| | GAEKLVECLL RSDKENWPKT LKLALEKERN KFSELWIVEK GIKDVETEDL | |
| | EDKMETSDIQ IFYQEDPECQ NLSENSCPPS EVSDTNLYSP FKPRNYQLEL | |
| | ALPAMKGKNT IICAPTGCGK TFVSLLICEH HLKKFPQGQK GKVVFFANQI | |
| | PVYEQQKSVF SKYFERHGYR VTGISGATAE NVPVEQIVEN NDIIILTPQI | |
| | LVNNLKKGTI PSLSIFTLMI FDECHNTSKQ HPYNMIMFNY LDQKLGGSSG | |
| | PLPQVIGLTA SVGVGDAKNT DEALDYICKL CASLDASVIA TVKHNLEELE | |
| | QVVYKPQKFF RKVESRISDK FKYIIAQLMR DTESLAKRIC KDLENLSQIQ | |
| | NREFGTQKYE QWIVTVQKAC MVFQMPDKDE ESRICKALFL YTSHLRKYND | |
| | ALIISEHARM KDALDYLKDF FSNVRAAGFD EIEQDLTQRF EEKLQELESV | |
| | SRDPSNENPK LEDLCFILQE EYHLNPETIT ILFVKTRALV DALKNWIEGN | |
| | PKLSFLKPGI LTGRGKTNQN TGMTLPAQKC ILDAFKASGD HNILIATSVA | |
| | DEGIDIAQCN LVILYEYVGN VIKMIQTRGR GRARGSKCFL LTSNAGVIEK | |
| | EQINMYKEKM MNDSILRLQT WDEAVFREKI LHIQTHEKFI RDSQEKPKPV | |
| | PDKENKKLLC RKCKALACYT ADVRVIEECH YTVLGDAFKE CFVSRPHPKP | |
| | KQFSSFEARA KIFCARQNCS HDWGIHVKYK TFEIPVIKIE SFVVEDIATG | |
| | VQTLYSKWKD FHFEKIPFDP AEMSK | |
| RIG-I | MTTEQRRSLQ AFQDYIRKTL DPTYILSYMA PWFREEEVQY IQAEKNNKGP | 64 |
| K861A | MEAATLFLKF LLELQEEGWF RGFLDALDHA GYSGLYEAIE SWDFKKIEKL | |
| | EEYRLLLKRL QPEFKTRIIP TDIISDLSEC LINQECEEIL QICSTKGMMA | |
| | GAEKLVECLL RSDKENWPKT LKLALEKERN KFSELWIVEK GIKDVETEDL | |
| | EDKMETSDIQ IFYQEDPECQ NLSENSCPPS EVSDTNLYSP FKPRNYQLEL | |
| | ALPAMKGKNT IICAPTGCGK TFVSLLICEH HLKKFPQGQK GKVVFFANQI | |
| | PVYEQQKSVF SKYFERHGYR VTGISGATAE NVPVEQIVEN NDIIILTPQI | |
| | LVNNLKKGTI PSLSIFTLMI FDECHNTSKQ HPYNMIMFNY LDQKLGGSSG | |
| | PLPQVIGLTA SVGVGDAKNT DEALDYICKL CASLDASVIA TVKHNLEELE | |
| | QVVYKPQKFF RKVESRISDK FKYIIAQLMR DTESLAKRIC KDLENLSQIQ | |
| | NREFGTQKYE QWIVTVQKAC MVFQMPDKDE ESRICKALFL YTSHLRKYND | |
| | ALIISEHARM KDALDYLKDF FSNVRAAGFD EIEQDLTQRF EEKLQELESV | |
| | SRDPSNENPK LEDLCFILQE EYHLNPETIT ILFVKTRALV DALKNWIEGN | |
| | PKLSFLKPGI LTGRGKTNQN TGMTLPAQKC ILDAFKASGD HNILIATSVA | |
| | DEGIDIAQCN LVILYEYVGN VIKMIQTRGR GRARGSKCFL LTSNAGVIEK | |
| | EQINMYKEKM MNDSILRLQT WDEAVFREKI LHIQTHEKFI RDSQEKPKPV | |
| | PDKENKKLLC RKCKALACYT ADVRVIEECH YTVLGDAFKE CFVSRPHPKP | |
| | KQFSSFEKRA AIFCARQNCS HDWGIHVKYK TFEIPVIKIE SFVVEDIATG | |
| | VQTLYSKWKD FHFEKIPFDP AEMSK | |

1. An aptamer that binds RIG-I protein, wherein the aptamer comprises the sequence 5′-PEPSZV-3′ (SEQ ID NO: 49)
wherein,
P is independently, and for each occurrence, a C-5 modified pyrimidine;
E is a C-5 modified pyrimidine, A or G;
S is a G or C;
Z is a C-5 modified pyrimidine or A;
V is a C, A, or G; and
wherein the aptamer is at least 30, 40, 50, 60, 70, 80, 90, or 100 nucleotides in length.

2. An aptamer that binds RIG-I protein, wherein the aptamer comprises the sequence 5′-PEPSFP-3′ (SEQ ID NO: 50)
wherein,
P is independently, and for each occurrence, a C-5 modified pyrimidine;
E is a C-5 modified pyrimidine, A or G;
S is a G or C;
F is a C-5 modified pyrimidine, unmodified C, G, or A; and
wherein the aptamer is at least 30, 40, 50, 60, 70, 80, 90, or 100 nucleotides in length.

3. An aptamer that binds RIG-I protein, wherein the aptamer comprises the sequence 5′-PEPSZV-3′ (SEQ ID NO: 49) and the sequence 5′- PEPSFP -3′ (SEQ ID NO: 50)
wherein,
P is independently, and for each occurrence, a C-5 modified pyrimidine;
E is independently, and for each occurrence, a C-5 modified pyrimidine, A or G;
S is independently, and for each occurrence, a G or C;
Z is a C-5 modified pyrimidine or A;
V is a C, A or G; and
F is a C-5 modified pyrimidine, unmodified C, G or A;
wherein, the aptamer is at least 30, 40, 50, 60, 70, 80, 90, or 100 nucleotides in length.

4. An aptamer that binds RIG-I protein, wherein the aptamer comprises the sequence 5′-PEPSZV-3′ (SEQ ID NO: 49), the sequence 5′- PEPSFP-3′ (SEQ ID NO: 50), and the sequence 5′-AAPGAPGAGG-3′ (SEQ ID NO: 51).
wherein,
P is independently, and for each occurrence, a C-5 modified pyrimidine;
E is a C-5 modified pyrimidine, A or G;
S is a G or C;
Z is a C-5 modified pyrimidine or A;
V is a C, A or G and
F is a C-5 modified pyrimidine, unmodified C, G or A;
wherein, the aptamer is at least 30, 40, 50, 60, 70, 80, 90, or 100 nucleotides in length.

5. An aptamer that binds RIG-I protein, wherein the aptamer comprises the sequence 5′-PGPGPCAₙPGPGPPPZAZQQCₙZMGPPAAPGAPGAGG -3′ (SEQ ID NO: 52)
wherein,
P is independently, and for each occurrence, a C-5 modified pyrimidine;
Z is independently, and for each occurrence, a C-5 modified pyrimidine or A;
Q is independently, and for each occurrence, a C-5 modified pyrimidine or G;
M is a C or A; and
n is independently, and for each occurrence, 0 or 1;
wherein the aptamer is at least 35, 40, 50, 60, 70, 80, 90, or 100 nucleotides in length.

6. The aptamer of any one of the preceding claims, wherein each C-5 modified pyrimidine containing nucleoside is independently selected from:
5-(N-benzylcarboxyamide)-2′-deoxyuridine (BndU), 5-(N-benzylcarboxyamide)-2′-O-methyluridine, 5-(N-benzylcarboxyamide)-2′-fluorouridine, 5-(N-phenethylcarboxyamide)-2′-deoxyuridine (PEdU), 5-(N-thiophenylmethylcarboxyamide)-2′-deoxyuridine (ThdU), 5-(N-isobutylcarboxyamide)-2′-deoxyuridine (iBudU), 5-(N-tyrosylcarboxyamide)-2′-deoxyuridine (TyrdU), 5-(N-3,4-methylenedioxybenzylcarboxyamide)-2′-deoxyuridine (MBndU), 5-(N-4-fluorobenzylcarboxyamide)-2′-deoxyuridine (FBndU), 5-(N-3-phenylpropylcarboxyamide)-2′-deoxyuridine (PPdU), 5-(N-imidizolylethylcarboxyamide)-2′-deoxyuridine (ImdU), 5-(N-isobutylcarboxyamide)-2′-O-methyluridine, 5-(N-isobutylcarboxyamide)-2′-fluorouridine, 5-(N-tryptaminocarboxyamide)-2′-deoxyuridine (TrpdU), 5-(N-R-threoninylcarboxyamide)-2′-deoxyuridine (ThrdU), 5-(N-tryptaminocarboxyamide)-2′-O-methyluridine, 5-(N-tryptaminocarboxyamide)-2′-fluorouridine, 5-(N-[1-(3-trimethylamonium) propyl] carboxyamide)-2′-deoxyuridine chloride, 5-(N-naphthylmethylcarboxyamide)-2′-deoxyuridine (NapdU), 5-(N-naphthylmethylcarboxyamide)-2′-O-methyluridine, 5-(N-naphthylmethylcarboxyamide)-2′-fluorouridine, 5-(N-[1-(2,3-dihydroxypropyl)]carboxyamide)-2′-deoxyuridine), 5-(N-2-naphthylmethylcarboxyamide)-2′-deoxyuridine (2NapdU), 5-(N-2-naphthylmethylcarboxyamide)-2′-O-methyluridine, 5-(N-2-naphthylmethylcarboxyamide)-2′-fluorouridine, 5-(N-1-naphthylethylcarboxyamide)-2′-deoxyuridine (NEdU), 5-(N-1-naphthylethylcarboxyamide)-2′-O-methyluridine, 5-(N-1-naphthylethylcarboxyamide)-2′-fluorouridine, 5-(N-2-naphthylethylcarboxyamide)-2′-deoxyuridine (2NEdU), 5-(N-2-naphthylethylcarboxyamide)-2′-O-methyluridine, 5-(N-2-naphthylethylcarboxyamide)-2′-fluorouridine, 5-(N-3-benzofuranylethylcarboxyamide)-2′-deoxyuridine (BFdU), 5-(N-3-benzofuranylethylcarboxyamide)-2′-O-methyluridine, 5-(N-3-benzofuranylethylcarboxyamide)-2′-fluorouridine, 5-(N-3-benzothiophenylethylcarboxyamide)-2′-deoxyuridine (BTdU), 5-(N-3-benzothiophenylethylcarboxyamide)-2′-O-methyluridine, and 5-(N-3-benzothiophenylethylcarboxyamide)-2′-fluorouridine.

7. The aptamer of any one of claims 1-5, wherein each C-5 modified pyrimidine containing nucleoside is 5-(N-3-phenylpropylcarboxyamide)-2′-deoxyuridine (PPdU).

8. The aptamer of any one of the preceding claims, wherein the aptamer comprises the sequence of SEQ ID NO: 65, wherein each nucleoside represented by J is a 5-(N-3-phenylpropylcarboxyamide)-2′-deoxyuridine.

9. The aptamer of any one of the preceding claims, wherein the aptamer comprises the sequence of SEQ ID NO: 66, wherein each nucleoside represented by J is a 5-(N-3-phenylpropylcarboxyamide)-2′-deoxyuridine.

10. The aptamer of any one of the preceding claims, wherein the aptamer comprises the sequence of SEQ ID NO: 67, wherein each nucleoside represented by J is a 5-(N-3-phenylpropylcarboxyamide)-2′-deoxyuridine.

11. The aptamer of any one of the preceding claims, wherein the aptamer comprises a sequence that is at least 80%, at least 85%, at least 90%, at least 95%, at least 97%, or at least 98% identical to a sequence selected from SEQ ID NOs: 4-47 and 68-99, wherein each nucleoside represented by J is a 5-(N-3-phenylpropylcarboxyamide)-2′-deoxyuridine.

12. The aptamer of any one of the preceding claims, wherein the aptamer comprises a sequence selected from SEQ ID NO: 4-47 and 68-99, wherein each nucleoside represented by J is a 5-(N-3-phenylpropylcarboxyamide)-2′-deoxyuridine.

13. An aptamer that binds RIG-I protein, wherein the aptamer comprises a sequence selected from SEQ ID NOs: 4-47 and 68-99, wherein each nucleoside represented by J is a 5-(N-3-phenylpropylcarboxyamide)-2′-deoxyuridine.

14. The aptamer of any one of the preceding claims, wherein the aptamer is from 35 to 60 nucleotides in length, or from 35 to 50 nucleotides in length, or from 40 to 50 nucleotides in length.

15. The aptamer of any one of the preceding claims, wherein the RIG-I protein is a human RIG-I protein.

16. A composition comprising the aptamer of any one of the preceding claims and a RIG-I protein.

17. The composition of claim 16, wherein the RIG-I protein is a human RIG-I protein.

18. A method for selecting an aptamer having binding affinity for a RIG-I protein comprising:
(a) contacting a candidate mixture with a RIG-I protein, wherein the candidate mixture comprises modified nucleic acids in which one, several or all pyrimidines in at least one, or each, nucleic acid of the candidate mixture comprises a C-5 modified pyrimidine;
(b) exposing the candidate mixture to a slow off-rate enrichment process, wherein nucleic acids having a slow rate of dissociation from the target molecule relative to other nucleic acids in the candidate mixture bind the RIG-I protein, forming nucleic acid-target molecule complexes;
(c) partitioning slow off-rate nucleic acids from the candidate mixture;
(d) amplifying the slow off-rate nucleic acids to yield a mixture of nucleic acids enriched in nucleic acid sequences that are capable of binding to the RIG-I protein with a slow off-rate, whereby a slow off-rate aptamer to the RIG-I protein molecule is selected.

19. The method of claim 18, wherein the candidate mixture comprises nucleic acids comprising the sequence 5′- PEPSZV -3′ (SEQ ID NO: 49), wherein each P is independently, and for each occurrence, a C-5 modified pyrimidine; E is a C-5 modified pyrimidine, A, or G; S is a G or C; Z is a C-5 modified pyrimidine or A; and V is a C, A, or G.

20. The method of claim 18 or 19, wherein the candidate mixture comprises nucleic acids comprising the sequence 5′-PEPSFP-3′ (SEQ ID NO: 50), wherein each P is independently, and for each occurrence, a C-5 modified pyrimidine; E is a C-5 modified pyrimidine, A or G; S is a G or C; and F is a C-5 modified pyrimidine, C, G, or A.

21. The method of any one of claims 18-20, wherein the candidate mixture comprises nucleic acids comprising the sequence 5′-AAPGAPGAGG-3′ (SEQ ID NO: 51).

22. The method of any one of claims 18-21, wherein each nucleic acid is, independently, from 35 to 60 nucleotides in length, or from 35 to 50 nucleotides in length, or from 40 to 50 nucleotides in length.

23. The method of any one of claims 18-22, wherein each C-5 modified pyrimidine containing nucleoside is independently selected from: 5-(N-benzylcarboxyamide)-2′-deoxyuridine (BndU), 5-(N-benzylcarboxyamide)-2′-O-methyluridine, 5-(N-benzylcarboxyamide)-2′-fluorouridine, 5-(N-phenethylcarboxyamide)-2′-deoxyuridine (PEdU), 5-(N-thiophenylmethylcarboxyamide)-2′-deoxyuridine (ThdU), 5-(N-isobutylcarboxyamide)-2′-deoxyuridine (iBudU), 5-(N-tyrosylcarboxyamide)-2′-deoxyuridine (TyrdU), 5-(N-3,4-methylenedioxybenzylcarboxyamide)-2′-deoxyuridine (MBndU), 5-(N-4-fluorobenzylcarboxyamide)-2′-deoxyuridine (FBndU), 5-(N-3-phenylpropylcarboxyamide)-2′-deoxyuridine (PPdU), 5-(N-imidizolylethylcarboxyamide)-2′-deoxyuridine (ImdU), 5-(N-isobutylcarboxyamide)-2′-O-methyluridine, 5-(N-isobutylcarboxyamide)-2′-fluorouridine, 5-(N-tryptaminocarboxyamide)-2′-deoxyuridine (TrpdU), 5-(N-R-threoninylcarboxyamide)-2′-deoxyuridine (ThrdU), 5-(N-tryptaminocarboxyamide)-2′-O-methyluridine, 5-(N-tryptaminocarboxyamide)-2′-fluorouridine, 5-(N-[1-(3-trimethylamonium) propyl] carboxyamide)-2′-deoxyuridine chloride, 5-(N-naphthylmethylcarboxyamide)-2′-deoxyuridine (NapdU), 5-(N-naphthylmethylcarboxyamide)-2′-O-methyluridine, 5-(N-naphthylmethylcarboxyamide)-2′-fluorouridine, 5-(N-[1-(2,3-dihydroxypropyl)]carboxyamide)-2′-deoxyuridine), 5-(N-2-naphthylmethylcarboxyamide)-2′-deoxyuridine (2NapdU), 5-(N-2-naphthylmethylcarboxyamide)-2′-O-methyluridine, 5-(N-2-naphthylmethylcarboxyamide)-2′-fluorouridine, 5-(N-1-naphthylethylcarboxyamide)-2′-deoxyuridine (NEdU), 5-(N-1-naphthylethylcarboxyamide)-2′-O-methyluridine, 5-(N-1-naphthylethylcarboxyamide)-2′-fluorouridine, 5-(N-2-naphthylethylcarboxyamide)-2′-deoxyuridine (2NEdU), 5-(N-2-naphthylethylcarboxyamide)-2′-O-methyluridine, 5-(N-2-naphthylethylcarboxyamide)-2′-fluorouridine, 5-(N-3-benzofuranylethylcarboxyamide)-2′-deoxyuridine (BFdU), 5-(N-3-benzofuranylethylcarboxyamide)-2′-O-methyluridine, 5-(N-3-benzofuranylethylcarboxyamide)-2′-fluorouridine, 5-(N-3-benzothiophenylethylcarboxyamide)-2′-deoxyuridine (BTdU), 5-(N-3-benzothiophenylethylcarboxyamide)-2′-O-methyluridine, and 5-(N-3-benzothiophenylethylcarboxyamide)-2′-fluorouridine.

24. The method of any one of claims 18-22, wherein each C-5 modified pyrimidine containing nucleoside is 5-(N-3-phenylpropylcarboxyamide)-2′-deoxyuridine (PPdU).

25. The method of any one of claims 18-24, wherein the RIG-I protein is a human RIG-I protein.
